(19) United States
(12) Patent Application Publication
(10) Pub. No.: US 2022/0168342 A1
(43) Pub. Date: Jun. 2, 2022

(54) GENOME EDITED PRIMARY B CELL AND METHODS OF MAKING AND USING

(71) Applicant: REGENTS OF THE UNIVERSITY OF MINNESOTA, MINNEAPOLIS, MN (US)

(72) Inventors: Branden S. MORIARITY, Minneapolis, MN (US); John HUNZEKER, APO, AE (US); Matthew JOHNSON, Minneapolis, MN (US); Kanut LAOHARAWEE, Minneapolis, MN (US)

(73) Assignee: REGENTS OF THE UNIVERSITY OF MINNESOTA, MINNEAPOLIS, MN (US)

(21) Appl. No.: 16/332,555

(22) PCT Filed: Sep. 12, 2017
(86) PCT No.: PCT/US17/51182

§ 371 Date: Mar. 12, 2019

Related U.S. Application Data
(60) Provisional application No. 62/393,512, filed on Sep. 12, 2016.

(51) Int. Cl. A61K 35/17; C12N 5/0781; C12N 15/90; C12N 15/86; C12N 9/22; C12N 15/11

(52) U.S. Cl. CPC A61K 35/17; C12N 5/0635; C12N 15/907; C12N 15/86; C12N 9/22; C12N 2510/00; C12N 2740/15043; C12N 2501/2304; C12N 2501/52; C12N 2310/20; C12N 2800/80; C12N 15/11

(57) ABSTRACT

Genonie-edited primary B cells, methods of making genome-edited primary B cells, a therapeutic cassette that can be introduced into primary B cells, and methods of using the genome-edited primary B cells and the therapeutic cassette.

### CONTINUING APPLICATION DATA

This application claims the benefit of U.S. Provisional Application Ser. No. 62/393,512, filed Sep. 12, 2016, which is incorporated by reference herein.

### SEQUENCE LISTING

This application contains a Sequence Listing electronically submitted to the United States Patent and Trademark Office via EFS-Web as an ASCII text file entitled “110-05460201_ST25.txt” having a size of 90.3 kilobytes and created on Sep. 12, 2017. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

B lymphocytes are a component of the adaptive immune system described as a population of cells that express clonally diverse cell surface immunoglobulin (Ig) receptors recognizing specific antigenic epitopes. The process of B cell maturation appears to be largely conserved between humans and mice. Dysregulation of normal B cell development can lead to congenital immunodeficiencies, autoimmune diseases, and even leukemia or lymphoma. B cells can generate protective antibodies that last for decades after initial antigen exposure. After immunization, antigen-reactive Ig are detectable for many years due to the generation of long-lived plasma cells. These long-lived plasma cells can arise from long-lived antibody producing cells that do not proliferate or cells that arise from germinal centers during stages of antibody maturation. These plasma cells are believed to live for many years and potentially even decades.

### SUMMARY OF THE INVENTION

This disclosure describes genome-edited primary B cells, methods of making genome-edited primary B cells, a therapeutic cassette that can be introduced into primary B cells, and methods of using the genome-edited primary B cells and the therapeutic cassette.

In one aspect, this disclosure describes a genome-edited primary B cell. In some embodiments, the B cell includes a cell expressing at least one of CD19, IgM, IgD, CD27, CD21, and CXCR5.

In some embodiments, the B cell includes a cell isolated from peripheral blood, umbilical cord cells, ascites, or a solid tumor. In some embodiments, the B cell includes a non-clonal cell, a proliferating cell, a mammalian cell, and/or a human cell.

In some embodiments of the genome-edited primary B cell, an endogenous gene is deleted, a gene includes a point mutation, and/or the cell includes an exogenous gene. The gene can include a nucleic acid encoding at least a portion of a B cell receptor (BCR).

In some embodiments, the B cell exhibits decreased expression of an endogenous B cell receptor (BCR) relative to a non-genome edited primary B cell.

In some embodiments, the B cell includes a modification that alters expression or activity of CD19. In some embodiments, the B cell includes a modification of a noncoding region of the genome.

In some embodiments, the genome-edited primary B cell exhibits increased survival relative to a non-genome edited primary B cell.

In another aspect, this disclosure describes a method that includes administering to the subject a composition comprising a genome-edited primary B cell. In some embodiments, the method includes treating or preventing a disease in a subject; the disease can include, for example, an enzymopathy, a cancer, a precancerous condition, an infection with a pathogen, or a viral infection.

In another aspect, this disclosure describes a therapeutic cassette that includes a nucleic acid encoding a B cell receptor (BCR) and a nucleic acid encoding a gene to be overexpressed. The B cell receptor can include a transmembrane region. The gene to be overexpressed can include a nucleic acid encoding an enzyme. In some embodiments, the enzyme includes an enzyme lacking in a subject having an enzymopathy or having been diagnosed with an enzymopathy. In some embodiments, the nucleic acid encoding the BCR and the nucleic acid encoding the gene to be overexpressed are transcriptionally linked, translationally linked, or both.

In some embodiments, the therapeutic cassette includes a promoter that drives transcription of the nucleic acid encoding the BCR and the nucleic acid encoding the gene to be overexpressed.

In another aspect, this disclosure describes a cell that includes the therapeutic cassette. In some embodiments, the cell includes a B cell, and/or a long-lived plasma cell. The cell can include a modification of a nucleic acid encoding the endogenous B cell receptor (BCR).

In a further aspect, this disclosure describes a method that includes administering a cell that includes the therapeutic cassette. In some embodiments, the method can also include administering an antigen to the subject, wherein the BCR of the therapeutic cassette is specific to the antigen.

In yet another aspect, this disclosure describes a method including editing a genome of a primary B cell. The primary B cell can include a cell expressing CD19; a cell expressing IgM or IgD, or a combination thereof; a CD27⁺ cell; a CD21⁺ cell; and/or a CXCR5⁺ cell. In some embodiments, the primary B cell can include a cell isolated from peripheral blood, umbilical cord cells, ascites, or a solid tumor; a non-clonal cell; a proliferating cell; a mammalian cell; and/or a human cell.

In some embodiments, the method includes introducing an exogenous protein or nucleic acid into the primary B cell. The method can include electroporation of the cell. In some embodiments, the method includes introducing a targeted nuclease or a nucleic acid encoding a targeted nuclease (including, for example, Cas9 or a nucleic acid encoding Cas9). In some embodiments, the method includes introducing a guide RNA (gRNA). The gRNA can include a chemically modified gRNA. A chemically modified gRNA can include 2′-O-methyl (M), 2′-O-methyl-3′-phosphorothioate (MS), or 2′-O-methyl-3′-thiophosphonoacetate (MSP).

In some embodiments, the method includes introducing Natronobacterium gregoryi Argonaute (NgAgo) and a guide DNA (gDNA).

In some embodiments, editing the genome includes editing a gene including, for example, a nucleic acid encoding for CD19; editing a nucleic acid encoding a portion of a B cell receptor (BCR); and/or editing a noncoding region of the genome.

In some embodiments, the method further includes selecting a B cell. In some embodiments, selection is performed after editing the genome. In some embodiments, the B cell is selected for an edited genome.

In some embodiments, the method includes subjecting the primary B cell to at least one of an activation, a stimulation, and a proliferation step.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows delivery of mRNA encoding eGFP into stimulated primary human B cells. (A) Histograms depicting the percent eGFP positive B cells after gating on live cells. (B) Flow plots quantifying viability of B cells depicted in panel A based on APC e-Fluor 780 Fixable Viability Dye.

FIG. 2 shows exemplary results of a surveyor nuclease assay from stimulated primary human B cells treated with chemically modified gRNAs and Cas9 mRNA or protein. DNA was extracted 3 days after transfection. Lanes without gene modification rates are not labeled for simplicity but represent 0% editing. Lanes are numbered 1-5 (left to right) with the key denoting conditions used. Target genes are noted in brackets below each image.

FIG. 3 shows a schematic of some exemplary embodiments of targeted gene integration at the BCR heavy chain locus. (A) Diagram of the BCR heavy chain locus depicting the enhancers, D, J, and constant exons. A proposed site of transgene integration is indicated. (B) Diagram of two embodiments for cargo design for gene delivery at the BCR heavy chain locus. P2A: ribosomal skip sequence to link cDNAs transcriptionally, pA: polyadenylation sequence, Splice acc: strong splice acceptor element.

FIG. 4 shows exemplary results of lentiviral transduction of primary human B cells. Contour plots depict the percent of eGFP-express sing B cells after gating on live cells based on APC e-Fluor780 Fixable Viability Dye. BaEV-psuedotyped (Fusil et al., Molecular Therapy, 2015, 23(11):1734-47) (left column), VSVg-psuedotyped (middle column), and no lentivirus controls (right column) were tested in acutely stimulated (top row), chronically stimulated (center row), and unstimulated (bottom row) B cell cultures, as described in Example 2. B cells were more efficiently transduced across all conditions using the BaEV psuedotype virus compared to cells transduced with the standard VSVg psuedotype.

FIG. 5 shows exemplary cell surface expression of CD19 protein following knockout of the CD19 gene in primary human B cell cultures. The CD19 locus was targeted by electroporating Cas9 mRNA and chemically modified gRNA (TriLink) into stimulated B cells using the NEON Transfection System (1400 volts, 10 milliseconds (ms), 3 pulses). Cas9 mRNA without gRNA, and no-electroporation samples were included to demonstrate control levels of CD19. CD19 expression was measured by flow cytometry five days after electroporation. (A) Histograms depicting CD19 expression in Cas9+rRNA treated cells (solid line) vs. Cas9 alone treated cells (dashed line) and no-electroporation controls (solid grey background). (B) CD19 cell surface expression decreased from 96-98% in cells treated with Cas9 alone and in no electroporation controls to 38% in B cells treated with Cas9 and CD19 gRNA.

FIG. 6 shows exemplary vector constructs of plasmids encoding therapeutic cassettes used to engineer primary human B cells to express antibodies. These plasmids allow for expression of a membrane-bound B cell receptor (BCR) or a secreted antibody, depending on the maturation state of the B cells. (A) Schematic of a lentiviral vector constructed to express anti-PE heavy chain and light chain and codon-optimized alpha-L-iduronidase (coIDUA) under regulation of MND promoter. Co-expression of the heavy chain, the light chain and the coIDUA was obtained by introducing the P2A peptide sequences. Sequence is provided in Table 3. (B) Schematic of FAM1 and FAM2 lentiviral vectors (originally described in Fusil et al., Molecular Therapy, 2015, 23(11):1734-47) modified to express an anti-PE B cell receptor (BCR)/antibody. Sequences are provided in Tables 6 and 8. (C) Schematic of a lentiviral vector constructed to express B12 heavy chain and light chain and codon-optimized IDUA (coIDUA) under regulation of MND promoter. Co-expression of the heavy chain, the light chain and the coIDUA was obtained by introducing the P2A peptide sequences. Sequence is provided in Table 4. (D) Schematic of FAM1 and FAM2 lentiviral vectors (originally described in Fusil et al., Molecular Therapy, 2015, 23(11):1734-47) modified to express a B12 B cell receptor (BCR)/antibody. Sequences are provided in Tables 5 and 7.

FIG. 7 shows the impact of exemplary electroporation settings on the efficacy of transfecting DNA or RNA encoding eGFP into primary human B cells using the NEON Transfection System. (A) List of the conditions tested—voltage (volts); widths (milliseconds (ms)), and number of pulses are shown. (B) The impact of the electroporation conditions on transfection efficacy (top row), cell viability (middle row), and total cell counts (bottom row) were tested using either a GFP-encoding plasmid (left column) or GFP-encoding mRNA (right column).

FIG. 8 shows exemplary results of indel formation at the BCL2 locus using the Alt-R CRISPR-Cas9 system, as described in Example 2. Primary human B cells were electroporated using Neon Transfection System (1400 Volts, 10 ms, 3 pulses) with Alt-R CRISPR-Cas9 system (Integrated DNA Technology, Coralville, Iowa) targeting the BCL-2 locus. Sequencing analysis using TIDE program (available on the world wide web at tide.nki.nl) showed 11.3% total editing efficiency (R²=0.89). The majority of editing was observed to include either insertion of one nucleotide or deletion of 9 nucleotides.

FIG. 9 shows the impact of cell density on the growth and expansion of mature naïve-like B cells from CD19⁺ cells isolated from peripheral blood mononuclear cells following activation with CD40L crosslinking antibody (Miltenyi Biotech, Inc., San Diego, Calif.) and IL-4. Cells plated at a high-density concentration (1×10⁶ cells/mL) in HSC Expansion Media (Miltenyi Biotech, Inc., San Diego, Calif.) showed minimal expansion by day 14 compared to the 30-fold increase observed in cells plated at a low-density concentration (2×10⁵ cells/mL).

FIG. 10 shows exemplary (A) intracellular and (B) secreted IDUA activity of HEK 293T cell 3-day post-electroporation, as further described in Example 2.

### DETAILED DESCRIPTION

Because B cells can become long lived and inherently have the ability to generate large quantities of protein (i.e. antibody), B cells could provide an ideal platform for gene therapy including, for example, for treatment of enzymopathies. B cells are also readily available in peripheral blood (making up 1-7% of all leucocytes), and methods to expand the cells are readily available. Moreover, data suggest that cells cross the blood brain barrier more readily than proteins, potentially making cellular therapies for enzymopathies with brain involvement more desirable then enzyme replacement therapy. Yet the delivery of therapeutic genes to B cells using genome-engineering approaches or the use of any targeted nuclease in primary human B cells has not previously been reported.

This disclosure describes a genome-edited primary B cell; methods of making the genome-edited primary B cell; and methods of using the genome-edited primary B cell including, for example, administering the cell. This disclosure further describes a therapeutic cassette that can be introduced into a primary B cell, methods of making the therapeutic cassette, methods of making a B cell including the therapeutic cassette, and methods of using the therapeutic cassette.

### B Cell

In some embodiments, the B cell can be a CD19⁺ cell. In some embodiments, the B cell can be a primary B cell. As used herein, a “primary B cell” is a non-immortalized B cell. In some embodiments, a “primary B cell” is a B cell that is freshly isolated. In some embodiments, the B cell can be isolated from peripheral blood mononuclear cells (PBMCs). In some embodiments, the B cell can be derived from an iPSC. In some embodiments, the B cell can be derived from a population of CD34⁺ cells.

In some embodiments, a “primary B cell” is a B cell that has undergone up to 5 replications or divisions after being isolated, up to 10 replications or divisions after being isolated, up to 15 replications or divisions after being isolated, up to 20 replications or divisions after being isolated, up to 25 replications or divisions after being isolated, up to 30 replications or divisions after being isolated, up to 35 replications or divisions after being isolated, or up to 40 replications or divisions after being isolated.

In some embodiments, a “primary B cell” is a B cell that has undergone up to 5 replications or divisions after being derived, up to 10 replications or divisions after being derived, up to 15 replications or divisions after being derived, up to 20 replications or divisions after being derived, up to 25 replications or divisions after being derived, up to 30 replications or divisions after being derived, up to 35 replications or divisions after being derived, or up to 40 replications or divisions after being derived.

In some embodiments, the primary B cell is a non-clonal cell. In some embodiments, primary B cell is a proliferating cell. In some embodiment the B cell is preferably cultured in the presence of CD40L.

In some embodiments, the B cell can be a naïve B cell. In some embodiments, a “naïve B cell” is CD19⁺, IgD⁺, IgM⁺, CD27⁻, CD21⁺, and/or CXCR5⁺. In some embodiments, the B cell can be a memory B cell. In some embodiments, a “memory B cell” is CD19⁺, IgD⁻, CD27⁺, CD21⁺, and/or CXCR5⁺. In some embodiments, the B cell can be an activated memory B cell. In some embodiments, an “activated memory B cell” is CD19⁺, IgD⁻, CD27⁺, CD21⁻, and/or CXCR5⁺. In some embodiments, the B cell can be a natural effector B cell. In some embodiments, a “natural effector B cell” is CD19⁺, IgD⁺, IgM⁺, and/or CD27±. In some embodiments, the B cell can be a plasmablast. In some embodiments, a “plasmablast” is CD19⁺, CXCR5⁻, CD38⁺, CD27⁻, and/or CD20⁻.

In some embodiments, the B cell may be a B cell that has undergone class-switch recombination. In some embodiments, the B cell may be a B cell that has not undergone class-switch recombination.

In some embodiments, the B cell is a mammalian cell. In some embodiments, the B cell is preferably a human cell. In some embodiments, the B cell is a mouse cell.

### Genome Edited Primary B Cell

A primary B cell is “genome edited” if the primary B cell includes a modification to its genome compared to a non-genome edited B cell. In some embodiments, a non-genome edited B cell is a wild-type B cell. In some embodiments, a non-genome edited B cell is a freshly isolated B cell.

In some embodiments, the genome edited primary B cell includes a modification of a noncoding region of the genome and/or a coding region of the genome (e.g., a gene). In some embodiments, the noncoding region of the genome can include a sequence for a small, regulatory noncoding RNA, including, for example, a microRNA (miRNA). In some embodiments, the noncoding region of the genome is preferably involved in regulating the function, activation, and/or survival of the B cell.

In some embodiments, a portion of genomic information and/or a gene can be deleted. In some embodiments, a portion of genomic information and/or a gene can be added. In some embodiments, the genomic information and/or the gene that is added is exogenous. In some embodiments, “exogenous” genomic information or an “exogenous” gene can be genomic information or a gene from a non-B cell. In some embodiments, “exogenous” genomic information or an “exogenous” gene can be an additional copy of genomic information or a gene already present in the B cell. In some embodiments, “exogenous” genomic information or an “exogenous” gene can be genomic information or a gene from a cell of another species than the B cell being modified. In some embodiments, “exogenous” genomic information or an “exogenous” gene can be artificially generated including, for example, a nucleic acid encoding a chimeric antigen receptor. In some embodiments, a portion of genomic information and/or a gene can be altered, for example, by a point mutation.

In some embodiments, a genome edited primary B cell preferably includes a modification that alters expression or activity of the genome edited primary B cell relative to a non-genome edited primary B cell. For example, in some embodiments, the genome edited primary B cell may include a therapeutic cassette, as further described below.

In some embodiments, a genome edited primary B cell preferably includes a modification of a nucleic acid encoding the endogenous B cell receptor (BCR). In some embodiments, the modification results in a modification of the expression of the endogenous BCR. For example, expression of the endogenous BCR may be abrogated relative to a non-genome edited primary B cell. In some embodiments, the expression of the endogenous BCR may be enhanced relative to a non-genome edited primary B cell. In some embodiments, a genome edited primary B cell includes a modification of a nucleic acid encoding CD19. In some embodiments, a genome edited primary B cell includes a modification of a nucleic acid encoding a light chain.

In some embodiments, a genome edited primary B cell includes a modification of a nucleic acid encoding a cytokine. The cytokine can include, for example, IL-10, IL-4, IL-7, IL-2, IL-15, IL-6, or IFN-γ, or combinations thereof.

In some embodiments, a genome edited primary B cell includes a modification of a nucleic acid encoding a member of the Bcl-2 family including, for example BAX, also known as bcl-2-like protein 4, and bcl-2. In some embodiments, the modification of a nucleic acid encoding a member of the Bcl-2 family allows for increased survival of the genome edited primary B cell including, for example, increased survival in culture.

In some embodiments, a genome edited primary B cell includes a modification of a nucleic acid encoding a B cell inhibitory receptor including, for example, FCγRII, CD22, PD-1, CDS, CD66a, LAIR1, ILT2, or CD72, or combinations thereof. In some embodiments, the modification alters expression or activity of the inhibitory receptor relative to a non-genome edited primary NK cell. For example, expression of the inhibitory receptor can be decreased.

In some embodiments, a genome edited primary B cell includes a modification that affects the frequency or rate with which a B cell undergoes affinity maturation. In some embodiments, a genome edited primary B cell includes a modification of a nucleic acid encoding BCL6 and/or BLIMP1. BCL6 inhibits BLIMP1 expression, and BLIMP1 expression inhibits BCL6 expression. BCL6 expression can cause a B cell to be retained in the germinal center where it continues to undergo affinity maturation. BLIMP1 drives B cells to differentiate into plasma cells (both short lived and long lived). In some embodiments, the modification of a nucleic acid encoding BCL6 and/or BLIMP1 could be used to affect the frequency or rate with which a B cell undergoes affinity maturation.

In some embodiments, a genome edited primary B cell includes a modification in a nucleic acid encoding a member of an endoplasmic-reticulum (ER) stress response pathway including, for example, IRE1, PERK, or ATF6, or combinations thereof, under a temporary block of caspase-dependent cell death during their initial differentiation. In some embodiments, the modification in a nucleic acid encoding a member of an ER stress response pathway may affect the survival of the genome edited primary B cell and may, for example, increase survival in culture.

In some embodiments, the genome-edited primary B cell preferably includes a modification that alters survival of the genome edited primary B cell relative to a non-genome edited primary B cell. In some embodiments, the gene-edited primary B cell exhibits increased capacity to expand relative to a non-genome edited primary B cell. The expansion can be, for example, in vivo or in vitro. In some embodiments, the expansion can be in vitro after co-culturing with a cytokine, an antibody, an antigen, a cell expressing an antigen, or a combination thereof.

### Genome Editing of the B Cell

This disclosure also describes a method of making a genome-edited B cell including a genome-edited primary B cell.

In some embodiments, the method includes a technique to introduce a protein or nucleic acid into the primary B cell. Any suitable method of introducing a protein or nucleic acid may be used. In some embodiments, the method preferably includes electroporation of a primary B cell to introduce genetic material including, for example, DNA, RNA, and/or mRNA. As used herein, electroporation may include nucleofection. In some embodiments, the genetic material may be introduced via transduction with a virus including, for example adeno-associated virus (AAV), an integrase-deficient lentivirus (IDLV), etc. An adeno-associated virus can include any suitable serotype including, for example, AAV2, AAV3, AAV4, AAV5, AAV6), etc. Because plasmid DNA can be toxic to B cells, in some embodiments, mRNA or protein based approaches of genome editing are preferred. In some embodiments, a technique to introduce a protein or nucleic acid can include introducing a protein or nucleic acid via electroporation; microinjection; viral delivery; exosomes; liposomes; biolistics; jet injection; hydrodynamic injection; ultrasound; magnetic field-mediated gene transfer; electric pulse-mediated gene transfer; use of nanoparticles including, for example, lipid-based nanoparticles; incubation with a endosomolytic agent; use of cell-penetrating peptides; etc. In some embodiments, the method preferably includes electroporation of a primary B cell using a NEON transfection system.

In some embodiments, the method includes editing a gene. Editing a gene can include introducing one or more copies of the gene, altering the gene, deleting the gene, upregulating expression of the gene, downregulating expression of the gene, mutating the gene, methylating the gene, demethylating the gene, acetylating the gene, and/or deacetylating the gene. Mutating the gene can include introducing activing mutations, introducing inactivating and/or inhibitory mutations, and/or introducing point mutations.

In some embodiments, the method preferably includes inducing double stranded breaks in the genome of the primary B cell. Double stranded breaks may be introduced using a targeted nuclease including, for example, a transcription activator-like effector nucleases (TALEN), a zinc finger nuclease (ZFN), a CRISPR-associated nuclease, etc. In some embodiments, double stranded breaks are preferably introduced using the CRISPR/Cas9 system. In some embodiments, the method preferably includes introducing a CRISPR nuclease (including, for example, Cas9 and/or Cpf1) or DNA or RNA encoding a CRISPR nuclease (including, for example, DNA or RNA encoding Cas9 or Cpf1). The method can, in some embodiments, include introducing a guide RNA (gRNA).

In some embodiments, the method includes introducing a DNA-guided DNAse. In some embodiments, the method includes introducing Natronobacterium gregoryi Argonaute (NgAgo). In some embodiments, NgAgo can be used as a DNA-guided endonuclease. (Gao et al., Nature Biotechnology, 2016, doi:10.1038/nbt.354.) The method can further include, for example, introducing a guide DNA (gDNA).

The gRNA target or gDNA target can include any suitable target. In some embodiments, the target includes a portion of the B cell genome including, for example, a gene or a portion of a gene. In some embodiments, the targeted gene or portion of the gene enhances B cell function. For example, a gRNA target or gDNA target can include a B cell receptor, including, for example, a heavy chain gene, a light chain gene, or CD79; CD19; a B cell developmental regulator including, for example, BLIMP1 or BCL6; adeno-associated virus integration site 1 (AAVS1); a B cell inhibitory receptor (e.g., FCγRII, CD22, PD1, CDS, CD66a, LAIR1, ILT2, CD72, etc.); and/or a member of the ER stress response pathways (e.g., IREI, PERK, ATF6, etc.) In some embodiments, where transfection may be used to deliver the CRISPR/Cas9 system, the gRNA may preferably include a chemically modified gRNA. In some embodiments, the chemical modification to the gRNA preferably decreases a cell's ability to degrade the RNA. In some embodiments, a chemically modified gRNA includes one or more of the following modifications: 2′-fluoro (2′-F), 2′-O-methyl (2′-O-Me), S-constrained ethyl (cEt), 2′-O-methyl (M), 2′-O-methyl-3¹-phosphorothioate (MS), and/or 2′-O-methyl-3′-thiophosphonoacetate (MSP). In some embodiments, the chemically modified gRNA can include a gRNA and/or a chemical modification described in Hendel et al, Nature Biotechnology, 2015, 33(9):985-989 or Randar et al., PNAS, 2015, 112(51):E7110-7.

In such embodiments, the genome editing may occur via homologous recombination (HR) and/or non-homologous end joining (NHEJ) pathways including, for example, by microhomology-mediated end joining (MMEJ).

In some embodiments, the method includes selecting a B cell. In some embodiments, the selection is performed after editing a gene. A B cell can, in some embodiments, be selected using one or more of the following methods: flow sorting (including, for example, for GFP expression); magnetic bead separation (including, for example, targeting a cell-surface marker); transient drug resistance gene expression (including, for example, antibiotic resistance). In some embodiments, the selection may be for a B cell that has an edited genome.

In some embodiments, the method includes expanding an edited B cell. In some embodiments, the expansion can be performed after selecting the B cell. In some embodiments, a B cell can be expanded by co-incubation with an antigen recognized by the B cell receptor or a cell expressing an antigen recognized by the B cell receptor. In some embodiments, a B cell can be expanded by co-incubation with a cytokine or ligand including, for example, CD40L and/or IL-4.

### Methods of Transfection

In some embodiments, the primary B cells at the time of electroporation or transfection are preferably stimulated cells, that is, the cells have been subjected to an activation, a stimulation, and/or a proliferation step.

In some embodiments, the B cell can be stimulated for at least 12 hours, at least 18 hours, at least 1 day, at least 2 days, at least 3 days, at least 4 days, at least 5 days, at least 6 days, or at least 7 days. In some embodiments, the B cell can be simulated for up to 1 day, up to 2 days, up to 3 days, up to 4 days, up to 5 days, up to 6 days, up to 7 days, up to 8 days, up to 9 days, up to 10 days, up to 12 days, up to 14 days, up to 3 weeks, up to 4 weeks, or up to two months. In some embodiments, the B cell is preferably stimulated for 14 days.

In some embodiments, the B cell can be stimulated with cytokines. The cytokines can include, for example, IL-4, IL-7, IL-21, and/or B cell activating factor (BAFF). In some embodiments, the B cell can be stimulated by cross-linking a cell surface receptor, including, for example, CD40 (ligated and/or crosslinked, for example, by CD40L or anti-CD40 antibody).

In some embodiments, the B cell is preferably transfected via electroporation. The electroporation protocols may be optimized including, for example, by altering the number of B cells per reaction (e.g., 0.1 million, 0.5 million cells, 1 million cells, 2 million cells, 3 million cells, 4 million cells, or 5 million cells), the number of pulses (e.g., 1, 2, 3, 4, 5, 6, 7, or 8), the voltage (e.g., 1000 volts, 1100 volts, 1200 volts, 1300 volts, 1500 volts, or 1600 volts), the amount of nucleic acid (e.g., 0.5 μg, 1 μg, 2 μg, 5 μg, 10 μg, 15 μg, 20 μg, 25 μg, 30 μg, 35 μg, 40 μg, 45 μg, or 50 μg) and the duration of the pulse(s) (e.g., 2 ms, 5 ms, 7 ms, 9 ms, 10 ms, 11 ms, 13 ms, or 15 ms).

In some embodiments, the B cell can be electroporated using an AMAXA nucleofector or NEON system. In some embodiments, 1 million B cells per reaction may be electroporated using the NEON platform, 25 μg mRNA, and a protocol of 3 pulses, 1400 volts, and 10 ms duration.

In some embodiments, transfection levels are preferably tested 48 hours post-transfection. In some embodiments, transfection levels are preferably tested 72 hours post-transfection. In some embodiments, transfection levels are preferably tested using transfection of eGFP mRNA and by performing flow cytometry analysis at 48 hours and/or 72 hours post transfection to assess eGFP expression levels and cell viability.

In one embodiment, to test the efficiency and toxicity of mRNA delivery to primary human B cells, peripheral blood mononuclear cells were isolated from leukopaks using standard Ficoll-Paque separation. B cells were then isolated from PBMCs using the EasySep Human CD19 Positive Selection Kit (Stem Cell Technologies, Vancouver, Canada) and cultured in X-VIVO 20 media (Lonza Group, Ltd, Allendale N.J.) with 10% Human Serum. B cells were electroporated with in vitro transcribed mRNA encoding eGFP (TriLink BioTechnologies. San Diego, Calif.) using either the AMAXA or NEON electroporation platform. Preliminary results using this approach were disappointing with fewer than 6% EGFP⁺ B cells being detected at 48 hours post-electroporation, as measured by flow cytometry analysis.

In contrast, when the primary B cells were stimulated and expanded prior to electroporation, including, for example, using a seven-day culture with IL-4 and CD40L (Miltenyi Biotech, Inc., San Diego, Calif.), an increase in the percentage of eGFP positive cells 72 hours post-electroporation was observed, up to 97.6% EGFP⁺ B cells, along with a viability of up to 68.6%.

A major barrier to the application of CRISPR/Cas9 technology is the low rate of gene modification in some types of cells including hard-to-transfect cells, primary cells of various kinds, and other cells that cannot be cloned (i.e., propagated from single isolated cells). For example, initial attempts using unmodified gRNAs were unable to induce detectable double strand breaks in primary human T cells or CD34⁺ cells.

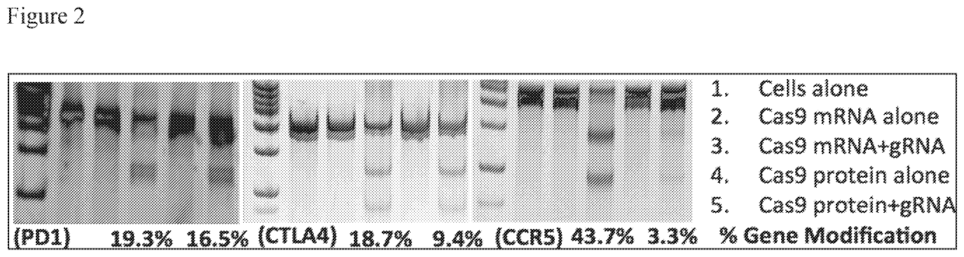

To determine if using the CRISPR/Cas9 system would allow for targeted gene delivery gene delivery to B cells either homologous recombination (HR) or non-homologous end joining (NHEJ) pathways, double strand break (DSB) induction was examined. Using gRNAs synthesized as RNA oligonucleotides containing 3 tandem 2-O-methyl-3-phosphorothioate modified bases on the 5′ and 3′ ends and Cas9 mRNA or protein, gene modification was induced (FIG. 2). These data demonstrate that targeted double strand breaks can be induced in B cells.

### Therapeutic Cassette

In some embodiments, the genome edited primary B cell may include a therapeutic cassette. In some embodiments, the therapeutic cassette preferably includes a nucleic acid encoding a BCR and a nucleic acid encoding a gene to be overexpressed. In some embodiments, the gene to be overexpressed preferably includes a nucleic acid encoding an enzyme. The nucleic acid encoding the BCR and the nucleic acid encoding the gene to be overexpressed are preferably transcriptionally and/or translationally linked.

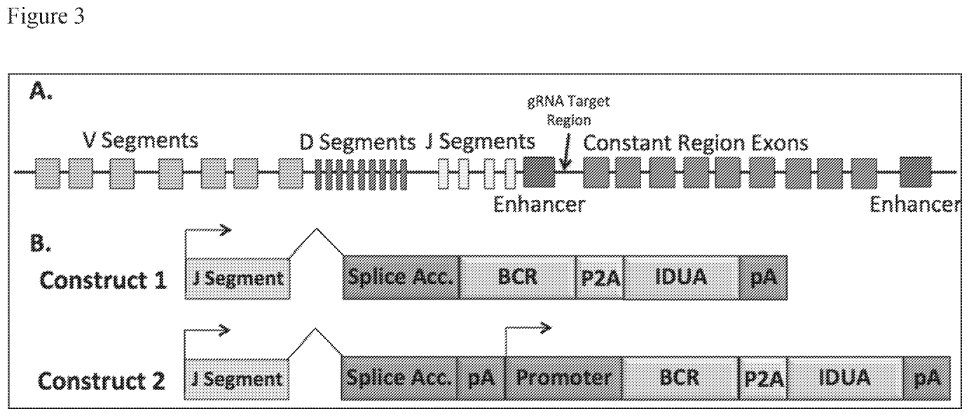

In some embodiments, it may be desired to inactivate the endogenous BCR as it could interfere with the functionality of a BCR transgene. In some embodiments, it may be preferable to insert a therapeutic cassette at the endogenous BCR heavy chain locus. In some embodiments, it may be preferable to edit the endogenous BCR by the transfection methods described herein. Because VDJ recombination removes some regions of the endogenous heavy chain locus, in some embodiments, genome editing, including, for example, insertion of a therapeutic cassette, can be targeted to near an enhancer found in the constant region. In some embodiments, the therapeutic cassette can be targeted to the region show in FIG. 3A, a region that is retained in nearly all heavy chain recombination events.

In some embodiments, the therapeutic cassette preferably includes a nucleic acid encoding a BCR. In some embodiments, the BCR is specific to an antigen that can be administered to a subject via immunization. In some embodiments, the BCR preferably includes a transmembrane region and/or a membrane bound-antibody. As used herein, a BCR may include either a membrane-anchored BCR or a soluble Ig or both.

In some embodiments, the therapeutic cassette includes a nucleic acid that encodes a heavy chain. In some embodiments, the transcription of the nucleic acid encoding a heavy chain can be driven by an endogenous promoter. In some embodiments, the transcription of the nucleic acid encoding a heavy chain can be driven by an exogenous promoter. In some embodiments, the promoter can include, for example, a MND promoter, a CMV promoter, a CAG promoter, a PGK promoter, a EF1A promoter, a FEEK promoter, etc. In some embodiments, the nucleic acid encoding the heavy chain preferably encodes a single variable segment, a single diversity segment, a single joining segment, and a single C-region. In some embodiments, the nucleic acid encoding the heavy chain preferably encodes a transmembrane region including, for example, an M1 and/or an M2 domain.

In some embodiments, the therapeutic cassette can include a nucleic acid encoding a light chain. In some embodiments, the transcription of the nucleic acid encoding a light chain can be driven by an endogenous promoter. In some embodiments, the transcription of the nucleic acid encoding a light chain can be driven by an exogenous promoter. In some embodiments, the promoter can include, for example, a MND promoter, a CMV promoter, a CAG promoter, a PGK promoter, a EF1A promoter, a FEEK promoter, etc. In some embodiments, the nucleic acid encoding the light chain preferably encodes a single variable segment, a single joining segment, and a single C-region.

In some embodiments, expression of the nucleic acid encoding the light chain can be transcriptionally or translationally linked to expression of the nucleic acid encoding the heavy chain including, for example, by internal ribosomal entry sites (IRESs), a 2A peptide sequence, a “2A-like” sequence, ribosomal skipping, and/or a CHYSEL (cis-acting hydrolase element) sequence. The 2A peptide sequence impairs normal peptide bond formation through a mechanism of ribosomal skipping, allowing the expression of more than one protein without introducing an internal ribosome entry sites (IRES) or an additional promoters. In some embodiments, 2A peptide can be derived from the porcine teschovirus-1 (P2A), the foot and mouth disease virus (F2A), or the Thosea asigna virus (T2A).

In some embodiments, the heavy chain and/or the light chain of the therapeutic cassette is specific to an antigen that can be administered to a subject via immunization. For example, in some embodiments, the heavy chain and/or the light chain can be specific for phycoerythrin (PE). In another example, in some embodiments, the heavy chain and/or the light chain can be specific for B12, an anti-HIV envelope protein.

The therapeutic cassette includes a nucleic acid encoding a gene to be overexpressed. In some embodiments, transcription of the nucleic acid encoding the gene to be overexpressed is preferably driven by the same promoter that drives transcription of at least one of the heavy chain or the light chain of the BCR. In some embodiments, transcription of the nucleic acid encoding the gene to be overexpressed can be driven by a different promoter than the promoter that drives transcription of at least one of the heavy chain or the light chain of the BCR.

When, for example, transcription of the nucleic acid encoding the gene to be overexpressed is driven by the same promoter that drives transcription of at least one of the heavy chain or the light chain of the BCR, overexpression of the gene can be controlled by immunizing a subject for the antigen recognized by the exogenous BCR. Moreover, in some embodiments, whether the therapeutic cassette produces a membrane anchored-form of the BCR or a soluble Ig-form of the BCR is dependent on the maturation state of the B cell into which the therapeutic cassette is targeted.

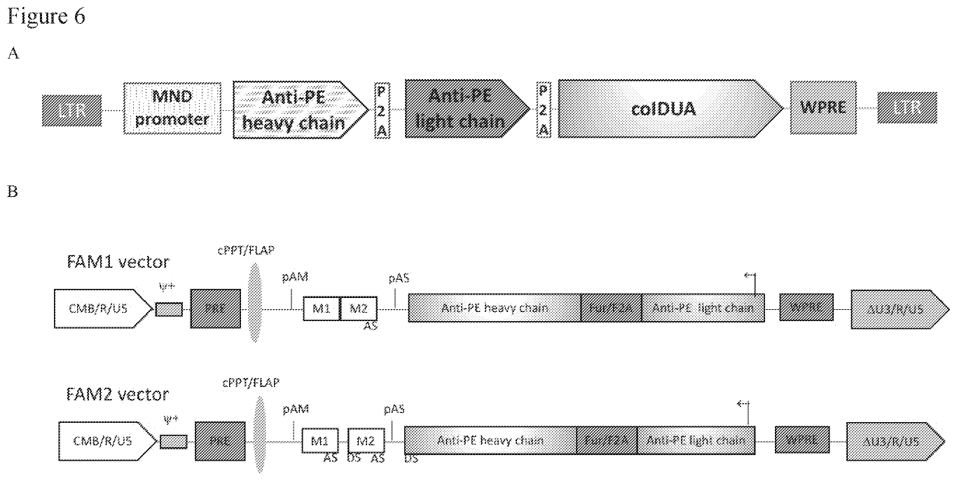

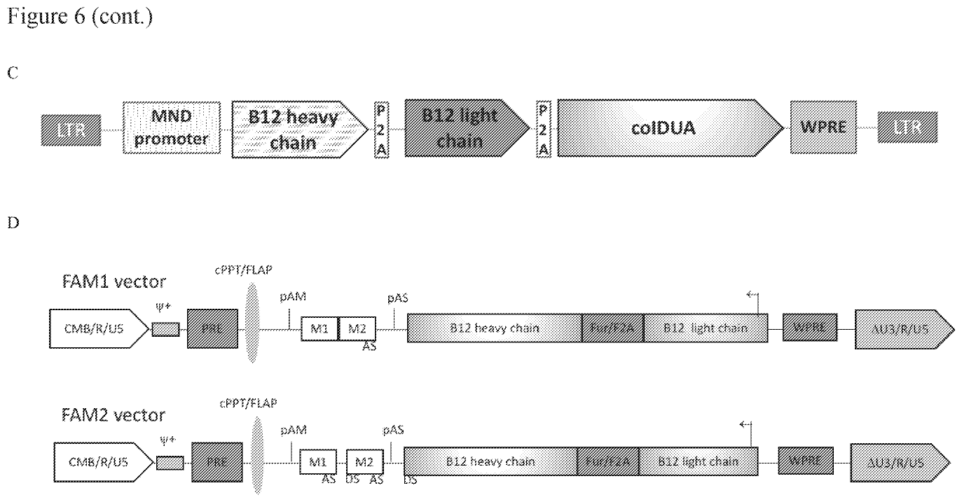

In some embodiments, the therapeutic cassette can include the components shown in FIG. 3B. In some embodiments, the therapeutic cassette can include the components arranged as shown in FIG. 3B. In some embodiments, the therapeutic cassette can include the components shown in at least one panel of FIG. 6. In some embodiments, the therapeutic cassette can include the components arranged as shown in at least one panel of FIG. 6.

In some embodiments, expression of the nucleic acid encoding the enzyme can be transcriptionally or translationally linked to BCR expression including, for example, by internal ribosomal entry sites (IRESs), a 2A peptide sequence, a “2A-like” sequence, ribosomal skipping, and/or a CHYSEL (cis-acting hydrolase element) sequence. In some embodiments, a splice acceptor approach or a constitutive promoter can be used to drive a nucleic acid encoding BCR linked to a nucleic acid encoding a therapeutic enzyme.

In some embodiments, the gene to be overexpressed preferably includes an enzyme and/or a therapeutic enzyme. A therapeutic enzyme can include, for example, an enzyme lacking in a subject having an enzymopathy. The enzymopathy can include, for example, Gaucher disease, Fabry disease, MPS I, MPS II (Hunter syndrome), MPS VI, Glycogen storage disease type II, Adenosine

Deaminase Deficiency, or Pompe disease. In some embodiments, the therapeutic enzyme includes alpha-L-iduronidase (IDUA), an enzyme essential for the breakdown of glycosaminoglycans (GAGs). A therapeutic enzyme can additionally or alternatively include, for example, an enzyme whose expression increases the health of a subject.

In some embodiments, the therapeutic cassette includes a nucleic acid encoding a marker gene including, for example, a gene for GFP or a gene for drug resistance.

In some embodiments, a nucleic acid encoding a heavy chain, a nucleic acid encoding a light chain, and a nucleic acid encoding a gene to be overexpressed are included in the therapeutic cassette and are transcriptionally linked. In some embodiments, the therapeutic cassette further includes one or more 2A peptides that are transcriptionally linked to the nucleic acid encoding a heavy chain, the nucleic acid encoding a light chain, and the therapeutic enzyme.

The therapeutic cassette may be encoded by a vector construct. In some embodiments, the vector construct includes a plasmid. In some embodiments, the vector may include a lentiviral vector including, for example, a BaEV-psuedotype lentiviral vector, a VSVg-psuedotype lentiviral vector, a FAM1 lentiviral vector, and/or FAM2 lentiviral vector (see Fusil et al., Molecular Therapy, 2015, 23(11):1734-47). In some embodiments, the vector may include a cis-acting DNA element including, for example, a gene encoding the posttranscriptional regulatory element of woodchuck hepatitis virus (WPRE), a gene encoding murine intracisternal type A particle, one or more copies of a gene encoding a constitutive transport element (CTE) originating from different simian retroviruses, etc.

Genome-Edited Plasma Cell and Methods of Differentiating a Genome-Edited Primary B Cell into a Genome-Edited Plasma Cell

This disclosure further provides a genome-edited plasma cell and methods for differentiating a genome-edited primary B cell into a long-lived plasma cell. In some embodiments, the genome-edited primary B cell and the genome-edited plasma cell preferably include a modification of a nucleic acid encoding the endogenous B cell receptor (BCR). In some embodiments, the expression of the endogenous BCR is abrogated relative to a non-genome edited primary B cell and the expression of an exogenous BCR is enhanced relative to a non-genome edited primary B cell.

In some embodiments, the BCR of the genome-edited primary B cell is specific to an antigen that can be administered to a subject via immunization. For example, in some embodiments, the B cell receptor can be specific for phycoerythrin (PE). In another example, in some embodiments, the B cell receptor can be specific for B12, an anti-HIV envelope protein.

In some embodiments, the subject including genome-edited primary B cell is exposed to an antigen recognized by the BCR. In some embodiments, administering an antigen to a subject via immunization where the BCR of the genome-edited primary B cell is specific to the antigen results in the generation of long lived-plasma cells. A BCR can be considered specific to an antigen when the antigen binding site of the BCR binds to the antigen.

Replacing the endogenous B cell receptor with a B cell receptor of known specificity allows for the transcriptional regulation of a nucleic acid under the same promoter as the components of the B cell receptor.

For example, a genome-edited primary B cell including a B cell receptor specific for phycoerythrin (PE) and including a nucleic acid encoding alpha-L-iduronidase (IDUA) could be introduced into a subject. The subject could be IDUA deficient. Immunizing the subject with PE could result in differentiation of the genome-edited primary B cell into a long-lived plasma cell and transcription of the nucleic acid encoding IDUA, thereby increasing the expression of IDUA in the genome-edited B cell and body-wide in the patient for cross correction of the disease. In other embodiments, the B cell specificity may be altered and/or the nucleic acid encoding a gene to be overexpressed can be modified.

### Administration

This disclosure further provides methods for using the genome-edited primary B cell described herein. For example, a genome-edited primary B cell can be used to treat or prevent a disease in a subject. A method can include administering to the subject a composition that includes the genome-edited primary B cell described herein or produced by a method described herein. The disease could include, for example, an enzymopathy, a cancer, a precancerous condition, infection with a pathogen (including, for example, malaria), or infection with a virus.

A genome-edited primary B cell can be administered to a subject alone or in combination with one or more other therapies. For example, a genome-edited primary B cell can be administered to a subject in combination a pharmaceutical composition that includes the active agent and a pharmaceutically acceptable carrier and/or in combination with a cellular therapy including, for example, a chimeric antigen receptor T cell (CAR-T). The B cell can be administered to a patient, preferably a mammal, and more preferably a human, in an amount effective to produce the desired effect. The B cell can be administered by a variety of routes, including, for example, intravenously, intratumorally, intraarterially, transdermally, via local delivery by catheter or stent, via a needle or other device for intratumoral injection, subcutaneously, etc. The B cell can be administered once or multiple times. A physician having ordinary skill in the art can determine and prescribe the effective amount and dosing of a genome-edited primary B cell and, optionally, the pharmaceutical composition required.

The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CML), Hodgkin's disease, and/or multiple myeloma. The cancer can be a metastatic cancer.

In a further aspect, a genome-edited primary B cell can be administered to inhibit the growth of a tumor in a subject. In some embodiments, the tumor can include a solid tumor.

The virus can include, for example, a herpes virus, including for example, CMV, Varicella zoster virus (VZV), Epstein-Barr virus (EBV), a herpes simplex virus (HSV) or Kaposi's sarcoma-associated herpesvirus (KSHV); a virus of the family Flaviviridae including for example, Dengue or Zika virus; or a lentivirus, including for example, human immunodeficiency virus (HIV).

The enzymopathy can include, in some embodiments, an enzymopathy that is currently treated with enzyme replacement therapy including, for example, Gaucher disease, Fabry disease, MPS I, MPS II (Hunter syndrome), MPS VI, Glycogen storage disease type II, Adenosine Deaminase Deficiency, or Pompe disease. The enzymopathy can include, in some embodiments, an enzymopathy that is currently treated with gene therapy.

A genome-edited primary B cell can be administered or prepared in a subject before, during, and/or after other treatments. Such combination therapy can involve administering a genome-edited primary B cell before, during, and/or after the use of other anti-cancer agent, other anti-viral agents, or a combination of other anti-cancer agent and other anti-viral agents. Such agents can include, for example, a cytokine; a chemokine; a therapeutic antibody including, for example, a high affinity anti-CMV IgG antibody; an NK cell receptor ligand, including, for example, BiKE or TRiKE; an adjuvant; an antioxidant; a chemotherapeutic agent; and/or radiation. The administration or preparation of the genome-edited primary B cell can be separated in time from the administration of other anti-cancer agents and/or other anti-viral agents by hours, days, or even weeks. Additionally or alternatively, the administration or preparation can be combined with other biologically active agents or modalities such as, but not limited to, an antineoplastic agent, and non-drug therapies, such as, but not limited to, surgery.

In some embodiments, a genome-edited primary B cell including a B cell receptor of known specificity can be administered to a subject before the subject is immunized with antigen recognized by the B cell receptor. In such embodiments, immunization with the antigen can allow for the transcriptional regulation of a nucleic acid under the same promoter as the components of the B cell receptor.

For example, a genome-edited primary B cell including a B cell receptor specific for an antigen and including a nucleic acid encoding an enzyme could be introduced into a subject. The subject could be enzyme deficient. Immunizing the subject with the antigen is expected to result in differentiation of the genome-edited primary B cell into a long-lived plasma cell and transcription of the nucleic acid encoding the enzyme, thereby increasing the expression of enzyme in the genome-edited B cell.

### Illustrative Embodiments of a Genome-Edited Primary B Cell

1. A genome-edited primary B cell.
2. The genome-edited primary B cell of embodiment 1, wherein the B cell comprises a cell expressing CD19.
3. The genome-edited primary B cell of either of embodiments 1 or 2, wherein the B cell comprises a cell expressing IgM or IgD, or a combination thereof.
4. The genome-edited primary B cell of any one of embodiments 1 to 3, wherein the B cell comprises a CD27⁺ cell.
5. The genome-edited primary B cell of any one of embodiments 1 to 4, wherein the B cell comprises a CD21⁺ cell.
6. The genome-edited primary B cell of any one of embodiments 1 to 5, wherein the B cell comprises a CXCR5⁺ cell.
7. The genome-edited primary B cell of any one of embodiments 1 to 6, wherein the B cell comprises a cell isolated from peripheral blood, umbilical cord cells, ascites, or a solid tumor.
8. The genome-edited primary B cell of any one of embodiments 1 to 7, wherein the B cell comprises a non-clonal cell.
9. The genome-edited primary B cell of any one of embodiments 1 to 8, wherein the B cell comprises a proliferating cell.
10. The genome-edited primary B cell of any one of embodiments 1 to 9, wherein the B cell is a mammalian cell.
11. The genome-edited primary B cell of any one of embodiments 1 to 10, wherein the B cell is a human cell.
12. The genome-edited primary B cell of any one of embodiments 1 to 11, wherein an endogenous gene of the genome-edited primary B cell is deleted.
13. The genome-edited primary B cell of any one of embodiments 1 to 12, wherein an endogenous of the genome-edited primary B cell comprises a point mutation.
14. The genome-edited primary B cell of any one of embodiments 1 to 13, the genome-edited primary B cell comprising an exogenous gene.
15. The genome-edited primary B cell of any one of embodiments 12 to 14, wherein the gene comprises a nucleic acid encoding at least a portion of a B cell receptor (BCR).
16. The genome-edited primary B cell of any one of embodiments 1 to 15 wherein the B cell exhibits decreased expression of an endogenous B cell receptor (BCR) relative to a non-genome edited primary B cell.
17. The genome-edited primary B cell of any one of embodiments 1 to 16, wherein the B cell comprises a modification that alters expression or activity of CD19.
18. The genome-edited primary B cell of any one of embodiments 1 to 17, wherein the B cell comprises a modification of a noncoding region of the genome.
19. The genome-edited primary B cell of any one of embodiments 1 to 18, wherein the genome-edited primary B cell exhibits increased survival relative to a non-genome edited primary B cell.
20. The genome-edited primary B cell of any one of embodiments 1 to 19, wherein the genome-edited primary B cell comprises a therapeutic cassette comprising a nucleic acid encoding a B cell receptor (BCR) and a nucleic acid encoding a gene to be overexpressed.
21. A method for treating or preventing a disease in a subject, the method comprising:

administering to the subject a composition comprising the genome-edited primary B cell of any one of embodiments 1 to 20.

22. The method of embodiment 21, wherein the disease comprises an enzymopathy, a cancer, a precancerous condition, an infection with a pathogen, or a viral infection.

### Illustrative Therapeutic Cassette Embodiments

1. A therapeutic cassette comprising a nucleic acid encoding a B cell receptor (BCR) and a nucleic acid encoding a gene to be overexpressed.
2. The therapeutic cassette of embodiment 1, wherein the BCR comprises a transmembrane region.
3. The therapeutic cassette of embodiment 1 or embodiment 2, wherein the gene to be overexpressed comprises a nucleic acid encoding an enzyme.
4. The therapeutic cassette of embodiment 3, wherein the enzyme comprises an enzyme lacking in a subject having an enzymopathy.
5. The therapeutic cassette of embodiment 4, wherein the enzyme comprises alpha-L-iduronidase (IDUA).
6. The therapeutic cassette of any one of embodiments 1 to 5, wherein the nucleic acid encoding the BCR and the nucleic acid encoding the gene to be overexpressed are transcriptionally linked, translationally linked, or both.
7. The therapeutic cassette of any one of embodiments 1 to 6, wherein the therapeutic cassette comprises a promoter that drives transcription of the nucleic acid encoding the BCR and the nucleic acid encoding the gene to be overexpressed.
8. The therapeutic cassette of any one of embodiments 1 to 7, wherein the BCR comprises a BCR specific for phycoerythrin (PE).
9. The therapeutic cassette of any one of embodiments 1 to 7, wherein the BCR comprises a BCR specific for B12.
10. A vector comprising the therapeutic cassette of any one of embodiments 1 to 9.
11. The vector of embodiment 10, wherein the vector comprises a lentiviral vector.
12. The vector of either of embodiment 10 or embodiment 11, wherein the vector comprises at least one of a BaEV-psuedotype lentiviral vector, a VSVg-psuedotype lentiviral vector, a FAM1 lentiviral vector, and a FAM2 lentiviral vector.
13. The vector of any one of embodiments 10 to 12, wherein the vector comprises a cis-acting DNA element.
14. A cell comprising the therapeutic cassette of any one of embodiments 1 to 9.
15. A cell comprising the vector of any one of embodiments 10 to 13.
16. The cell of either of embodiment 14 or embodiment 15, wherein the cell comprises a modification of a nucleic acid encoding an endogenous B cell receptor (BCR).
17. The cell of embodiment 16, wherein the modification comprises a modification that reduces expression of the endogenous BCR.
18. The cell of any one of embodiments 15 to 17, wherein the cell comprises a B cell.
19. The cell of any one of embodiments 15 to 18, wherein the cell comprises a long-lived plasma cell.
20. A method comprising administering the cell of any one of embodiments 14 to 19 to a subject.
21. The method of embodiment 20, the method further comprising administering an antigen to the subject, wherein the BCR of the therapeutic cassette is specific to the antigen.

### Illustrative Embodiments of Methods of Editing a Genome of a Primary B Cell

1. A method comprising editing a genome of a primary B cell.
2. The method of embodiment 1, wherein the primary B cell comprises a cell expressing CD19.
3. The method of either of embodiments 1 or 2, wherein the primary B cell comprises a cell expressing IgM or IgD, or a combination thereof.
4. The method of either of embodiments 1 or 3, wherein the primary B cell comprises a CD27⁺ cell.
5. The method of any one of embodiments 1 to 4, wherein the primary B cell comprises a CD21⁺ cell.
6. The method of any one of embodiments 1 to 5, wherein the primary B cell comprises a CXCR5⁺ cell.
7. The method of any one of embodiments 1 to 6, wherein the primary B cell comprises a cell isolated from peripheral blood, umbilical cord cells, ascites, or a solid tumor.
8. The method of any one of embodiments 1 to 7, wherein the primary B cell comprises a non-clonal cell.
9. The method of any one of embodiments 1 to 8, wherein the primary B cell comprises a proliferating cell.
10. The method of any one of embodiments 1 to 9, wherein the primary B cell is a mammalian cell.
11. The method of any one of embodiments 1 to 10, wherein the primary B cell is a human cell.
12. The method of any one of embodiments 1 to 11, the method comprising introducing an exogenous protein or nucleic acid into the primary B cell.
13. The method of any one of embodiments 1 to 12, the method comprising electroporation of the cell.
14. The method of any one of embodiments 1 to 13, the method comprising introducing a targeted nuclease or a nucleic acid encoding a targeted nuclease.
15. The method of any one of embodiments 1 to 14, the method comprising introducing a guide RNA (gRNA).
16. The method of embodiment 15, wherein the gRNA comprises a chemically modified gRNA.
17. The method of embodiment 16, wherein the chemically modified gRNA comprises 2′-O-methyl (M), 2′-O-methyl-3′-phosphorothioate (MS), or 2′-O-methyl-3′-thiophosphonoacetate (MSP).
18. The method of any one of embodiments 1 to 17, the method comprising introducing Natronobacterium gregoryi Argonaute (NgAgo) and a guide DNA (gDNA).
19. The method of any one of embodiments 1 to 18, wherein editing the genome comprises editing a gene for CD19.
20. The method of any one of embodiments 1 to 19, wherein editing the genome comprises editing a nucleic acid encoding a portion of a B cell receptor (BCR).
21. The method of any one of embodiments 1 to 20, wherein editing the genome comprises editing a noncoding region of the genome.
22. The method of any one of embodiments 1 to 21, wherein the method further comprises selecting a B cell.
23. The method of embodiment 22, wherein the selection is performed after editing the genome.
24. The method of either of embodiments 22 or 23, wherein the B cell is selected for an edited genome.
25. The method of any one of embodiments 1 to 24, wherein the method further comprises subjecting the primary B cell to at least one of an activation, a stimulation, and a proliferation step.
26. The method of embodiment 25, wherein subjecting the primary B cell to at least one of an activation, a stimulation, and a proliferation step comprises exposing the B cell to cytokines.
27. The method of either of embodiments 25 or 26, wherein subjecting the primary B cell to at least one of an activation, a stimulation, and a proliferation step comprises exposing the B cell to CD40L.
28. The method of any one of embodiments 25 to 27, wherein the primary B cell is subjected to at least one of an activation, a stimulation, and a proliferation step prior to introducing an exogenous protein or nucleic acid into the primary B cell.
29. The method of any one of embodiments 1 to 28, wherein the method further comprises introducing into the primary B cell a therapeutic cassette comprising a nucleic acid encoding a B cell receptor (BCR) and a nucleic acid encoding a gene to be overexpressed.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Medias

Culturing media:
X-VIVO 20 (Lonza Group, Ltd, Allendale, N.J.)
10% Human Serum

or
HSC Expansion Media XF (Miltenyi Biotec, San Diego, Calif.
5% Human Serum

Freezing Media:
45 mL fetal bovine serum (FBS) (heat inactivated)
5 mL DMSO

### Methods

### Design and Construction of Guide RNAs

Guide RNAs (gRNAs), shown in Table 1, were designed to the desired region of a gene using the CRISPR Design Program (Zhang Lab, MIT 2015). Multiple gRNAs were chosen based on the highest ranked values determined by off-target locations. The gRNAs were ordered in oligonucleotide pairs: 5′-CACCG-gRNA sequence-3′ and 5′-AAAC-reverse complement gRNA sequence-C-3′. The gRNAs were cloned together using a modified version of the target sequence cloning protocol (Zhang Lab, MIT). The oligonucleotide pairs were phosphorylated and annealed together using T4 PNK (NEB) and 10×T4 Ligation Buffer (NEB) in a thermocycler with the following protocol: 37° C. 30 minutes, 95° C. 5 minutes and then ramped down to 25° C. at 5° C./minute. pENTR1 vector digested with FastDigest BbsI (Fermentas), FastAP (Fermentas) and 10× Fast Digest Buffer are used for the ligation reaction. The digest pENTR1 vector was ligated together with the phosphorylated and annealed oligo duplex (dilution 1:200) from the previous step using T4 DNA Ligase and Buffer (NEB). The ligation was incubated at room temperature for at least 1 hour and then transformed and mini-prepped (GeneJET Plasmid Miniprep Kit, Life Technologies). The plasmids were sequenced to confirm the proper insertion.

TABLE 1

| gRNA Name | gRNA Sequence |
|---|---|
| BAX gRNA 1 Oligo 1 | GCGGCGGTGATGGACGGGTCC |
| | SEQ ID NO: 1 |
| BAX gRNA 2 Oligo 1 | GCGGCGGTGATGGACGGGTC |
| | SEQ ID NO: 2 |
| BAX gRNA 3 Oligo 1 | GTCTCGCCGGGTCCGCGCGGG |
| | SEQ ID NO: 3 |
| BAX gRNA 4 Oligo 1 | GCCTCTCGCCGGGTCCGCGC |
| | SEQ ID NO: 4 |
| BAX gRNA 5 Oligo 1 | GCGGACCCGGCGAGAGGCGG |
| | SEQ ID NO: 5 |
| BAX gRNA 6 Oligo 1 | GTCCCGCCGCCGCCTCTCGCC |
| | SEQ ID NO: 6 |
| Lain 1 gRNA 1 Oligo 1 | GTCCTTGCATTGGTGCGCCTC |
| | SEQ ID NO: 7 |
| Lain 1 gRNA 2 Oligo 1 | GCCTGAGGCGCACCAATGCA |
| | SEQ ID NO: 8 |
| Lain 1 gRNA 3 Oligo 1 | GCAGAGTTCTGTCCTTGCAT |
| | SEQ ID NO: 9 |
| Lain 1 gRNA 4 Oligo 1 | GCATTGGTGCGCCTCAGGCC |
| | SEQ ID NO: 10 |
| Lain 1 gRNA 5 Oligo 1 | GCTAGGCCCAGGAGGGCGGTG |
| | SEQ ID NO: 11 |
| Lain 1 gRNA 6 Oligo 1 | GTCAGGCCAGGCTGCACTGCT |
| | SEQ ID NO: 12 |
| BCL2 gRNA 1 Oligo 1 | GCTTCTAGCGCTCGGCACCGG |
| | SEQ ID NO: 13 |
| BCL2 gRNA 2 Oligo 1 | GCAGCGCGGGCTTCTAGCGCT |
| | SEQ ID NO: 14 |
| BCL2 gRNA 3 Oligo 1 | GGGCTTCTAGCGCTCGGCAC |
| | SEQ ID NO: 15 |
| BCL2 gRNA 4 Oligo 1 | GTTCTAGCGCTCGGCACCGGC |
| | SEQ ID NO: 16 |
| BCL2 gRNA 5 Oligo 1 | GAAATGAAGGCAGGACGCGCC |
| | SEQ ID NO: 17 |
| BCL2 gRNA 6 Oligo 1 | GTCATTTATCCAGCAGCTTTT |
| | SEQ ID NO: 18 |
| CD19 gRNA 1 | GAAGCGGGGACTCCCGAGACC |
| | SEQ ID NO: 19 |
| CD19 gRNA 2 | GTTCAACGTCTCTCAACAGAT |
| | SEQ ID NO: 20 |
| CD19 gRNA 3 | GGGGCCTCATGTGGATTCCC |
| | SEQ ID NO: 21 |
| CD19 gRNA 4 | GCTGTGCTGCAGTGCCTCAA |
| | SEQ ID NO: 22 |
| CD19 gRNA 5 | GCTACCTGTGCCAGCCG |
| | SEQ ID NO: 23 |
| CD19 gRNA 6 | GTCTCAGAGGGGGGCCCCGGC |
| | SEQ ID NO: 24 |
| CD19 Cell For | TACCCTCTCTGAGCCTCCAT |
| | SEQ ID NO: 25 |
| CD19 Cell Rev | CCTCTCTCCAGCTCCATTGT |
| | SEQ ID NO: 26 |
| hIGKC gRNA 1 | GGTGGATAACGCCCTCCAAT |
| | SEQ ID NO: 27 |
| hIGKC gRNA 2 | TCAACTGCTCATCAGATGGC |
| | SEQ ID NO: 28 |
| hIGKC gRNA 3 | ATCCACCTTCCACTGTACTT |
| | SEQ ID NO: 29 |
| hIGKC gRNA 4 | ATTCAGCAGGCACACAACAG |
| | SEQ ID NO: 30 |
| hIGKC gRNA 5 | CCTGCTCTGTGACACTCTCC |
| | SEQ ID NO: 31 |
| hIGKC gRNA 6 | TCTCCTGGGAGITACCCGAT |
| | SEQ ID NO: 32 |

Validation of gRNAs

293T cells were plated out at a density of 1×10⁵ cells per well in a 24 well plate. 150 μL of Opti-MEM medium was combined with 1.5 μg of gRNA plasmid, 1.5 μg of Cas9 plasmid and 100 ng of GFP. Another 150 μL of Opti-MEM medium was combined with 5 μL of Lipofectamine 2000 Transfection reagent (Invitorgen, Life Technologies). The solutions were combined together and incubated for 10 minutes to 15 minutes at room temperature. The DNA-lipid complex was added dropwise to one well of the 24 well plate. Cells were incubated for 3 days at 37° C. and then genomic DNA was collected using the GeneJET Genomic DNA Purification Kit (Thermo Scientific). Activity of the gRNAs was quantified by a Surveyor Digest, gel electrophoresis, and densitometry (Guschin et al. Methods Mol Biol. 2010, 649:247-56).

Isolation of Peripheral Blood Mononuclear Cells (PBMCs) from a Leukopak

Human PBMC (Stem Cell Technologies, Vancouver Canada) were diluted 3:1 with chilled 1×PBS. The diluted blood was added dropwise (very slowly) over 15 mL of Lymphoprep (Stem Cell Technologies, Vancouver, Canada) in a 50 mL conical. Cells were spun at 400×g for 25 minutes with no brake. The buffy coat was removed and placed into a new conical. The cells were washed with chilled 1×PBS and spun for 400×g for 10 minutes (with brake). The supernatant was removed, cells resuspended in freeze media, counted and frozen.

### Isolation of CD19⁺ B Cells

PBMCs were thawed and counted; the cell density was adjusted to 1×10⁸ cells/mL, and cells were transferred to a 14 mL polystyrene round-bottom tube. B cells were selected using the EasySep Human CD19 Positive Selection Kit (Stem Cell Technologies, Vancouver, Canada), following manufacturers' protocol. Collected cells were spun at 400×g for 5 minutes and resuspended in growth medium.

### Activation and Stimulation of CD19⁺ B Cells

To stimulate B cells, the isolated CD19⁺ B cells were counted and plated out at a density of 1×10⁶ cells/mL in a 24-well plate. Cells were plated with CD40L and IL-4 following manufacturer's protocol (Miltenyi Biotec Inc, San Diego, Calif.). Cells were incubated for 1 week at 37° C. and then counted using a hemocytometer.

### NEON Transfection of CD19⁺ B Cells

Unstimulated or stimulated B cells were electroporated using the NEON Transfection System (100 μL Kit, ThermoFisher Scientific, Inc., Waltham, Mass.) according to manufacturer instructions except for any variations described below. Cells were counted and resuspended at a density of 1×10⁶ cells in 100 μL of Resuspension Buffer T. 5 μg of GFP plasmid or mRNA or 15 μg Cas9 and 10 μg of plasmid or mRNA gRNA (in molecular water) were added to the cell mixture. Cells were electroporated at 1400 V, 10 ms, 3 pulses. After transfection, cells were plated in a 2 mL culturing media in a 6 well plate.

Unstimulated or stimulated B cells were nucleoporated according to manufacturer instructions using the AMAXA NUCLEOFECTOR and the Human B Cell NUCLEOFECTOR Kit (Lonza Cologne GmbH, Cologne, Germany).

### Flow Cytometry

Electroporated B cells were analyzed by flow cytometry 24 to 72 hours after transfection for expression of GFP. Cells were prepped by washing with chilled 1×PBS with 0.5% FBS and stained with Viability Dye eFlour 780 (eBiosciences, San Diego, Calif.). Cells were analyzed using a LSR II (BD Biosciences, San Jose) and FlowJo v.9.

### Homologous Recombination in CD19+ B Cells

Stimulated CD19⁺ B cells were electroporated using the NEON transfection system (100 μL Kit, ThermoFisher Scientific, Inc., Waltham, Mass.). Cells were counted and resuspended at a density of 1.0-3.0×10⁶ cells in 100 μL of Resuspension Buffer T. 15 μg mRNA Cas9 (TriLink BioTechnologies, San Diego, Calif.), 10 μg mRNA gRNA (TriLink BioTechnologies) and 10 μg of homologous recombination (HR) targeting vector were used for to examine HR. 10 μg of HR targeting vector alone or 15 μg Cas9 with 10 μg mRNA gRNA were used as controls. After electroporation, cells were plated in 2 mL of culturing medium in a 6 well plate. Cells were counted using a Countess II Automated. Cell Counter (ThermoFisher Scientific, Inc., Waltham, Mass.) every, three days to monitor growth under these various conditions. In order to monitor for HR, cells were analyzed by flow cytometry and tested by PCR. For flow cytometry, cells were analyzed once a week for three weeks. B cells were stained with Fixable Viability Dye eFluor 780 (eBiosciences, San Diego). Cells were analyzed using a LSR ii (BD Biosciences, San Jose, Calif.) and FlowJo v.9. To test for HR by PCR, gDNA was isolated from B cells and amplified by PCR using accuprime tact DNA polymerase, high fidelity (ThermoFisher Scientific, Inc., Waltham, Mass.). Primers, shown in Table 2 were designed to both the CCR5 gene and to both ends of the HR targeting vector to look for proper homologous recombination.

TABLE 2

| | | |
|---|---|---|
| 5′ HR Primer | TGC ATG TTC TTT | SEQ ID |
| Forward | GTG GGC TA | NO: 33 |
| 5′ HR Primer | CAC GGC GAC TAC | SEQ ID |
| Reverse | TGC ACT TA | NO: 34 |
| 3′ HR Primer | GGG AGG ATT GGG | SEQ ID |
| Forward | AAG ACA AT | NO: 35 |
| 3′ HR Primer | TGT CTT TIC TCC | SEQ ID |
| Reverse | CCA TAG CAA | NO: 36 |

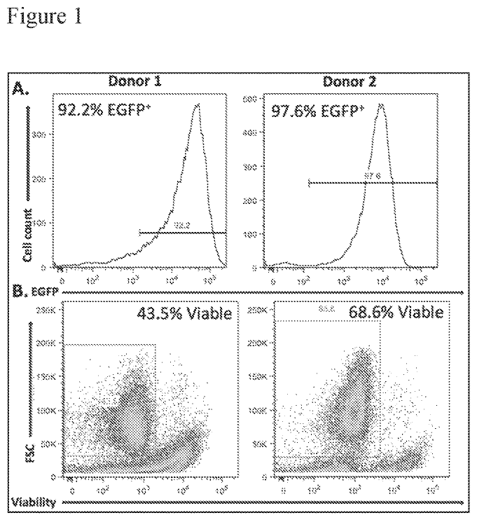

Results are shown in FIGS. 1 to 2.

### Example 2

### Methods

Validation of gRNAs

293T cells were plated out at a density of 1×10⁵ cells per well in a 24 well plate. 150 microliters (μL) of Opti-MEM medium was combined with 1.5 micrograms (μg) of gRNA plasmid, 1.5 μg of Cas9 plasmid, and 100 nanograms (ng) of GFP. Another 150 μL of Opti-MEM medium was combined with 5 μL of Lipofectamine 2000 Transfection reagent (Invitrogen, Carlsbad, Calif.; Life Technologies, Carlsbad, Calif.). The solutions were combined and incubated for 10 to 15 minutes at room temperature. The DNA-lipid complex was added dropwise to one well of the 24 well plate. Cells were incubated for 3 days at 37° C. and then genomic DNA was collected using the GeneJET Genomic DNA Purification Kit (Thermo Fisher Scientific, Waltham, Mass.).

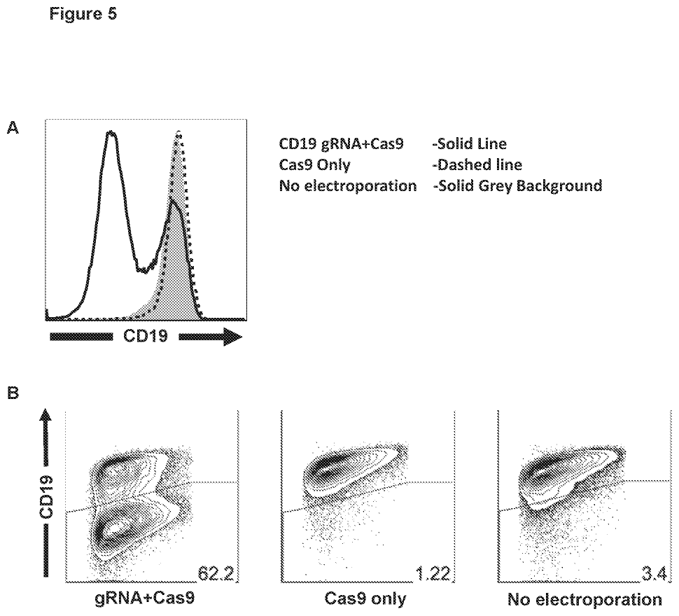

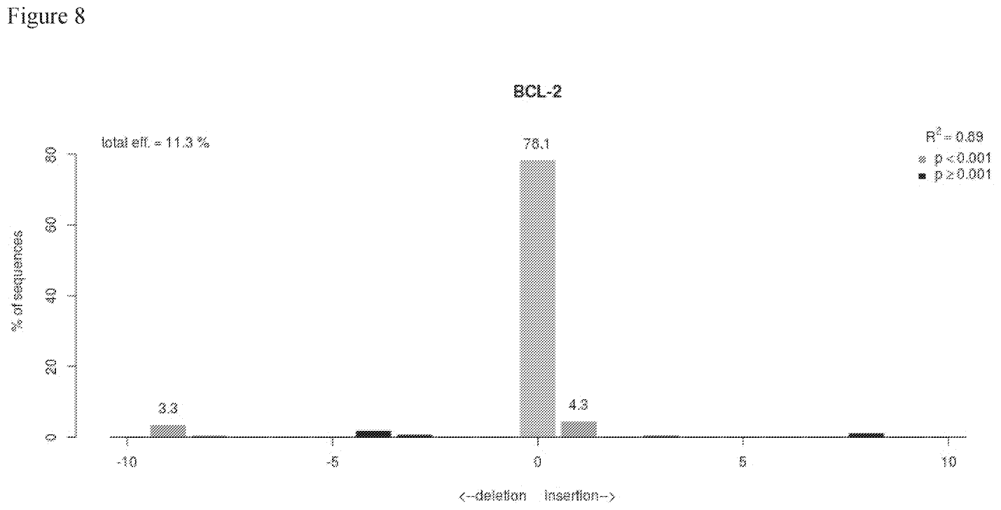

For FIGS. 5 and 8, activity of the gRNAs was quantified by the Tracking of Indels by Decomposition (TIDE) algorithm (available on the world wide web at tide.nki.nl). Briefly, the edited region was amplified by PCR using region-specific primers, and sent to ACGT, Inc. (Wheeling, Ill.) for Sanger Sequencing. Chromatogram files returned from ACGT, Inc. were uploaded to the TIDE website for analysis of editing efficiency.

### Production and Titration of Lentiviral Vectors

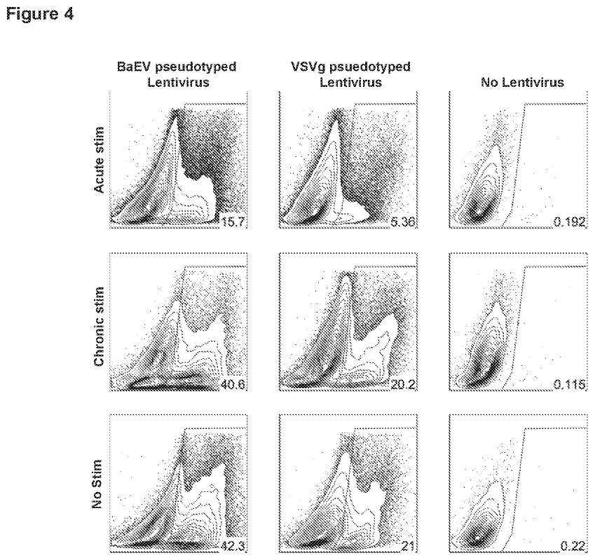

BaEV-psuedotype and VSVg-psuedotype lentiviral vectors were generated by transient transfection of 293T cells using Lipofectamine 2000 Transfection Reagent (Invitrogen, Waltham, Mass.) in accordance with the manufacturer's instructions. 15 μg of BaEV glycoprotein (Fusil et al., Molecular Therapy, 2015, 23(11):1734-47) or 15 μg of VSV glycoprotein were combined with 20 μg of a gagpol packaging plasmid and cargo plasmid to construct BaEV-psuedotype or VSVg-psuedotype viruses, respectively. Eighteen hours after transfection, media was replaced with Dulbecco's Modified Eagle Media (DMEM) containing 10% FBS and 1× penicillin-streptomycin. Viral titers were harvested after 24 hours and filtered to remove cellular debris. Lentiviral titers were determined using a qPCR Lentivirus Titration Kit (Applied Biological Materials Inc., Vancouver, Canada) in accordance with manufacturer's instructions. Results are shown in FIG. 4.

Two additional lentiviral vectors were constructed to expressed either B12 heavy chain and light chain or anti-PE heavy chain and light chain and codon-optimized IDUA (coIDUA) under regulation of a MND promoter (a synthetic promoter that contains the U3 region of a modified MoMuLV LTR with myeloproliferative sarcoma virus enhancer). Co-expression of the heavy chain, the light chain, and the coIDUA was obtained by introducing the P2A peptide sequences. These vectors use alternative splicing of mRNA transcripts to induce the expression of functional BCR in mature naive B cells or functional soluble antibody in plasma cells; thus, the expression of either BCR or soluble antibody is dependent on the maturation state of the B cell. Schematics of the vectors are shown in FIG. 6. Sequences of the vectors are shown in Tables 3 to 8.

### Acute and Chronic Activation of B Cells

Both acute and chronically activated B cells were activated with a B Cell Expansion Kit (Miltenyi Biotec Inc, San Diego, Calif.) which activates B cells via crosslinking of CD40. Acutely activated cells were stimulated for 12 hours before application of the lentiviral constructs; chronically activated cells were activated for 14 days before application of the lentiviral constructs.

### Targeted Knockout of CD19 Expression

CD19 expression was knocked out in primary human B cells using a CRISPR/Cas9 system. Chronically activated B cells were transfected with 1.5 μg of chemically modified mRNA coding for Cas9 protein (TriLink BioTechnologies, San Diego, Calif.) and 1 μg of chemically modified CD19 gRNA 4 oligo 1 (TriLink Biotechnologies) with the NEON Transfection System (1400 volts, 10 ms, 3 pulses). The combination of Cas9 protein and CD19 gRNA creates a double stranded break, which in turn leads to indel formation and frameshift mutations which eliminates gene expression and protein levels.

### Evaluation of CD19 Expression

Five days following electroporation, engineered primary human B cells were stained with APC e-Fluor780 Fixable Viability Dye and anti-CD19 antibody conjugated to BV421 (BioLegend, San Diego, Calif.). Cells were run on a LSRII flow cytometer (BD Biosciences), and data was analyzed using FlowJo Version 9 (Tree Star, Ashland, Oreg.). Results are shown in FIG. 5.

### Testing of Electroporation Conditions in B Cells

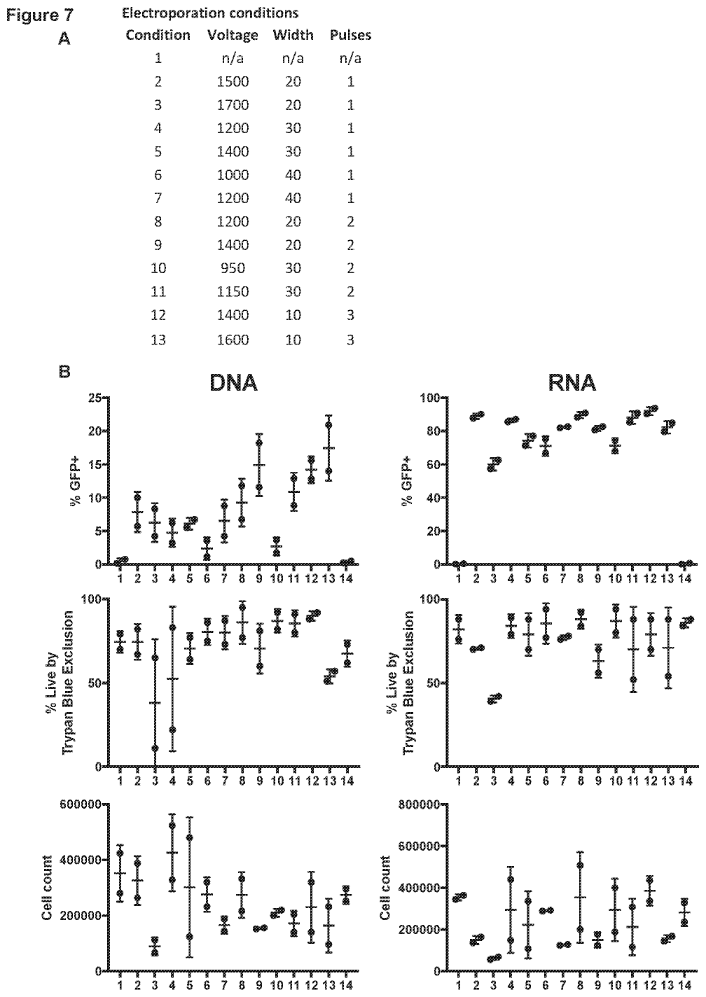

B cells activated for 14 days were electroporated with the NEON transfection under a variety of voltages, widths, and pulse settings (FIG. 7A) with either 1 μg of plasmid DNA or mRNA encoding eGFP. Percent transfection was determined by eGFP expression 2 days post electroporation and measured on a LSRII flow cytometer. Cell counts and cell viability were determined by Trypan Blue exclusion. Results are shown in FIG. 7B.

### Gene Knockout in CD19⁺ B Cells

Stimulated CD19⁺ B cells were electroporated using the NEON Transfection Kit and System (Invitrogen, Carlsbad, Calif.). Gene editing at the BCL2 locus (gRNA 6 Oligo 1) was done using Alt-R CRISPR-Cas9 reagents from Integrated DNA Technologies (Coralville, Iowa). In brief, 1.1 uL of 200 uM Alt-R CRISPR-Cas9 crRNA, 1.1 uL Alt-R tracrRNA, and 2.8 uL nuclease-free duplex buffer were incubated at 95° C. for 5 minutes, then allowed to cool down at room temperature (RT) to form a crRNA:tracrRNA duplex. 0.5 uL of 22 picomolar (pmol) crRNA:tracrRNA duplex and 0.5 uL of 18 pmol Alt-R Cas9 enzyme were incubated at RT for 20 min to form the Alt-R CRISPR-Cas9 system. This Alt-R CRISPR-Cas9 system was combined with Electroporation enhancer (Invitrogen, Carlsbad, Calif.) and then added to 360,000 chronically activated B cells to a final volume was 12 μL in T buffer. A 10 μL pipette tip was used to electroporate the cells at 1400 Volts, 10 ms, 3 pulses. The electroporated cells were cultured for 5 days before gene editing was measured using TIDE analysis as described as above. Results are shown in FIG. 8.

### B Cell Expansion

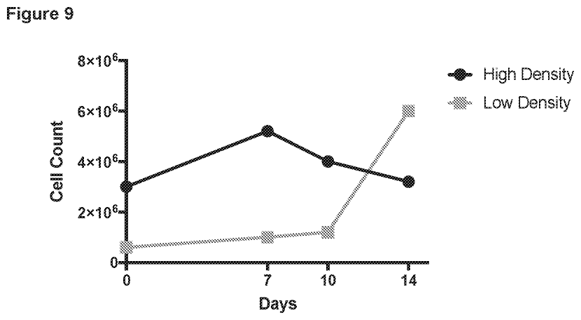

Sorted CD19⁺ B cells were expanded using a B cell expansion kit (Miltenyi Biotec Inc, San Diego, Calif.) in accordance with the manufacturer's protocols for 14 days. Both a low density starting B cell concentration (1×10⁵ cells/mL) and a high density starting B concentration (1×10⁶ cells/mL) were tested. Trypan Blue exclusion was used to determine cell counts at indicated timepoints. Results are shown in FIG. 9.

### IDUA Activity

293T cells were electroporated using NEON System (1400 Volts, 10 ms, 3 pulses) with either GFP mRNA alone (control) or GFP mRNA together with pLL MND B12-IDUA expression plasmid. The pLL MND B12-IDUA expression plasmid includes the expression cassette shown in FIG. 6C.

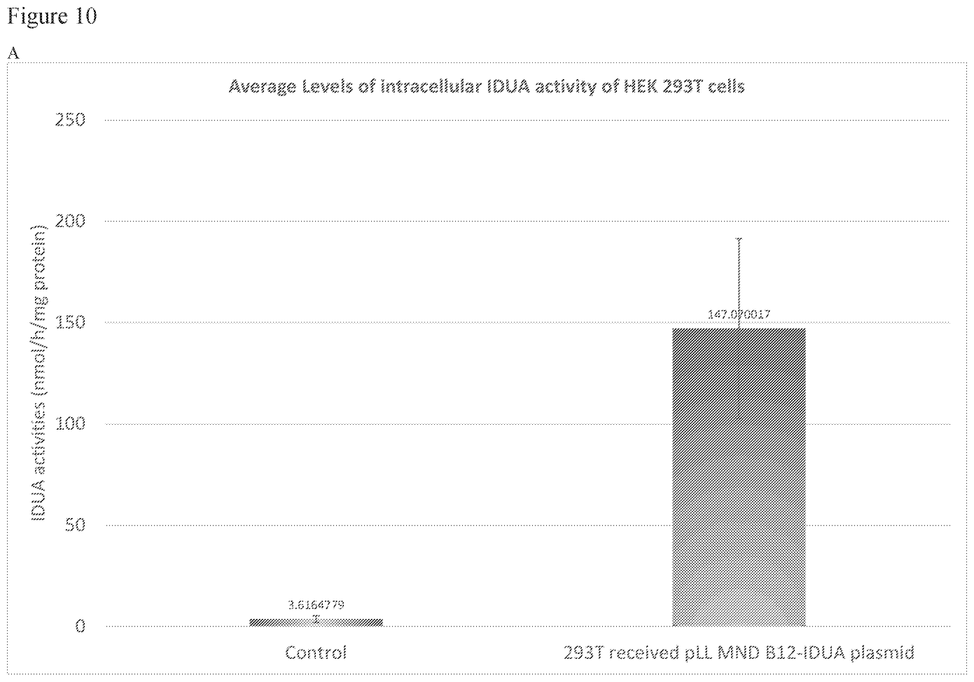

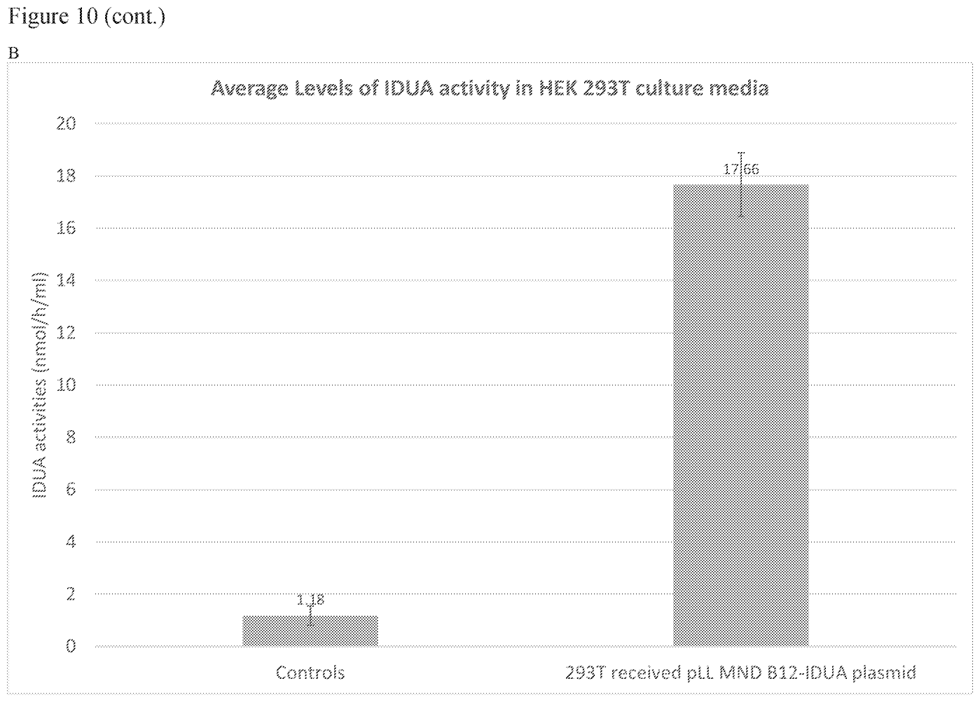

To measure intracellular IDUA activity, the cells were harvested 3 days post electroporation. IDUA activities (nmol/hour/mg protein) were measured using IDUA assay described in Ou et al. 2014, Mol Genet Metab., 2014; 111(2):113-5. The HEK 293T cells that received pLL MND B12-IDUA expression plasmid showed significantly higher intracellular IDUA activities (40-fold higher) than control. Results are shown in FIG. 10A.

To measure IDUA activity in the cell media, media was collected 3 days post electroporation. IDUA activities (nmol/hour/mL) were measured using IDUA assay described in Ou et al. 2014, Mol Genet Metab., 2014; 111(2):113-5. The culture media that contained HEK 293T cells received pLL MND B12-IDUA expression plasmid showed significantly higher IDUA activities (14-fold higher) than control. Results are shown in FIG. 10B.

The complete disclosure of all patents, patent applications, and publications, and electronically available material (including, for instance, nucleotide sequence submissions in, e.g., GenBank and RefSeq, and amino acid sequence submissions in, e.g., SwissProt, PR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

TABLE 3
Coding sequence for Anti-PE heavy chain/light chain/coIDUA lentiviral co-
expression cassette (SEQ ID NO: 37). Long terminal repeats (LTRs) are in
bold text starting at the *. MND promoter is in bold text starting at
the #. Anti-PE heavy chain (†), light chain (Δ), and coIDUA ( [symbol] ) are
in bold starting at the indicated symbols. P2As are italicized.

*
GGGTCTCTCTGGTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTGGTAACTAGAG
ATCCCTCAGACCCTTTTAGTCAGTGTGGAAAATCTCTAGCAGTGGCGCCCGAACAGGGACTTGAAAGCGA
AAGGGAAACCAGAGGAGCTCTCTCGACGCAGGACTCGGCTTGCTGAAGCGCGCACGGCAAGAGGCGAGGG
GCGGCGACTGGTGAGTACGCCAAAAATTTTGACTAGCGGAGGCTAGAAGGAGAGAGATGGGTGCGAGAGC
GTCAGTATTAAGCGGGGGAGAATTAGATCGCGATGGGAAAAAATTCGGTTAAGGCCAGGGGGAAAGAAAA
AATATAAATTAAAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAGGATCAGAAGAA
CTTAGATCATTATATAATACAGTAGCAACCCTCTATTGTGTGCATCAAAGGATAGAGATAAAAGACACCA
AGGAAGCTTTAGACAAGATAGAGGAAGAGCAAAACAAAAGTAAGACCACCGCACAGCAAGCGGCCGGCCG
CGCTGATCTTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAGAAAAAAGA
GCAGTGGGAATAGGAGCTTTGTTCCTTGGGTTCTTGGGAGCAGCAGGAAGCACTATGGGCGCAGCGTCAA
TGACGCTGACGGTACAGGCCAGACAATTATTGTCTGGTATAGTGCAGCAGCAGAACAATTTGCTGAGGGC
TATTGAGGCGCAACAGCATCTGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTGCTCTGGAAAACTCATTTGCA
CCACTGCTGTGCCTTGGAATGCTAGTTGGAGTAATAAATCTCTGGAACAGATTTGGAATCACACGACCTG
GATGGAGTGGGACAGAGAAATTAACAATTACACAAGCTTAATACACTCCTTAATTGAAGAATCGCAAAAC
CAGCAAGAAAAGAATGAACAAGAATTATTGGAATTAGATAAATGGGCAAGTTTGTGGAATTGGTTTAACA
TAACAAATTGGCTGTGGTATATAAAATTATTCATAATGATAGTAGGAGGCTTGGTAGGTTTAAGAATAGT
TTTTGCTGTACTTTCTATAGTGAATAGAGTTAGGCAGGGATATTCACCATTATCGTTTCAGACCCACCTC
CCAACCCCGAGGGGACCCGACAGGCCCGAAGGAATAGAAGAAGAAGGTGGAGAGAGAGACAGAGACAGAT
CCATTCGATTAGTGAACGGATCGGCACTGCGTGCGCCAATTCTGCAGACAAATGGCAGTATTCATCCACA
ATTTTAAAAGAAAAGGGGGGATTGGGGGGTACAGTGCAGGGGAAAGAATAGTAGACATAATAGCAACAGA
CATACAAACTAAAGAATTACAAAAACAAATTACAAAAATTCAAAATTTTCGGGTTTATTACAGGGACAGC
AGAGATCCAGTTTGGTTAGTACCGGGCCCGCTCTAGAGATCCGACGCCGCCATCTCTAGGCCCGCGCCGG
CCCCCTCGCACAGACTTGTGGGAGAAGCTCGGCTACTCCCCTGCCCCGGTTAATTTGCATATAATATTTC
CTAGTAACTATAGAGGCTTAATGTGCGATAAAAGACAGATAATCTGTTCTTTTTAATACTAGCTACATTT
TACATGATAGGCTTGGATTTCTATAAGAGATACAAATACTAAATTATTATTTTAAAAAACAGCACAAAAG
GAAACTCACCCTAACTGTAAAGTAATTGTGTGTTTTGAGACTATAAATATCCCTTGGAGAAAAGCCTTGT
TAACGCGCGGTGACCCTCGAGGTCGACGGTATCGATAAGCTCGCTTCACGAGATTCCAGCAGGTCGAGGG
ACCTAATAACTTCGTATAGCATACATTATACGAAGTTATATTAAGGGTTCCAAGCTTAAGCGGCCGCGTG
GATAACCGTATTACCGCCATGCAT [symbol] TCGATCACGAGACTAGCCTCGAGAAGCTTGATATCGAATTCCACG
GGGTTGGACGCGTCTTAATTAAGGATCCAAGGTCAGGAACAGAGAAACAGGAGAATATGGGCCAAACAGG
ATATCTGTGGTAAGCAGTTCCTGCCCCGGCTCAGGGCCAAGAACAGTTGGAACAGCAGAATATGGGCCAA
ACAGGATATCTGTGGTAAGCAGTTCCTGCCCCGGCTCAGGGCCAAGAACAGATGGTCCCCAGATGCGGTC
CCGCCCTCAGCAGTTTCTAGAGAACCATCAGATGTTTCCAGGGTGCCCCAAGGACCTGAAATGACCCTGT
GCCTTATTTGAACTAACCAATCAGTTCGCTTCTCGCTTCTGTTCGCGCGCTTCTGCTCCCCGAGCTCTAT
ATAAGCAGAGCTCGTTTAGTGAACCGTCAGATCGCCTGGAGACGCCATCCACGCTGTTTTGACCTCCATA
GAAGACACCGACTCTAGAGGATCGATCCCCCGGGCTGCAGGAATTCAAGCGAGAAGACAAGGGCAGAAAG
CACCGCTAGCCAAGTTTGTACAAAAAAGCAGGCTGCCACC [symbol] TGGAAGTACAGCTGGAACAGAGTGGTGCA
GAGTTGGCAAGACCAGGGGCCAGCGTGAAACTCTCTTGCAAGGCGAGTGGATATACCTTTACAAGTTACG
GTATTTCTTGGGTAAAACAGAGGACGGGGCAGGGTCTGGAATGGATAGGTGAAATTTACCCTAAATCCGG
CAATACATACTATAATGAAAAATTCAAAGGACGAGCGACACTTACCGCTGATAAGTCTAGTTCCACTGCG
TATATGGAACTCCGAAGCCTTACCAGCGAGGACTCCACAGTGTATTTTTGCGCGCGACAGGGGTACTATG
CAAATTCACAATTCACGTACTGGGGTCAAGGGACACTTGTAACAGTAAGCGCAGCCAGTACTAAGGGGCC
GAGCGTCTTTCCTCTGGCTCCTAGCTCAAAATCCACATCCGGCGGTACCGCAGCGCTCGGATGTCTTGTT
AAAGATTATTTTCCAGAACCTGTCACAGTCAGTTGGAATAGCGGCGCTTTGACTTCCGGGGTCCATACGT
TTCCGGCGGTGCTGCAATCATCCGGCCTCTATTCCCTCAGCTCTGTTGTTACAGTGCCGAGCTCCTCTCT
GGGCACTCAGACTTATATTTGCAACGTAAACCACAAGCCATCTAACACAAAAGTGGATAAGAAGGTAGAA
CCTAAGAGCTGTGACAAGACGCATACATGCCCGCCGTGCCCTGCCCCAGAACTGCTCGGAGGACCATCCG
TATTCCTCTTTCCCCCCAAGCCCAAAGACACCCTGATGATTTCAAGAACGCCGGAGGTGACGTGTGTTGT
TGTAGATGTCTCCCACGAAGACCCCGAAGTAAAGTTTAATTGGTATGTTGATGGCGTGGAAGTCCACAAC
GCCAAAACCAAGCCTAGAGAAGAGCAGTACAATTCAACATATAGAGTAGTGAGTGTATTGACTGTCCTTC
ACCAGGACTGGCTCAATGGTAAGGAGTATAAGTGCAAGGTGAGCAACAAGGCGCTCCCCGCTCCTATAGA
AAAAACTATCAGTAAAGCAAAAGGTCAACCGAGAGAGCCTCAGGTCTATACACTCCCCCCCAGCCGGGAT
GAACTGACGAAAAATCAGGTCTCACTTACGTGTCTGGTTAAAGGTTTCTATCCGTCTGATATTGCGGTGG
AATGGGAATCAAATGGTCAGCCAGAAAACAATTACAAAACGACGCCCCCAGTGCTGGACTCCGATGGCTC
TTTCTTCCTTTATTCCAAACTTACAGTAGATAAGTCAAGATGGCAACAAGGGAACGTGTTTAGTTGCAGC
GTTATGCATGAAGCACTCCATAATCACTATACCCAAAAGAGCTTGTCCCTTTCCCCAGAGCTTCAACTGG
AGGAGTCTTGCGCAGAGGCACAGGATGGTGAGCTGGATGGGTTGTGGACGACAATAACCATCTTTATAAC
ACTTTTCCTTCTGTCTGTCTGCTACAGCGCTACTGTTACCTTTTTCAAAGTTAAGTGGATATTCAGTTCA
GTTGTCGATTTGAAACAGACTATCATCCCAGACTATCGGAACATGATAGGCCAAGGCGCCAGAGCCAAAA
GAAGTGGTTCTGGCGCGACGAATTTTAGTTTGCTTAAGCAAGCCGGAGATGTGGAGGAAAATCCTGGACC
G [symbol] TGGACATTGTGTTGACCCAGACACCGGCTATTATGTCTGCCTCACTCGGCGAAAGAGTAACGATGACT
TGCACCGCGAGTTCTAGCGTCAGCTCTAGCTACCTTCATTGGTTTCAGCAAAAACCCGGGTCAAGTCCAA
AGCTCTGGATCTACTCTACATCAAATCTGGCATCCGGTGTTCCTGGTAGGTTCTCCGGCTCCGGCAGCGA
AACTTCTTATTCCCTGATTATTGGCTCCCTCGAGACAGAAGATGCCGCAACCTACTACTGTCACCAATAT
CACCGGAGCCCGCCCACATTTGGAGGAGGAACTAAATTGGAAATCAAGACAGTTGCAGCGCCTAGCGTAT
TTATTTTCCCACCCTCAGATGAGCAACTGAAATCCGGGACCGCATCTGTTGTCTGTCTGCTGAACAACTT
CTATCCCCGAGAGGCCAAAGTTCAGTGGAAAGTGGACAATGCGCTTCAATCTGGTAACTCTCAAGAATCC
GTCACTGAGCAGGATTCTAAGGATTCTACATATAGTCTTTCATCAACCCTTACACTTAGTAAGGCGGACT
ATGAAAAACACAAAGTGTACGCATGTGAGGTAACTCACCAGGGGCTGCGCTCCCCGGTTACCAAAAGTTT
TAATCGAGGGGAGTGCAGAGCCAAAAGAAGTGGTTCTGGCGCGACGAATTTTAGTTTGCTTAAGCAAGCC
GGAGATGTGGAGGAAAATCCTGGACCG [symbol] GAGGCCGCTCAGACCGAGGGCTGCTCTTCTCGCCCTTCTTG
CATCCTTGCTGGCCGCCCCCCCTGTCGCACCCGCTGAAGCCCCACACCTCGTACACGTGGATGCTGCGCG
CGCGCTGTGGCCATTGCGCCGATTTTGGAGGAGCACAGGGTTCTGTCCTCCCTTGCCCCACTCTCAAGCG
GACCAATACGTCCTCAGTTGGGACCAGCAACTTAACCTGGCCTACGTTGGGGCAGTGCCACATAGAGGCA
TAAAACAGGTTCGAACCCACTGGCTGCTGGAGTTGGTAACTACACGCGGCAGTACCGGTCGAGGTCTCTC
ATACAATTTTACTCATCTCGACGGATACCTTGACTTGCTGAGGGAAAACCAACTGTTGCCCGGTTTCGAA
CTGATGGGTAGCGCATCCGGCCACTTTACTGATTTCGAGGACAAACAACAAGTTTTTGAATGGAAGGATT
TGGTAAGCAGCCTGGCGAGAAGATATATCGGCCGATACGGGCTCGCGCACGTGTCTAAATGGAATTTTGA
AACGTGGAACGAACCTGACCATCACGATTTTGACAACGTCAGCATGACAATGCAGGGGTTCCTTAACTAT
TACGACGCGTGTTCTGAAGGTTTGAGAGCGGCTTCCCCTGCACTCCGCTTGGGAGGGCCCGGGGACAGCT
TCCATACCCCCCCGCGGTCACCGTTGTCATGGGGCCTCTTGCGGCACTGTCATGATGGCACAAATTTTTT
TACGGGAGAAGCCGGTGTGAGACTGGATTACATTAGTCTCCACCGAAAGGGAGCCCGCTCAAGCATTAGT
ATCCTGGAACAAGAGAAAGTGGTTGCTCAGCAGATTCGGCAATTGTTCCCAAAATTCGCTGACACGCCAA
TATATAACGATGAAGCTGACCCCTTGGTGGGGTGGAGTCTTCCTCAACCTTGGCGCGCCGATGTAACGTA
CGCTGCAATGGTGGTAAAGGTCATAGCCCAACATCAGAACCTGCTCCTCGCCAATACGACAAGTGCCTTT
CCCTACGCGCTTTTGTCCAATGATAACGCGTTTCTGAGCTACCACCCTCACCCGTTTGCTCAGCGAACAT
TGACCGCTAGGTTCCAAGTGAATAATACACGACCGCCCCACGTCCAACTCTTGCGCAAACCTGTTCTTAC
CGCGATGGGGCTCCTCGCTCTTCTCGACGAAGAGCAACTCTGGGCAGAAGTATCCCAAGCGGGCACTGTG
CTGGACTCCAACCATACTGTAGGCGTGCTGGCCTCCGCCCACCGCCCCCAGGGACCAGCAGACGCGTGGA
GGGCTGCGGTTCTGATATATGCCAGTGACGACACAAGGGCGCACCCGAATAGGTCAGTCGCTGTGACTTT
GCGGCTGAGAGGGGTCCCACCGGGTCCTGGACTGGTTTATGTAACGAGATACCTGGACAATGGTCTTTGC
TCCCCCGATGGCGAGTGGCGGAGGCTGGGGAGGCCCGTATTTCCGACTGCTGAACAGTTTCGACGAATGC
GAGCAGCTGAAGATCCCGTAGCAGCAGCACCACGCCCTCTTCCTGCTGGAGGTAGACTTACGTTGCGACC
GGCCCTCCGCCTTCCTTCACTGCTCCTCGTACATGTCTGCGCACGCCCCGAGAAGCCACCAGGCCAGGTC
ACTCGACTCCGAGCCCTTCCGCTCACCCAAGGACAGCTCGTCCTTGTCTGGTCTGACGAGCATGTCGGTA
GCAAATGCCTTTGGACGTACGAGATACAGTTTAGCCAGGACGGTAAGGCATATACTCCTGTTAGTAGGAA
GCCATCTACTTTTAACCTTTTTGTATTCAGTCCCGACACGGGTGCTGTCAGCGGGTCTTACCGGGTCCGC
GCCCTGGATTATTGGGCCAGACCCGGACCTTTCTCCGATCCTGTCCCCTATCTCGAGGTGCCCGTGCCAA
GGGGGCCTCCCAGCCCCGGCAATCCATAATGAACCCAGCTTTCTTGTACAAAGTGGTTGTACAAGTAGGA
ATTCGTCGAGGGACCTAATAACTTCGTATAGCATACATTATACGAAGTTATACATGTTTAAGGGTTCCGG
TTCCACTAGGTACAATTCGATATCAAGCTTATCGATAATCAACCTCTGGATTACAAAATTTGTGAAAGAT
TGACTGGTATTCTTAACTATGTTGCTCCTTTTACGCTATGTGGATACGCTGCTTTAATGCCTTTGTATCA
TGCTATTGCTTCCCGTATGGCTTTCATTTTCTCCTCCTTGTATAAATCCTGGTTGCTGTCTCTTTATGAG
GAGTTGTGGCCCGTTGTCAGGCAACGTGGCGTGGTGTGCACTGTGTTTGCTGACGCAACCCCCACTGGTT
GGGGCATTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTCCGTGGTG
TTGTCGGGGAAATCATCGTCCTTTCCTTGGCTGCTCGCCTGTGTTGCCACCTGGATTCTGCGCGGGACGT
CCTTCTGCTACGTCCCTTCGGCCCTCAATCCAGCGGACCTTCCTTCCCGCGGCCTGCTGCCGGCTCTGCG
GCCTCTTCCGCGTCTTCGCCTTCGCCCTCAGACGAGTCGGATCTCCCTTTGGGCCGCCTCCCCGCATCGA
TACCGTCGACCTCGATCGAGACCTAGAAAAACATGGAGCAATCACAAGTAGCAATACAGCAGCTACCAAT
GCTGATTGTGCCTGGCTAGAAGCACAAGAGGAGGAGGAGGTGGGTTTTCCAGTCACACCTCAGGTACCTT
TAAGACCAATGACTTACAAGGCAGCTGTAGATCTTAGCCACTTTTTAAAAGAAAAGGGGGGACTGGAAGG
GCTAATTCACTCCCAACGAAGACAAGATATCCTTGATCTGTGGATCTACCACACACAAGGCTACTTCCCT
GATTGGCAGAACTACACACCAGGGCCAGGGATCAGATATCCACTGACCTTTGGATGGTGCTACAAGCTAG
TACCAGTTGAGCAAGAGAAGGTAGAAGAAGCCAATGAAGGAGAGAACACCCGCTTGTTACACCCTGTGAG
CCTGCATGGGATGGATGACCCGGAGAGAGAAGTATTAGAGTGGAGGTTTGACAGCCGCCTAGCATTTCAT
CACATGGCCCGAGAGCTGCATCCGGACTGTAC [symbol] GGTCTCTCTGGTTAGACCAGATCTGAGCCTGGGAGC
TCTCTGGCTAACTAGGGAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTCAGTGTGGAAAATCTCT
AGCA

TABLE 4
Coding sequence for B12 heavy chain/light chain/coIDUA lentiviral co-
expression cassette (SEQ ID NO: 38). Long terminal repeats (LTRs) are
in bold text starting at the *. MND promoter is in bold text starting
at the #. B12 heavy chain (†), light chain (Δ), and
coIDUA ( [symbol] ) are in bold at the indicated symbols.
P2As are italicized.

*
GGGTCTCTCTGGTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTGGTAACTAGAG
ATCCCTCAGACCCTTTTAGTCAGTGTGGAAAATCTCTAGCAGTGGCGCCCGAACAGGGACTTGAAAGCGA
AAGGGAAACCAGAGGAGCTCTCTCGACGCAGGACTCGGCTTGCTGAAGCGCGCACGGCAAGAGGCGAGGG
GCGGCGACTGGTGAGTACGCCAAAAATTTTGACTAGCGGAGGCTAGAAGGAGAGAGATGGGTGCGAGAGC
GTCAGTATTAAGCGGGGGAGAATTAGATCGCGATGGGAAAAAATTCGGTTAAGGCCAGGGGGAAAGAAAA
AATATAAATTAAAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAGGATCAGAAGAA
CTTAGATCATTATATAATACAGTAGCAACCCTCTATTGTGTGCATCAAAGGATAGAGATAAAAGACACCA
AGGAAGCTTTAGACAAGATAGAGGAAGAGCAAAACAAAAGTAAGACCACCGCACAGCAAGCGGCCGGCCG
CGCTGATCTTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAGAAAAAAGA
GCAGTGGGAATAGGAGCTTTGTTCCTTGGGTTCTTGGGAGCAGCAGGAAGCACTATGGGCGCAGCGTCAA
TGACGCTGACGGTACAGGCCAGACAATTATTGTCTGGTATAGTGCAGCAGCAGAACAATTTGCTGAGGGC
TATTGAGGCGCAACAGCATCTGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTGCTCTGGAAAACTCATTTGCA
CCACTGCTGTGCCTTGGAATGCTAGTTGGAGTAATAAATCTCTGGAACAGATTTGGAATCACACGACCTG
GATGGAGTGGGACAGAGAAATTAACAATTACACAAGCTTAATACACTCCTTAATTGAAGAATCGCAAAAC
CAGCAAGAAAAGAATGAACAAGAATTATTGGAATTAGATAAATGGGCAAGTTTGTGGAATTGGTTTAACA
TAACAAATTGGCTGTGGTATATAAAATTATTCATAATGATAGTAGGAGGCTTGGTAGGTTTAAGAATAGT
TTTTGCTGTACTTTCTATAGTGAATAGAGTTAGGCAGGGATATTCACCATTATCGTTTCAGACCCACCTC
CCAACCCCGAGGGGACCCGACAGGCCCGAAGGAATAGAAGAAGAAGGTGGAGAGAGAGACAGAGACAGAT
CCATTCGATTAGTGAACGGATCGGCACTGCGTGCGCCAATTCTGCAGACAAATGGCAGTATTCATCCACA
ATTTTAAAAGAAAAGGGGGGATTGGGGGGTACAGTGCAGGGGAAAGAATAGTAGACATAATAGCAACAGA
CATACAAACTAAAGAATTACAAAAACAAATTACAAAAATTCAAAATTTTCGGGTTTATTACAGGGACAGC
AGAGATCCAGTTTGGTTAGTACCGGGCCCGCTCTAGAGATCCGACGCCGCCATCTCTAGGCCCGCGCCGG
CCCCCTCGCACAGACTTGTGGGAGAAGCTCGGCTACTCCCCTGCCCCGGTTAATTTGCATATAATATTTC
CTAGTAACTATAGAGGCTTAATGTGCGATAAAAGACAGATAATCTGTTCTTTTTAATACTAGCTACATTT
TACATGATAGGCTTGGATTTCTATAAGAGATACAAATACTAAATTATTATTTTAAAAAACAGCACAAAAG
GAAACTCACCCTAACTGTAAAGTAATTGTGTGTTTTGAGACTATAAATATCCCTTGGAGAAAAGCCTTGT
TAACGCGCGGTGACCCTCGAGGTCGACGGTATCGATAAGCTCGCTTCACGAGATTCCAGCAGGTCGAGGG
ACCTAATAACTTCGTATAGCATACATTATACGAAGTTATATTAAGGGTTCCAAGCTTAAGCGGCCGCGTG
GATAACCGTATTACCGCCATGCAT [symbol] TCGATCACGAGACTAGCCTCGAGAAGCTTGATATCGAATTCCACG
GGGTTGGACGCGTCTTAATTAAGGATCCAAGGTCAGGAACAGAGAAACAGGAGAATATGGGCCAAACAGG
ATATCTGTGGTAAGCAGTTCCTGCCCCGGCTCAGGGCCAAGAACAGTTGGAACAGCAGAATATGGGCCAA
ACAGGATATCTGTGGTAAGCAGTTCCTGCCCCGGCTCAGGGCCAAGAACAGATGGTCCCCAGATGCGGTC
CCGCCCTCAGCAGTTTCTAGAGAACCATCAGATGTTTCCAGGGTGCCCCAAGGACCTGAAATGACCCTGT
GCCTTATTTGAACTAACCAATCAGTTCGCTTCTCGCTTCTGTTCGCGCGCTTCTGCTCCCCGAGCTCTAT
ATAAGCAGAGCTCGTTTAGTGAACCGTCAGATCGCCTGGAGACGCCATCCACGCTGTTTTGACCTCCATA
GAAGACACCGACTCTAGAGGATCGATCCCCCGGGCTGCAGGAATTCAAGCGAGAAGACAAGGGCAGAAAG
CACCGCTAGCCAAGTTTGTACAAAAAAGCAGGCTGCCACC [symbol] TGCAAGTCCAACTGGTACAATCCGGAGCT
GAAGTGAAAAAACCAGGGGCATCCGTGAAAGTCTCATGCCAAGCCTCCGGTTACCGATTTAGTAACTTTG
TGATACACTGGGTTCGGCAAGCCCCAGGACAAAGATTCGAGTGGATGGGCTGGATAAACCCATATAATGG
TAACAAAGAATTCTCAGCTAAATTCCAAGACCGGGTAACATTTACGGCGGATACTTCAGCTAACACTGCA
TACATGGAACTTCGCAGCTTGAGGTCCGCGGACACAGCCGTATATTACTGCGCGAGGGTTGGCCCATATA
GCTGGGACGACAGTCCGCAGGACAATTATTACATGGACGTGTGGGGGAAGGGTACGACCGTAATAGTCAG
TAGTGCGTCCACTAAAGGTCCCTCTGTCTTCCCATTGGCACCGAGTTCTAAGTCTACGTCTGGTGGCACA
GCTGCACTGGGCTGTCTGGTCAAAGACTATTTCCCCGAACCAGTTACGGTTAGTTGGAACAGCGGAGCCC
TGACAAGCGGAGTGCACACGTTTCCGGCGGTTCTTCAATCTAGTGGACTCTATAGCCTCTCATCCGTAGT
AACGGTTCCATCCTCATCTCTTGGAACGCAAACGTATATTTGTAACGTTAATCATAAGCCTTCTAATACT
AAGGTTGACAAAAAGGTAGAGCCTAAATCTTGCGATAAGACGCACACCTGCCCGCCCTGTCCTGCACCCG
AACTTTTGGGGGGTCCATCAGTTTTCCTGTTTCCTCCAAAGCCAAAAGATACTCTGATGATCTCCAGAAC
GCCAGAGGTAACCTGCGTCGTAGTCGATGTTTCTCACGAAGACCCTGAAGTGAAATTTAACTGGTACGTA
GATGGTGTGGAAGTGCACAATGCGAAAACTAAACCGCGCGAAGAACAGTACAACTCTACTTACCGAGTCG
TGTCCGTCCTCACGGTCCTCCATCAGGACTGGCTTAACGGAAAAGAATACAAATGTAAAGTCAGCAACAA
AGCGCTCCCAGCGCCCATAGAAAAAACGATTAGTAAGGCGAAAGGTCAACCTAGAGAGCCACAGGTATAC
ACTCTCCCACCTTCACGGGACGAACTTACGAAAAACCAGGTTTCACTCACATGTTTGGTGAAAGGATTTT
ATCCTAGCGATATTGCTGTAGAATGGGAGAGTAACGGGCAACCTGAGAACAATTATAAGACAACACCTCC
TGTATTGGATTCAGACGGTAGTTTTTTCTTGTATAGCAAATTGACTGTTGACAAGTCCCGGTGGCAACAG
GGGAATGTCTTCTCCTGCTCTGTCATGCATGAAGCCCTTCACAACCATTACACCCAGAAAAGTTTGTCCC
TCTCTCCCGAACTTCAGCTGGAAGAGTCCTGCGCCGAAGCACAAGATGGAGAACTCGATGGCCTGTGGAC
GACCATAACTATCTTCATCACTCTCTTCTTGCTGAGTGTGTGCTACAGCGCCACCGTTACTTTTTTCAAG
GTAAAATGGATATTCTCCAGCGTGGTCGATCTCAAACAGACAATCATTCCGGATTACAGAAACATGATTG
GGCAGGGCGCTAGAGCCAAAAGAAGTGGTTCTGGCGCGACGAATTTTAGTTTGCTTAAGCAAGCCGGAGA
TGTGGAGGAAAATCCTGGACC [symbol] TGGAAATTGTGTTGACTCAGAGCCCCGGAACTCTGTCCCTCAGTCCC
GGCGAAAGGGCAACATTCAGTTGTAGGTCTTCCCATTCAATTCGAAGTAGGCGCGTTGCGTGGTACCAAC
ACAAACCCGGCCAAGCTCCACGCCTGGTGATCCACGGGGTGTCAAATAGGGCATCTGGGATCTCCGATAG
GTTTTCAGGGTCAGGATCAGGTACGGATTTCACCCTTACCATCACACGGGTTGAACCCGAGGATTTCGCC
CTTTATTACTGCCAGGTATATGGGGCGAGCAGTTATACGTTTGGTCAGGGGACCAAGCTCGAGCGAAAAC
GGACCGTCGCGGCCCCAAGCGTCTTTATCTTCCCACCCTCTGACGAACAGCTCAAATCCGGTACAGCTTC
TGTAGTTTGCTTGCTCAACAACTTTTACCCACGGGAGGCGAAAGTTCAATGGAAAGTGGATAATGCCCTT
CAATCCGGGAACAGCCAAGAAAGTGTGACGGAACAGGACAGCAAAGACTCTACCTATTCACTCAGTAGCA
CTCTGACTTTGTCCAAGGCCGATTATGAAAAACATAAGGTTTACGCGTGCGAGGTTACACACCAAGGTCT
GCGAAGCCCAGTAACAAAATCCTTCAATAGGGGTGAGTGCAGAGCCAAAAGAAGTGGTTCTGGCGCGACG
AATTTTAGTTTGCTTAAGCAAGCCGGAGATGTGGAGGAAAATCCTGGACCG [symbol] GAGGCCGCTCAGACCGA
GGGCTGCTCTTCTCGCCCTTCTTGCATCCTTGCTGGCCGCCCCCCCTGTCGCACCCGCTGAAGCCCCACA
CCTCGTACACGTGGATGCTGCGCGCGCGCTGTGGCCATTGCGCCGATTTTGGAGGAGCACAGGGTTCTGT
CCTCCCTTGCCCCACTCTCAAGCGGACCAATACGTCCTCAGTTGGGACCAGCAACTTAACCTGGCCTACG
TTGGGGCAGTGCCACATAGAGGCATAAAACAGGTTCGAACCCACTGGCTGCTGGAGTTGGTAACTACACG
CGGCAGTACCGGTCGAGGTCTCTCATACAATTTTACTCATCTCGACGGATACCTTGACTTGCTGAGGGAA
AACCAACTGTTGCCCGGTTTCGAACTGATGGGTAGCGCATCCGGCCACTTTACTGATTTCGAGGACAAAC
AACAAGTTTTTGAATGGAAGGATTTGGTAAGCAGCCTGGCGAGAAGATATATCGGCCGATACGGGCTCGC
GCACGTGTCTAAATGGAATTTTGAAACGTGGAACGAACCTGACCATCACGATTTTGACAACGTCAGCATG
ACAATGCAGGGGTTCCTTAACTATTACGACGCGTGTTCTGAAGGTTTGAGAGCGGCTTCCCCTGCACTCC
GCTTGGGAGGGCCCGGGGACAGCTTCCATACCCCCCCGCGGTCACCGTTGTCATGGGGCCTCTTGCGGCA
CTGTCATGATGGCACAAATTTTTTTACGGGAGAAGCCGGTGTGAGACTGGATTACATTAGTCTCCACCGA
AAGGGAGCCCGCTCAAGCATTAGTATCCTGGAACAAGAGAAAGTGGTTGCTCAGCAGATTCGGCAATTGT
TCCCAAAATTCGCTGACACGCCAATATATAACGATGAAGCTGACCCCTTGGTGGGGTGGAGTCTTCCTCA
ACCTTGGCGCGCCGATGTAACGTACGCTGCAATGGTGGTAAAGGTCATAGCCCAACATCAGAACCTGCTC
CTCGCCAATACGACAAGTGCCTTTCCCTACGCGCTTTTGTCCAATGATAACGCGTTTCTGAGCTACCACC
CTCACCCGTTTGCTCAGCGAACATTGACCGCTAGGTTCCAAGTGAATAATACACGACCGCCCCACGTCCA
ACTCTTGCGCAAACCTGTTCTTACCGCGATGGGGCTCCTCGCTCTTCTCGACGAAGAGCAACTCTGGGCA
GAAGTATCCCAAGCGGGCACTGTGCTGGACTCCAACCATACTGTAGGCGTGCTGGCCTCCGCCCACCGCC
CCCAGGGACCAGCAGACGCGTGGAGGGCTGCGGTTCTGATATATGCCAGTGACGACACAAGGGCGCACCC
GAATAGGTCAGTCGCTGTGACTTTGCGGCTGAGAGGGGTCCCACCGGGTCCTGGACTGGTTTATGTAACG
AGATACCTGGACAATGGTCTTTGCTCCCCCGATGGCGAGTGGCGGAGGCTGGGGAGGCCCGTATTTCCGA
CTGCTGAACAGTTTCGACGAATGCGAGCAGCTGAAGATCCCGTAGCAGCAGCACCACGCCCTCTTCCTGC
TGGAGGTAGACTTACGTTGCGACCGGCCCTCCGCCTTCCTTCACTGCTCCTCGTACATGTCTGCGCACGC
CCCGAGAAGCCACCAGGCCAGGTCACTCGACTCCGAGCCCTTCCGCTCACCCAAGGACAGCTCGTCCTTG
TCTGGTCTGACGAGCATGTCGGTAGCAAATGCCTTTGGACGTACGAGATACAGTTTAGCCAGGACGGTAA
GGCATATACTCCTGTTAGTAGGAAGCCATCTACTTTTAACCTTTTTGTATTCAGTCCCGACACGGGTGCT
GTCAGCGGGTCTTACCGGGTCCGCGCCCTGGATTATTGGGCCAGACCCGGACCTTTCTCCGATCCTGTCC
CCTATCTCGAGGTGCCCGTGCCAAGGGGGCCTCCCAGCCCCGGCAATCCATAATGAACCCAGCTTTCTTG
TACAAAGTGGTTGTACAAGTAGGAATTCGTCGAGGGACCTAATAACTTCGTATAGCATACATTATACGAA
GTTATACATGTTTAAGGGTTCCGGTTCCACTAGGTACAATTCGATATCAAGCTTATCGATAATCAACCTC
TGGATTACAAAATTTGTGAAAGATTGACTGGTATTCTTAACTATGTTGCTCCTTTTACGCTATGTGGATA
CGCTGCTTTAATGCCTTTGTATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACGTGGCGTGGTGTGCACTGTGT
TTGCTGACGCAACCCCCACTGGTTGGGGCATTGCCACCACCTGTCAGCTCCTTTCCGGGACTTTCGCTTT
CCCCCTCCCTATTGCCACGGCGGAACTCATCGCCGCCTGCCTTGCCCGCTGCTGGACAGGGGCTCGGCTG
TTGGGCACTGACAATTCCGTGGTGTTGTCGGGGAAATCATCGTCCTTTCCTTGGCTGCTCGCCTGTGTTG
CCACCTGGATTCTGCGCGGGACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCCCTCAGACGAGTCGGATCTCC
CTTTGGGCCGCCTCCCCGCATCGATACCGTCGACCTCGATCGAGACCTAGAAAAACATGGAGCAATCACA
AGTAGCAATACAGCAGCTACCAATGCTGATTGTGCCTGGCTAGAAGCACAAGAGGAGGAGGAGGTGGGTT
TTCCAGTCACACCTCAGGTACCTTTAAGACCAATGACTTACAAGGCAGCTGTAGATCTTAGCCACTTTTT
AAAAGAAAAGGGGGGACTGGAAGGGCTAATTCACTCCCAACGAAGACAAGATATCCTTGATCTGTGGATC
TACCACACACAAGGCTACTTCCCTGATTGGCAGAACTACACACCAGGGCCAGGGATCAGATATCCACTGA
CCTTTGGATGGTGCTACAAGCTAGTACCAGTTGAGCAAGAGAAGGTAGAAGAAGCCAATGAAGGAGAGAA
CACCCGCTTGTTACACCCTGTGAGCCTGCATGGGATGGATGACCCGGAGAGAGAAGTATTAGAGTGGAGG
TTTGACAGCCGCCTAGCATTTCATCACATGGCCCGAGAGCTGCATCCGGACTGTAC [symbol] GGTCTCTCTGGT
TAGACCAGATCTGAGCCTGGGAGCTCTCTGGCTAACTAGGGAACCCACTGCTTAAGCCTCAATAAAGCTT
GCCTTGAGTGCTTCAAGTAGTGTGTGCCCGTCTGTTGTGTGACTCTGGTAACTAGAGATCCCTCAGACCC
TTTTAGTCAGTGTGGAAAATCTCTAGCA

TABLE 5
Coding sequences for FAM1 B12 vector (SEQ ID NO: 39). WPRE, woodchuck
hepatitis virus posttranscriptional regulatory element (#); FEEK, B
cell specific promoter (†); T2A, 2A peptide from Thosea asigna virus
(italics); M1 (*) and M2 ( [symbol] ), transmembrane coding regions;
B12 heavy chain (Δ); B12 light chain (V).

TGTGTAATTGTTAATTTCTCTGTCCCACTCCATCCAGGTCGTGTGATTCCAAATCTGTTCCAGAGATTTA
TTACTCCAACTAGCATTCCAAGGCACAGCAGTGGTGCAAATGAGTTTTCCAGAGCAACCCCAAATCCCCA
GGAGCTGTTGATCCTTTAGGTATCTTTCCACAGCCAGGATTCTTGCCTGGAGCTGCTTGATGCCCCAGAC
TGTGAGTTGCAACAGATGCTGTTGCGCCTCAATAGCCCTCAGCAAATTGTTCTGCTGCTGCACTATACCA
GACAATAATTGTCTGGCCTGTACCGTCAGCGTCATTGACGCTGCGCCCATAGTGCTTCCTGCTGCTCCCA
AGAACCCAAGGAACAAAGCTCCTATTCCCACTGCTCTTTTTTCTCTCTGCACCACTCTTCTCTTTGCCTT
GGTGGGTGCTACTCCTAATGGTTCAATTTTTACTACTTTATATTTATATAATTCACTTCTCCAATTGTCC
CTCATATCTCCTCCTCCAGGTCTGAAGATCAGCGGCCGCTTGCTGTGCGGTGGTCTTACTTTTGTTTTGC
TCTTCCTCTATCTTGTCTAAAGCTTCCTTGGTGTCTTTTATCTCTATCCTTTGATGCACACAATAGAGGG
TTGCTACTGTATTATATAATGATCTAAGTTCTTCTGATNCCTGTCTGAAGGGATGGTTGTAGCTGTCCCA
GTATTTGTCTACAGCCTTCTGATGTTTCTAACAGGCCAGGATTAACTGCGAATCGTTCTAGCTCCCTGCT
TGCCCATACTATATGTTTTAATTTATATTTTTTCTTTCCCCCTGGCCTTAACCGAATTTTTTCCCATCGC
GATCTAATTCTCCCCCGCTTAATACTGACGCTCTCGCACCCATCTCTCTCCTTCTAGCCTCCGCTAGTCA
AAATTTTTGGCGTACTCACCAGTCGCCGCCCCTCGCCTCTTGCCGTGCGCGCTTCAGCAAGCCGAGTCCT
GCGTCGAGAGAGCTCCTCTGGTTTCCCTTTCGCTTTCAAGTCCCTGTTCGGGCGCCACTGCTAGAGATTT
TCCACACTGACTAAAAGGGTCTGAGGGATCTCTAGTTACCAGAGTCACACAACAGACGGGCACACACTAC
TTGAAGCACTCAAGGCAAGCTTTATTGAGGCTTAAGCAGTGGGTTCCCTAGTTAGCCAGAGAGCTCCCAG
GCTCAGATCTGGTCTAACCAGAGAGACCCAGTACAGGCAAAACGCGCTGCTTATATAGACCTCCCACCGT
ACACGCCTACCGCCCATTTGCGTCAATGGGGCGGAGTTGTTACGACATTTTGGAAAGTCCCGTTGATTTT
GGTGCCAAAACAAACTCCCATTGACGTCAATGGGGTGGAGACTTGGAAATCCCCGTGAGTCAAACCGCTA
TCCACGCCCATTGATGTACTGCCAAAACCGCATCACCATGGTAATAGCGATGACTAATACGTAGATGTAC
TGCCAAGTAGGAAAGTCCCATAAGGTCATGTACTGGGCATAATGCCAGGCGGGCCATTTACCGTCATTGA
CGTCAATAGGGGGCGTACTTGGCATATGATACACTTGATGTACTGCCAAGTGGGCAGTTTACCGTAAATA
CTCCACCCATTGACGTCAATGGAAAGTCCCTATTGGCGTTACTATGGGAACATACGTCATTATTGACGTC
AATGGGCGGGGGTCGTTGGGCGGTCAGCCAGGCGGGCCATTTACCGTAAGTTATGTAACGCGGAACTCCA
TATATGGGCTATGAACTAATGACCCCGTAATTGATTACTATTAATAACTAGTCAATAATCAATGTCAACG
CGTATATCTGGCCCGTACATCGCGAAGCAGCGCAAAACGCCTAACCCTAAGCAGATTCTTCATGCAATTG
TCGGTCAAGCCTTGCCTTGTTGTAGCTTAAATTTTGCTCGCGCACTACTCAGCGACCTCCAACACACAAG
CAGGGAGCAGATACTGGCTTAACTATGCGGCATCAGAGCAGATTGTACTGAGAGTGCACCATAGGGGATC
GGGAGATCTCCCGATCCGTCGACGTCAGGTGGCACTTTTCGGGGAAATGTGCGCGGAACCCCTATTTGTT
TATTTTTCTAAATACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCGGGCAAGAGCAA
CTCGGTCGCCGCATACACTATTCTCAGAATGACTTGGTTGAGTACTCACCAGTCACAGAAAAGCATCTTA
CGGATGGCATGACAGTAAGAGAATTATGCAGTGCTGCCATAACCATGAGTGATAACACTGCGGCCAACTT
ACTTCTGACAACGATCGGAGGACCGAAGGAGCTAACCGCTTTTTTGCACAACATGGGGGATCATGTAACT
CGCCTTGATCGTTGGGAACCGGAGCTGAATGAAGCCATACCAAACGACGAGCGTGACACCACGATGCCTG
TAGCAATGGCAACAACGTTGCGCAAACTATTAACTGGCGAACTACTTACTCTAGCTTCCCGGCAACAATT
AATAGACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGATGGTA
AGCCCTCCCGTATCGTAGTTATCTACACGACGGGGAGTCAGGCAACTATGGATGAACGAAATAGACAGAT
CGCTGAGATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTCATGACCA
AAATCCCTTAACGTGAGTTTTCGTTCCACTGAGCGTCAGACCCCGTAGAAAAGATCAAAGGATCTTCTTG
AGATCCTTTTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGATACCAAATA
CTGTTCTTCTAGTGTAGCCGTAGTTAGGCCACCACTTCAAGAACTCTGTAGCACCGCCTACATACCTCGC
TCTGCTAATCCTGTTACCAGTGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGAAGCGGAAGAGC
GCCCAATACGCAAACCGCCTCTCCCCGCGCGTTGGCCGATTCATTAATGCAGCTGGCACGACAGGTTTCC
CGACTGGAAAGCGGGCAGTGAGCGCAACGCAATTAATGTGAGTTAGCTCACTCATTAGGCACCCCAGGCT
TTACACTTTATGCTTCCGGCTCGTATGTTGTGTGGAATTGTGAGCGGATAACAATTTCACACAGGAAACA
GCTATGACCATGATTACGCCAAGCTCTAGCTAGAGGTCGACGGTATACAGACATGATAAGATACATTGAT
GAGTTTGGACAAACCACAACTAGAATGCAGTGAAAAAAATGCTTTATTTGTGAAATTTGTGATGCTATTG
CTTTATTTGTAACCATTATAAGCTGCAATAAACAAGTTGGGGTGGGCGAAGAACTCCAGCATGAGATCCC
CGCGCTGGAGGATCATCCAGCCGGCGTCCCGGAAAACGATTCCGAAGCCCAACCTTTCATAGAAGGCGGC
GGTGGAATCGAAATCTCGTAGCACGTGTCAGTCCTGCTCCTCGGCCACGAAGTGCACGCAGTTGCCGGCC
GGGTCGCGCAGGGCGAACTCCCGCCCCCACGGCTGCTCGCCGATCTCGGTCATGGCCGGCCCGGAGGCGT
CCCGGAAGTTCGTGGACACGACCTCCGACCACTCGGCGTACAGCTCGTCCAGGCCGCGCACCCACACCCA
GGCCAGGGTGTTGTCCGGCACCACCTGGTCCTGGACCGCGCTGATGAACAGGGTCACGTCGTCCCGGACC
ACACCGGCGAAGTCGTCCTCCACGAAGTCCCGGGAGAACCCGAGCCGGTCGGTCCAGAACTCGACCGCTC
CGGCGACGTCGCGCGCGGTGAGCACCGGAACGGCACTGGTCAACTTGGCCATGGTTTAGTTCCTCACCTT
GTCGTATTATACTATGCCGATATACTATGCCGATGATTAATTGTCAACACGTGCTGATCAGATCCGAAAA
TGGATATACAAGCTCCCGGGAGCTTTTTGCAAAAGCCTAGGCCTCCAAAAAAGCCTCCTCACTACTTCTG
GAATAGCTCAGAGGCAGAGGCGGCCTCGGCCTCTGCATAAATAAAAAAAATTAGTCAGCCATGGGGCGGA
GAATGGGCGGAACTGGGCGGAGTTAGGGGCGGGATGGGCGGAGTTAGGGGCGGGACTATGGTTGCTGACT
AATTGAGATGCATGCTTTGCATACTTCTGCCTGCTGGGGAGCCTGGGGACTTTCCACACCTGGTTGCTGA
CTAATTGAGATGCATGCTTTGCATACTTCTGCCTGCTGGGGAGCCTGGGGACTTTCCACACCCTAACTGA
CACACATTCCACAGAATTAATTCGCGTTAAATTTTTGTTAAATCAGCTCATTTTTTAACCAATAGGCCGA
AATCGGCAAAATCCCTTATAAATCAAAAGAATAGACCGAGATAGGGTTGAGTGTTGTTCCAGTTTGGAAC
AAGAGTCCACTATTAAAGAACGTGGACTCCAACGTCAAAGGGCGAAAAACCGTCTATCAGGGCGATGGCC
CACTACGTGAACCATCACCCTAATCAAGTTTTTTGGGGTCGAGGTGCCGTAAAGCACTAAATCGGAACCC
TAAAGGGAGCCCCCGATTTAGAGCTTGACGGGGAAAGCCGGCGAACGTGGCGAGAAAGGAAGGGAAGAAA
GCGAAAGGAGCGGGCGCTAGGGCGCTGGCAAGTGTAGCGGTCACGCTGCGCGTAACCACCACACCCGCCG
CGCTTAATGCGCCGCTACAGGGCGCGTGGGGATACCCCCTAGAGCCCCAGCTGGTTCTTTCCGCCTCAGA
AGCCATAGAGCCCACCGCATCCCCAGCATGCCTGCTATTGTCTTCCCAATCCTCCCCCTTGCTGTCCTGC
CCCACCCCACCCCCCAGAATAGAATGACACCTACTCAGACAATGCGATGCAATTTCCTCATTTTATTAGG
AAAGGACAGTGGGAGTGGCACCTTCCAGGGTCAAGGAAGGCACGGGGGAGGGGCAAACAACAGATGGCTG
GCAACTAGAAGGCACAGTCGAGGCTGATCAGCGGGTTTAAACGGGCCCTGCTAGAGATTTTCCACACTGA
CTAAAAGGGTCTGAGGGATCTCTAGTTACCAGAGTCACACAACAGACGGGCACACACTACTTGAAGCACT
CAAGGCAAGCTTTATTGAGGCTTAAGCAGTGGGTTCCCTAGTTAGCCAGAGAGCTCCCAGGCTCAGATCT
GGTCTAACCAGAGAGACCCAGTACAGTCCGGATGCAGCTCTCGGGCCATGTGATGAAATGCTAGGCGGCT
GTCAAACCTCCACTCTAATACTTCTCTCTCCGGGTCATCCATCCCATGCAGGCTCACAGGGTGTAACAAG
CGGGTGTTCTCTCCTTCATTGGCTTCTTCTACCTTCTCTTGCTCAACTGGTACTAGCTTGTAGCACCATC
CAAAGGTCAGTGGATATCTGATCCCTGGCCCTGGTGTGTAGTTCTGCCAATCAGGGAAGTAGCCTTGTGT
GTGGTAGATCCACAGATCAAGGATATCTTGTCTTCGTTGGGAGTGAATTAGCCCTTCCAGTCCCCCCTTT
TCTTTTAAAAAGTGGCTAAGATCTACAGCTGCCTTGTAAGTCATTGGTCTTAAAGGTACCTGAGGTGTGA
CTGGAAAACCCACCTCCTCCTCCTCTTGTGCTTCTAGCCAGGCACAATCAGCATTGGTAGCTGCTGTATT
GCTACTTGTGATTGCTCCATGTTTTTCTAGGTCTCGATCGAGGTCGACGGTA [symbol] CGATGCGGGGAGGCGGC
CCAAAGGGAGATCCGACTCGTCTGAGGGCGAAGGCGAAGACGCGGAAGAGGCCGCAGAGCCGGCAGCAGG
CCGCGGGAAGGAAGGTCCGCTGGATTGAGGGCCGAAGGGACGTAGCAGAAGGACGTCCCGCGCAGAATGC
AGGTGGCAACACAGGCGAGCAGCCAAGGAAAGGACGATGATTTCCCCGACAACACCACGGAATTGTCAGT
GCCCAACAGCCGAGCCCCTGTCCAGCAGCGGGCAAGGCAGGCGGCGATGAGTTCCGCCGTGGCAATAGGG
AGGGGGAAAGCGAAAGTCCCGGAAAGGAGCTGACAGGTGGTGGCAATGCCCCAACCAGTGGGGGTTGCGT
CAGCAAACACAGTGCACACCACGCCACGTTGCCTGACAACGGGCCACAACTCCTCATAAAGAGACAGCAA
CCAGGATTTATACAAGGAGGAGAAAATGAAAGCCATACGGGAAGCAATAGCATGATACAAAGGCATTAAA
GCAGCGTATCCACATAGCGTAAAAGGAGCAACATAGTTAAGAATACCAGTCAATCTTTCACAAATTTTGT
AATCCAGAGGTTGATTATCGATAAGCTTGATATCGAATTGGGAGGGACTAGAGTCGCGGCCGCTTTACTT
GTACAGTTTAACATCTCGAGCCATAAAGATGGTTAATTAACCCACCCAAGATC [symbol] AAACCGGTGAGTTTCA
TGGTTACTTGCCTGAGAAGATTAAAAAAAGTAATGCTACCTTATGAGGGAGAGTCCCAGGGACCAAGATA
GCAACTGTCATAGCAACCGTCACACTGCTTTGGTCAAGGAGAAGACCCTTTGGGGAACTGAAAACAGAAC
CTTGAGCACATCTGTTGCTTTCGCTCCCATCCTCCTCCAACAGGGCTGGGTGGAGCACTCCACACCCTTT
CACCGGTCGTACGGCTCAGCCAGAGTAAAAATCACACCCATGACCTGGCCACTGAGGGCTTGATCAATTC
ACTTTGAATTTGGCATTAAATACCATTAAGGTATATTAACTGATTTTAAAATAAGATATATTCGTGACCA
TGTTTTTAACTTTCAAAAATGTAGCTGCCAGTGTGTGATTTTATTTCAGTTGTACAAAATATCTAAACCT
ATAGCAATGTGATTAATAAAAACTTAAACATATTTTCCAGTACCTTAATTCTGTGATAGGAAAATTTTAA
TCTGAGTATTTTAATTTCATAATCTCTAAAATAGTTTAATGATTTGTCATTGTGTTGCTGTCGTTTACCC
CAGCTGATCTCAAAAGTGATATTTAAGGAGATTATTTTGGTCTGCAACAACTTGATAGGGCTCAGCCTCT
CCCACCCAACGGGTGGAATCCCCCAGAGGGGGATTTCCAAGAGGCCACCTGGCAGTTGCTGAGGGTCAGA
AGTGAAGGTAGCCACTTCCTCTTAGGCAGGTGGCCAAGATTACAGTTGACCCGTACGTGCAGCTGTGCCC
AGCCTGCCCCATCCCCTGCTCATTTGCATGTTCCCAGAGCACAACCTCCTGCCCTGAAGCCTTATTAATA
GGCTGGTCACACTTTGTGCAGGAGTCAGACTCAGTCAGGACACAGCTACGAAGCTTGGTACCGAGCTCGG
ATCTCCACCCCGTAGCCACC [symbol] GGGTGTGCCCACTCAGGTCCTGGGGTTGCTGCTGCTGTGGCTTACAGA
TGCCAGATGTGAGATCGAGGAAATTGTGTTGACTCAGAGCCCCGGAACTCTGTCCCTCAGTCCCGGCGAA
AGGGCAACATTCAGTTGTAGGTCTTCCCATTCAATTCGAAGTAGGCGCGTTGCGTGGTACCAACACAAAC
CCGGCCAAGCTCCACGCCTGGTGATCCACGGGGTGTCAAATAGGGCATCTGGGATCTCCGATAGGTTTTC
AGGGTCAGGATCAGGTACGGATTTCACCCTTACCATCACACGGGTTGAACCCGAGGATTTCGCCCTTTAT
TACTGCCAGGTATATGGGGCGAGCAGTTATACGTTTGGTCAGGGGACCAAGCTCGAGCGAAAACGGACCG
TCGCGGCCCCAAGCGTCTTTATCTTCCCACCCTCTGACGAACAGCTCAAATCCGGTACAGCTTCTGTAGT
TTGCTTGCTCAACAACTTTTACCCACGGGAGGCGAAAGTTCAATGGAAAGTGGATAATGCCCTTCAATCC
GGGAACAGCCAAGAAAGTGTGACGGAACAGGACAGCAAAGACTCTACCTATTCACTCAGTAGCACTCTGA
CTTTGTCCAAGGCCGATTATGAAAAACATAAGGTTTACGCGTGCGAGGTTACACACCAAGGTCTGCGAAG
CCCAGTAACAAAATCCTTCAATAGGGGTGAGTGCGAGGGCAGAGGAAGTCTGTTAACATGCGGTGACGTC
GAGGAGAATCCTGGCCCA [symbol] AGTCCAACTGGTACAATCCGGAGCTGAAGTGAAAAAACCAGGGGCATCCG
TGAAAGTCTCATGCCAAGCCTCCGGTTACCGATTTAGTAACTTTGTGATACACTGGGTTCGGCAAGCCCC
AGGACAAAGATTCGAGTGGATGGGCTGGATAAACCCATATAATGGTAACAAAGAATTCTCAGCTAAATTC
CAAGACCGGGTAACATTTACGGCGGATACTTCAGCTAACACTGCATACATGGAACTTCGCAGCTTGAGGT
CCGCGGACACAGCCGTATATTACTGCGCGAGGGTTGGCCCATATAGCTGGGACGACAGTCCGCAGGACAA
TTATTACATGGACGTGTGGGGGAAGGGTACGACCGTAATAGTCAGTAGTGCTAGCACCAAGGGCCCATCG
GTCTTCCCCCTGGCACCCTCCTCCAAGAGCACCTCTGGGGGCACAGCGGCCCTGGGCTGCCTGGTCAAGG
ACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAAAGTTGAGCCCA
AATCTTGTGACAAAACTCACACATGCCCACCGTGCCCAGCACCTGAACTCCTGGGGGGACCGTCAGTCTT
CCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTATGTTGACGGCGTGGAGGTGCATAATGCCA
AGACAAAGCCGCGGGAGGAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGC
TGACCAAGAATCAAGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTC
TTCCTCTACTCAAAACTCACCGTGGACAAGAGCAGGTGGCAGCAGGGGAACGTCTTCTCATGCTCCGTGA
TGCATGAGGCTCTGCACAACCACTACACACAGAAGAGCCTCTCCCTGTCTCCGGGTAAATGAGTGCCACG
GCCGGCAAGCCCCCGCTCCCCAGGCTCTCGGGGTCGCGCGAGGATGCTTGGCACGTACCCCGTGTACATA
CTTCCCAGGCACCCAGCATGGAAATAAAGCACCCAGCGCTTCCCTGGGCCCCTGCGAGACTGTGATGGTT
CTTTCCACGGGTCAGGCCGAGTCTGAGGCCTGAGTGGCATGAGGGAGGCAGAGTGGGTCCCACTGTCCCC
ACACTGGCCCAGGCTGTGGTGGGGAGCTGACCTCAGGACATTGTTGGCCCATCCCGGCCGGGCCCTACAT
CCTGGGTCCTGCCACAGAGGGAATCACCCCCAGAGGCCCAAGCCCAGGGGGACACAGCACTGACCACCCC
CTTCCTGTCCAGAG [symbol] TGCAACTGGAGGAGAGCTGTGCGGAGGCGCAGGACGGGGAGCTGGACGGGCTGTG
GACGACCATCACCATCTTCATCACACTCTTCCTGTTAAGCGTGTGCTACAGTGCCACCGTCACCTTCTTC
AAG [symbol] GAAGTGGATCTTCTCCTCGGTGGTGGACCTGAAGCAGACCATCATCCCCGACTACAGGAACATGA
TCGGACAGGGGGCCTAGGGCCACCCTCTGCGGGGTGTCCAGGGCGCGCCAATAAAATATCTTTATTTTCA
TTACATCTGTGTGTTGGTTTTTTGTGTGAATCGATAGTACTAACATACGCTCTCCATCAAAACAAAACGA
AACAAAACAAACTAGCAAAATAGGCTGTCCCCAGTGCAAGTGCAGGTGCCAGAACATTTCTCTATTTAAA
TGCTAGAATTAACCAAACTGGATCTCTGCTGTCCCTGTAATAAACCCGAAAATTTTGAATTTTTGTAATT
TGTTTTTGTAATTCTTTAGTTTGTATGTCTGTTGCTATTATGTCTACTATTCTTTCCCCTGCACTGTACC
CCCCAATCCCCCCTTTTCTTTTAAAATTGTGGATGAATACTGCCATTTGTCTGCAGAATTGGCGCACGCA
GTGCCGATCCGTTCACTAATCGAATGGATCTGTCTCTGTCTCTCTCTCCACCTTCTTCTTCTATTCCTTC
GGGCCTGTCGGGTCCCCTCGGGGTTGGGAGGTGGGTCTGAAACGATAATGGTGAATATCCCTGCCTAACT
CTATTCACTATAGAAAGTACAGCAAAAACTATTCTTAAACCTACCAAGCCTCCTACTATCATTATGAATA
ATTTTATATACCACAGCCAATTTGTTATGTTAAACCAATTCCACAAACTTGCCCATTTATCTAATTCCAA
TAATTCTTGTTCATTCTTTTCTTGCTGGTTTTGCGATTCTTCAATTAAGGAGTGTATTAAGCT

TABLE 6
Coding sequences for FAM1 anti PE vector (SEQ ID NO: 40). WPRE, woodchuck
hepatitis virus posttranscriptional regulatory element (#); FEEL B cell
specific promoter (†): T2A, 2A peptide from Thosea asigna virus (italics):
MI (*) and M2 ( [symbol] ), transmembrane coding regions; Anti-PE Light
Chain (Δ); Anti-PE Heavy Chain (V).

TGTGTAATTGTTAATTTCTCTGTCCCACTCCATCCAGGTCGTGTGATTCCAAATCTGTTCCAGAGATTTA
TTACTCCAACTAGCATTCCAAGGCACAGCAGTGGTGCAAATGAGTTTTCCAGAGCAACCCCAAATCCCCA
GGAGCTGTTGATCCTTTAGGTATCTTTCCACAGCCAGGATTCTTGCCTGGAGCTGCTTGATGCCCCAGAC
TGTGAGTTGCAACAGATGCTGTTGCGCCTCAATAGCCCTCAGCAAATTGTTCTGCTGCTGCACTATACCA
GACAATAATTGTCTGGCCTGTACCGTCAGCGTCATTGACGCTGCGCCCATAGTGCTTCCTGCTGCTCCCA
AGAACCCAAGGAACAAAGCTCCTATTCCCACTGCTCTTTTTTCTCTCTGCACCACTCTTCTCTTTGCCTT
GGTGGGTGCTACTCCTAATGGTTCAATTTTTACTACTTTATATTTATATAATTCACTTCTCCAATTGTCC
CTCATATCTCCTCCTCCAGGTCTGAAGATCAGCGGCCGCTTGCTGTGCGGTGGTCTTACTTTTGTTTTGC
TCTTCCTCTATCTTGTCTAAAGCTTCCTTGGTGTCTTTTATCTCTATCCTTTGATGCACACAATAGAGGG
TTGCTACTGTATTATATAATGATCTAAGTTCTTCTGATNCCTGTCTGAAGGGATGGTTGTAGCTGTCCCA
GTATTTGTCTACAGCCTTCTGATGTTTCTAACAGGCCAGGATTAACTGCGAATCGTTCTAGCTCCCTGCT
TGCCCATACTATATGTTTTAATTTATATTTTTTCTTTCCCCCTGGCCTTAACCGAATTTTTTCCCATCGC
GATCTAATTCTCCCCCGCTTAATACTGACGCTCTCGCACCCATCTCTCTCCTTCTAGCCTCCGCTAGTCA
AAATTTTTGGCGTACTCACCAGTCGCCGCCCCTCGCCTCTTGCCGTGCGCGCTTCAGCAAGCCGAGTCCT
GCGTCGAGAGAGCTCCTCTGGTTTCCCTTTCGCTTTCAAGTCCCTGTTCGGGCGCCACTGCTAGAGATTT
TCCACACTGACTAAAAGGGTCTGAGGGATCTCTAGTTACCAGAGTCACACAACAGACGGGCACACACTAC
TTGAAGCACTCAAGGCAAGCTTTATTGAGGCTTAAGCAGTGGGTTCCCTAGTTAGCCAGAGAGCTCCCAG
GCTCAGATCTGGTCTAACCAGAGAGACCCAGTACAGGCAAAACGCGCTGCTTATATAGACCTCCCACCGT
ACACGCCTACCGCCCATTTGCGTCAATGGGGCGGAGTTGTTACGACATTTTGGAAAGTCCCGTTGATTTT
GGTGCCAAAACAAACTCCCATTGACGTCAATGGGGTGGAGACTTGGAAATCCCCGTGAGTCAAACCGCTA
TCCACGCCCATTGATGTACTGCCAAAACCGCATCACCATGGTAATAGCGATGACTAATACGTAGATGTAC
TGCCAAGTAGGAAAGTCCCATAAGGTCATGTACTGGGCATAATGCCAGGCGGGCCATTTACCGTCATTGA
CGTCAATAGGGGGCGTACTTGGCATATGATACACTTGATGTACTGCCAAGTGGGCAGTTTACCGTAAATA
CTCCACCCATTGACGTCAATGGAAAGTCCCTATTGGCGTTACTATGGGAACATACGTCATTATTGACGTC
AATGGGCGGGGGTCGTTGGGCGGTCAGCCAGGCGGGCCATTTACCGTAAGTTATGTAACGCGGAACTCCA
TATATGGGCTATGAACTAATGACCCCGTAATTGATTACTATTAATAACTAGTCAATAATCAATGTCAACG
CGTATATCTGGCCCGTACATCGCGAAGCAGCGCAAAACGCCTAACCCTAAGCAGATTCTTCATGCAATTG
TCGGTCAAGCCTTGCCTTGTTGTAGCTTAAATTTTGCTCGCGCACTACTCAGCGACCTCCAACACACAAG
CAGGGAGCAGATACTGGCTTAACTATGCGGCATCAGAGCAGATTGTACTGAGAGTGCACCATAGGGGATC
GGGAGATCTCCCGATCCGTCGACGTCAGGTGGCACTTTTCGGGGAAATGTGCGCGGAACCCCTATTTGTT
TATTTTTCTAAATACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCGGGCAAGAGCAA
CTCGGTCGCCGCATACACTATTCTCAGAATGACTTGGTTGAGTACTCACCAGTCACAGAAAAGCATCTTA
CGGATGGCATGACAGTAAGAGAATTATGCAGTGCTGCCATAACCATGAGTGATAACACTGCGGCCAACTT
ACTTCTGACAACGATCGGAGGACCGAAGGAGCTAACCGCTTTTTTGCACAACATGGGGGATCATGTAACT
CGCCTTGATCGTTGGGAACCGGAGCTGAATGAAGCCATACCAAACGACGAGCGTGACACCACGATGCCTG
TAGCAATGGCAACAACGTTGCGCAAACTATTAACTGGCGAACTACTTACTCTAGCTTCCCGGCAACAATT
AATAGACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGATGGTA
AGCCCTCCCGTATCGTAGTTATCTACACGACGGGGAGTCAGGCAACTATGGATGAACGAAATAGACAGAT
CGCTGAGATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTCATGACCA
AAATCCCTTAACGTGAGTTTTCGTTCCACTGAGCGTCAGACCCCGTAGAAAAGATCAAAGGATCTTCTTG
AGATCCTTTTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGATACCAAATA
CTGTTCTTCTAGTGTAGCCGTAGTTAGGCCACCACTTCAAGAACTCTGTAGCACCGCCTACATACCTCGC
TCTGCTAATCCTGTTACCAGTGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGAAGCGGAAGAGC
GCCCAATACGCAAACCGCCTCTCCCCGCGCGTTGGCCGATTCATTAATGCAGCTGGCACGACAGGTTTCC
CGACTGGAAAGCGGGCAGTGAGCGCAACGCAATTAATGTGAGTTAGCTCACTCATTAGGCACCCCAGGCT
TTACACTTTATGCTTCCGGCTCGTATGTTGTGTGGAATTGTGAGCGGATAACAATTTCACACAGGAAACA
GCTATGACCATGATTACGCCAAGCTCTAGCTAGAGGTCGACGGTATACAGACATGATAAGATACATTGAT
GAGTTTGGACAAACCACAACTAGAATGCAGTGAAAAAAATGCTTTATTTGTGAAATTTGTGATGCTATTG
CTTTATTTGTAACCATTATAAGCTGCAATAAACAAGTTGGGGTGGGCGAAGAACTCCAGCATGAGATCCC
CGCGCTGGAGGATCATCCAGCCGGCGTCCCGGAAAACGATTCCGAAGCCCAACCTTTCATAGAAGGCGGC
GGTGGAATCGAAATCTCGTAGCACGTGTCAGTCCTGCTCCTCGGCCACGAAGTGCACGCAGTTGCCGGCC
GGGTCGCGCAGGGCGAACTCCCGCCCCCACGGCTGCTCGCCGATCTCGGTCATGGCCGGCCCGGAGGCGT
CCCGGAAGTTCGTGGACACGACCTCCGACCACTCGGCGTACAGCTCGTCCAGGCCGCGCACCCACACCCA
GGCCAGGGTGTTGTCCGGCACCACCTGGTCCTGGACCGCGCTGATGAACAGGGTCACGTCGTCCCGGACC
ACACCGGCGAAGTCGTCCTCCACGAAGTCCCGGGAGAACCCGAGCCGGTCGGTCCAGAACTCGACCGCTC
CGGCGACGTCGCGCGCGGTGAGCACCGGAACGGCACTGGTCAACTTGGCCATGGTTTAGTTCCTCACCTT
GTCGTATTATACTATGCCGATATACTATGCCGATGATTAATTGTCAACACGTGCTGATCAGATCCGAAAA
TGGATATACAAGCTCCCGGGAGCTTTTTGCAAAAGCCTAGGCCTCCAAAAAAGCCTCCTCACTACTTCTG
GAATAGCTCAGAGGCAGAGGCGGCCTCGGCCTCTGCATAAATAAAAAAAATTAGTCAGCCATGGGGCGGA
GAATGGGCGGAACTGGGCGGAGTTAGGGGCGGGATGGGCGGAGTTAGGGGCGGGACTATGGTTGCTGACT
AATTGAGATGCATGCTTTGCATACTTCTGCCTGCTGGGGAGCCTGGGGACTTTCCACACCTGGTTGCTGA
CTAATTGAGATGCATGCTTTGCATACTTCTGCCTGCTGGGGAGCCTGGGGACTTTCCACACCCTAACTGA
CACACATTCCACAGAATTAATTCGCGTTAAATTTTTGTTAAATCAGCTCATTTTTTAACCAATAGGCCGA
AATCGGCAAAATCCCTTATAAATCAAAAGAATAGACCGAGATAGGGTTGAGTGTTGTTCCAGTTTGGAAC
AAGAGTCCACTATTAAAGAACGTGGACTCCAACGTCAAAGGGCGAAAAACCGTCTATCAGGGCGATGGCC
CACTACGTGAACCATCACCCTAATCAAGTTTTTTGGGGTCGAGGTGCCGTAAAGCACTAAATCGGAACCC
TAAAGGGAGCCCCCGATTTAGAGCTTGACGGGGAAAGCCGGCGAACGTGGCGAGAAAGGAAGGGAAGAAA
GCGAAAGGAGCGGGCGCTAGGGCGCTGGCAAGTGTAGCGGTCACGCTGCGCGTAACCACCACACCCGCCG
CGCTTAATGCGCCGCTACAGGGCGCGTGGGGATACCCCCTAGAGCCCCAGCTGGTTCTTTCCGCCTCAGA
AGCCATAGAGCCCACCGCATCCCCAGCATGCCTGCTATTGTCTTCCCAATCCTCCCCCTTGCTGTCCTGC
CCCACCCCACCCCCCAGAATAGAATGACACCTACTCAGACAATGCGATGCAATTTCCTCATTTTATTAGG
AAAGGACAGTGGGAGTGGCACCTTCCAGGGTCAAGGAAGGCACGGGGGAGGGGCAAACAACAGATGGCTG
GCAACTAGAAGGCACAGTCGAGGCTGATCAGCGGGTTTAAACGGGCCCTGCTAGAGATTTTCCACACTGA
CTAAAAGGGTCTGAGGGATCTCTAGTTACCAGAGTCACACAACAGACGGGCACACACTACTTGAAGCACT
CAAGGCAAGCTTTATTGAGGCTTAAGCAGTGGGTTCCCTAGTTAGCCAGAGAGCTCCCAGGCTCAGATCT
GGTCTAACCAGAGAGACCCAGTACAGTCCGGATGCAGCTCTCGGGCCATGTGATGAAATGCTAGGCGGCT
GTCAAACCTCCACTCTAATACTTCTCTCTCCGGGTCATCCATCCCATGCAGGCTCACAGGGTGTAACAAG
CGGGTGTTCTCTCCTTCATTGGCTTCTTCTACCTTCTCTTGCTCAACTGGTACTAGCTTGTAGCACCATC
CAAAGGTCAGTGGATATCTGATCCCTGGCCCTGGTGTGTAGTTCTGCCAATCAGGGAAGTAGCCTTGTGT
GTGGTAGATCCACAGATCAAGGATATCTTGTCTTCGTTGGGAGTGAATTAGCCCTTCCAGTCCCCCCTTT
TCTTTTAAAAAGTGGCTAAGATCTACAGCTGCCTTGTAAGTCATTGGTCTTAAAGGTACCTGAGGTGTGA
CTGGAAAACCCACCTCCTCCTCCTCTTGTGCTTCTAGCCAGGCACAATCAGCATTGGTAGCTGCTGTATT
GCTACTTGTGATTGCTCCATGTTTTTCTAGGTCTCGATCGAGGTCGACGGTA [symbol] CGATGCGGGGAGGCGGC
CCAAAGGGAGATCCGACTCGTCTGAGGGCGAAGGCGAAGACGCGGAAGAGGCCGCAGAGCCGGCAGCAGG
CCGCGGGAAGGAAGGTCCGCTGGATTGAGGGCCGAAGGGACGTAGCAGAAGGACGTCCCGCGCAGAATGC
AGGTGGCAACACAGGCGAGCAGCCAAGGAAAGGACGATGATTTCCCCGACAACACCACGGAATTGTCAGT
GCCCAACAGCCGAGCCCCTGTCCAGCAGCGGGCAAGGCAGGCGGCGATGAGTTCCGCCGTGGCAATAGGG
AGGGGGAAAGCGAAAGTCCCGGAAAGGAGCTGACAGGTGGTGGCAATGCCCCAACCAGTGGGGGTTGCGT
CAGCAAACACAGTGCACACCACGCCACGTTGCCTGACAACGGGCCACAACTCCTCATAAAGAGACAGCAA
CCAGGATTTATACAAGGAGGAGAAAATGAAAGCCATACGGGAAGCAATAGCATGATACAAAGGCATTAAA
GCAGCGTATCCACATAGCGTAAAAGGAGCAACATAGTTAAGAATACCAGTCAATCTTTCACAAATTTTGT
AATCCAGAGGTTGATTATCGATAAGCTTGATATCGAATTGGGAGGGACTAGAGTCGCGGCCGCTTTACTT
GTACAGTTTAACATCTCGAGCCATAAAGATGGTTAATTAACCCACCCAAGATC [symbol] AAACCGGTGAGTTTCA
TGGTTACTTGCCTGAGAAGATTAAAAAAAGTAATGCTACCTTATGAGGGAGAGTCCCAGGGACCAAGATA
GCAACTGTCATAGCAACCGTCACACTGCTTTGGTCAAGGAGAAGACCCTTTGGGGAACTGAAAACAGAAC
CTTGAGCACATCTGTTGCTTTCGCTCCCATCCTCCTCCAACAGGGCTGGGTGGAGCACTCCACACCCTTT
CACCGGTCGTACGGCTCAGCCAGAGTAAAAATCACACCCATGACCTGGCCACTGAGGGCTTGATCAATTC
ACTTTGAATTTGGCATTAAATACCATTAAGGTATATTAACTGATTTTAAAATAAGATATATTCGTGACCA
TGTTTTTAACTTTCAAAAATGTAGCTGCCAGTGTGTGATTTTATTTCAGTTGTACAAAATATCTAAACCT
ATAGCAATGTGATTAATAAAAACTTAAACATATTTTCCAGTACCTTAATTCTGTGATAGGAAAATTTTAA
TCTGAGTATTTTAATTTCATAATCTCTAAAATAGTTTAATGATTTGTCATTGTGTTGCTGTCGTTTACCC
CAGCTGATCTCAAAAGTGATATTTAAGGAGATTATTTTGGTCTGCAACAACTTGATAGGGCTCAGCCTCT
CCCACCCAACGGGTGGAATCCCCCAGAGGGGGATTTCCAAGAGGCCACCTGGCAGTTGCTGAGGGTCAGA
AGTGAAGGTAGCCACTTCCTCTTAGGCAGGTGGCCAAGATTACAGTTGACCCGTACGTGCAGCTGTGCCC
AGCCTGCCCCATCCCCTGCTCATTTGCATGTTCCCAGAGCACAACCTCCTGCCCTGAAGCCTTATTAATA
GGCTGGTCACACTTTGTGCAGGAGTCAGACTCAGTCAGGACACAGCTACGAAGCTTGGTACCGAGCTCGG
ATCTCCACCCCGTAGCCACC [symbol] GGGTGTGCCCACTCAGGTCCTGGGGTTGCTGCTGCTGTGGCTTACAGA
TGCCAGATGTGAGATCGAGGACATTGTGTTGACCCAGACACCGGCTATTATGTCTGCCTCACTCGGCGAA
AGAGTAACGATGACTTGCACCGCGAGTTCTAGCGTCAGCTCTAGCTACCTTCATTGGTTTCAGCAAAAAC
CCGGGTCAAGTCCAAAGCTCTGGATCTACTCTACATCAAATCTGGCATCCGGTGTTCCTGGTAGGTTCTC
CGGCTCCGGCAGCGAAACTTCTTATTCCCTGATTATTGGCTCCCTCGAGACAGAAGATGCCGCAACCTAC
TACTGTCACCAATATCACCGGAGCCCGCCCACATTTGGAGGAGGAACTAAATTGGAAATCAAGACAGTTG
CAGCGCCTAGCGTATTTATTTTCCCACCCTCAGATGAGCAACTGAAATCCGGGACCGCATCTGTTGTCTG
TCTGCTGAACAACTTCTATCCCCGAGAGGCCAAAGTTCAGTGGAAAGTGGACAATGCGCTTCAATCTGGT
AACTCTCAAGAATCCGTCACTGAGCAGGATTCTAAGGATTCTACATATAGTCTTTCATCAACCCTTACAC
TTAGTAAGGCGGACTATGAAAAACACAAAGTGTACGCATGTGAGGTAACTCACCAGGGGCTGCGCTCCCC
GGTTACCAAAAGTTTTAATCGAGGGGAGTGCGAGGGCAGAGGAAGTCTGTTAACATGCGGTGACGTCGAG
GAGAATCCTGGCCCA [symbol] AAGTACAGCTGGAACAGAGTGGTGCAGAGTTGGCAAGACCAGGGGCCAGCGTGA
AACTCTCTTGCAAGGCGAGTGGATATACCTTTACAAGTTACGGTATTTCTTGGGTAAAACAGAGGACGGG
GCAGGGTCTGGAATGGATAGGTGAAATTTACCCTAAATCCGGCAATACATACTATAATGAAAAATTCAAA
GGACGAGCGACACTTACCGCTGATAAGTCTAGTTCCACTGCGTATATGGAACTCCGAAGCCTTACCAGCG
AGGACTCCACAGTGTATTTTTGCGCGCGACAGGGGTACTATGCAAATTCACAATTCACGTACTGGGGTCA
AGGGACACTTGTAACAGTAAGCGCAGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTGACGG
TGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAAAGTTGAGCCCAAATCTTGTGACAAAACTCACACAT
GCCCACCGTGCCCAGCACCTGAACTCCTGGGGGGACCGTCAGTCTTCCTCTTCCCCCCAAAACCCAAGGA
CACCCTCATGATCTCCCGGACCCCTGAGGTCACATGCGTGGTGGTGGACGTGAGCCACGAAGACCCTGAG
GTCAAGTTCAACTGGTATGTTGACGGCGTGGAGGTGCATAATGCCAAGACAAAGCCGCGGGAGGAGCAGT
ACAACAGCACGTACCGTGTGGTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAATCAAGTCAGCCTGA
CCTGCCTGGTCAAAGGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACTCAAAACTCACCGTG
GACAAGAGCAGGTGGCAGCAGGGGAACGTCTTCTCATGCTCCGTGATGCATGAGGCTCTGCACAACCACT
ACACACAGAAGAGCCTCTCCCTGTCTCCGGGTAAATGAGTGCCACGGCCGGCAAGCCCCCGCTCCCCAGG
CTCTCGGGGTCGCGCGAGGATGCTTGGCACGTACCCCGTGTACATACTTCCCAGGCACCCAGCATGGAAA
TAAAGCACCCAGCGCTTCCCTGGGCCCCTGCGAGACTGTGATGGTTCTTTCCACGGGTCAGGCCGAGTCT
GAGGCCTGAGTGGCATGAGGGAGGCAGAGTGGGTCCCACTGTCCCCACACTGGCCCAGGCTGTGGTGGGG
AGCTGACCTCAGGACATTGTTGGCCCATCCCGGCCGGGCCCTACATCCTGGGTCCTGCCACAGAGGGAAT
CACCCCCAGAGGCCCAAGCCCAGGGGGACACAGCACTGACCACCCCCTTCCTGTCCAGAG [symbol] TGCAACTGG
AGGAGAGCTGTGCGGAGGCGCAGGACGGGGAGCTGGACGGGCTGTGGACGACCATCACCATCTTCATCAC
ACTCTTCCTGTTAAGCGTGTGCTACAGTGCCACCGTCACCTTCTTCAAGGTGAA [symbol] GGATCTTCTCCTCG
GTGGTGGACCTGAAGCAGACCATCATCCCCGACTACAGGAACATGATCGGACAGGGGGCCTAGGGCCACC
CTCTGCGGGGTGTCCAGGGCGCGCCAATAAAATATCTTTATTTTCATTACATCTGTGTGTTGGTTTTTTG
TGTGAATCGATAGTACTAACATACGCTCTCCATCAAAACAAAACGAAACAAAACAAACTAGCAAAATAGG
CTGTCCCCAGTGCAAGTGCAGGTGCCAGAACATTTCTCTATTTAAATGCTAGAATTAACCAAACTGGATC
TCTGCTGTCCCTGTAATAAACCCGAAAATTTTGAATTTTTGTAATTTGTTTTTGTAATTCTTTAGTTTGT
ATGTCTGTTGCTATTATGTCTACTATTCTTTCCCCTGCACTGTACCCCCCAATCCCCCCTTTTCTTTTAA
AATTGTGGATGAATACTGCCATTTGTCTGCAGAATTGGCGCACGCAGTGCCGATCCGTTCACTAATCGAA
TGGATCTGTCTCTGTCTCTCTCTCCACCTTCTTCTTCTATTCCTTCGGGCCTGTCGGGTCCCCTCGGGGT
TGGGAGGTGGGTCTGAAACGATAATGGTGAATATCCCTGCCTAACTCTATTCACTATAGAAAGTACAGCA
AAAACTATTCTTAAACCTACCAAGCCTCCTACTATCATTATGAATAATTTTATATACCACAGCCAATTTG
TTATGTTAAACCAATTCCACAAACTTGCCCATTTATCTAATTCCAATAATTCTTGTTCATTCTTTTCTTG
CTGGTTTTGCGATTCTTCAATTAAGGAGTGTATTAAGCT

TABLE 7
Coding sequences for FAM2 B12 vector (SEQ ID NO: 41). WPRE, woodchuck
hepatitis virus posttranscriptional regulatory element (#); FEEK, B cell
specific promoter (†); T2A, 2A peptide from Thosea asigna virus (italics),
M1 (*) and M2 ( [symbol] ), transmembrane coding regions; B12 Light Chain (Δ); B12
Heavy Chain (V); intron (lower case).

TGTGTAATTGTTAATTTCTCTGTCCCACTCCATCCAGGTCGTGTGATTCCAAATCTGTTCCAGAGATTTA
TTACTCCAACTAGCATTCCAAGGCACAGCAGTGGTGCAAATGAGTTTTCCAGAGCAACCCCAAATCCCCA
GGAGCTGTTGATCCTTTAGGTATCTTTCCACAGCCAGGATTCTTGCCTGGAGCTGCTTGATGCCCCAGAC
TGTGAGTTGCAACAGATGCTGTTGCGCCTCAATAGCCCTCAGCAAATTGTTCTGCTGCTGCACTATACCA
GACAATAATTGTCTGGCCTGTACCGTCAGCGTCATTGACGCTGCGCCCATAGTGCTTCCTGCTGCTCCCA
AGAACCCAAGGAACAAAGCTCCTATTCCCACTGCTCTTTTTTCTCTCTGCACCACTCTTCTCTTTGCCTT
GGTGGGTGCTACTCCTAATGGTTCAATTTTTACTACTTTATATTTATATAATTCACTTCTCCAATTGTCC
CTCATATCTCCTCCTCCAGGTCTGAAGATCAGCGGCCGCTTGCTGTGCGGTGGTCTTACTTTTGTTTTGC
TCTTCCTCTATCTTGTCTAAAGCTTCCTTGGTGTCTTTTATCTCTATCCTTTGATGCACACAATAGAGGG
TTGCTACTGTATTATATAATGATCTAAGTTCTTCTGATCCTGTCTGAAGGGATGGTTGTAGCTGTCCCAG
TATTTGTCTACAGCCTTCTGATGTTTCTAACAGGCCAGGATTAACTGCGAATCGTTCTAGCTCCCTGCTT
GCCCATACTATATGTTTTAATTTATATTTTTTCTTTCCCCCTGGCCTTAACCGAATTTTTTCCCATCGCG
ATCTAATTCTCCCCCGCTTAATACTGACGCTCTCGCACCCATCTCTCTCCTTCTAGCCTCCGCTAGTCAA
AATTTTTGGCGTACTCACCAGTCGCCGCCCCTCGCCTCTTGCCGTGCGCGCTTCAGCAAGCCGAGTCCTG
CGTCGAGAGAGCTCCTCTGGTTTCCCTTTCGCTTTCAAGTCCCTGTTCGGGCGCCACTGCTAGAGATTTT
CCACACTGACTAAAAGGGTCTGAGGGATCTCTAGTTACCAGAGTCACACAACAGACGGGCACACACTACT
TGAAGCACTCAAGGCAAGCTTTATTGAGGCTTAAGCAGTGGGTTCCCTAGTTAGCCAGAGAGCTCCCAGG
CTCAGATCTGGTCTAACCAGAGAGACCCAGTACAGGCAAAACGCGCTGCTTATATAGACCTCCCACCGTA
CACGCCTACCGCCCATTTGCGTCAATGGGGCGGAGTTGTTACGACATTTTGGAAAGTCCCGTTGATTTTG
GTGCCAAAACAAACTCCCATTGACGTCAATGGGGTGGAGACTTGGAAATCCCCGTGAGTCAAACCGCTAT
CCACGCCCATTGATGTACTGCCAAAACCGCATCACCATGGTAATAGCGATGACTAATACGTAGATGTACT
GCCAAGTAGGAAAGTCCCATAAGGTCATGTACTGGGCATAATGCCAGGCGGGCCATTTACCGTCATTGAC
GTCAATAGGGGGCGTACTTGGCATATGATACACTTGATGTACTGCCAAGTGGGCAGTTTACCGTAAATAC
TCCACCCATTGACGTCAATGGAAAGTCCCTATTGGCGTTACTATGGGAACATACGTCATTATTGACGTCA
ATGGGCGGGGGTCGTTGGGCGGTCAGCCAGGCGGGCCATTTACCGTAAGTTATGTAACGCGGAACTCCAT
ATATGGGCTATGAACTAATGACCCCGTAATTGATTACTATTAATAACTAGTCAATAATCAATGTCAACGC
GTATATCTGGCCCGTACATCGCGAAGCAGCGCAAAACGCCTAACCCTAAGCAGATTCTTCATGCAATTGT
CGGTCAAGCCTTGCCTTGTTGTAGCTTAAATTTTGCTCGCGCACTACTCAGCGACCTCCAACACACAAGC
AGGGAGCAGATACTGGCTTAACTATGCGGCATCAGAGCAGATTGTACTGAGAGTGCACCATAGGGGATCG
GGAGATCTCCCGATCCGTCGACGTCAGGTGGCACTTTTCGGGGAAATGTGCGCGGAACCCCTATTTGTTT
ATTTTTCTAAATACATTCAAATATGTATCCGCTCATGAGACAATAACCCTGATAAATGCTTCAATAATAT
TGAAAAAGGAAGAGTATGAGTATTCAACATTTCCGTGTCGCCCTTATTCCCTTTTTTGCGGCATTTTGCC
TTCCTGTTTTTGCTCACCCAGAAACGCTGGTGAAAGTAAAAGATGCTGAAGATCAGTTGGGTGCACGAGT
GGGTTACATCGAACTGGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTATTGACGCCGGGCAAGAGCAAC
TCGGTCGCCGCATACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGACACCACGATGCCTGT
AGCAATGGCAACAACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACTGGGGCCAGATGGTAA
GCCCTCCCGTATCGTAGTTATCTACACGACGGGGAGTCAGGCAACTATGGATGAACGAAATAGACAGATC
GCTGAGATAGGTGCCTCACTGATTAAGCATTGGTAACTGTCAGACCAAGTTTACTCATATATACTTTAGA
TTGATTTAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGCTTCCCGAAGGGAGA
AAGGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTC
GTCAGGGGGGCGGAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACCGCCTTTGAGT
GAGCTGATACCGCTCGCCGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAG
CTATGACCATGATTACGCCAAGCTCTAGCTAGAGGTCGACGGTATACAGACATGATAAGATACATTGATG
AGTTTGGACAAACCACAACTAGAATGCAGTGAAAAAAATGCTTTATTTGTGAAATTTGTGATGCTATTGC
TTTATTTGTAACCATTATAAGCTGCAATAAACAAGTTGGGGTGGGCGAAGAACTCCAGCATGAGATCCCC
GCGCTGGAGGATCATCCAGCCGGCGTCCCGGAAAACGATTCCGAAGCCCAACCTTTCATAGAAGGCGGCG
GTGGAATCGAAATCTCGTAGCACGTGTCAGTCCTGCTCCTCGGCCACGAAGTGCACGCAGTTGCCGGCCG
GGTCGCGCAGGGCGAACTCCCGCCCCCACGGCTGCTCGCCGATCTCGGTCATGGCCGGCCCGGAGGCGTC
CCGGAAGTTCGTGGACACGACCTCCGACCACTCGGCGTACAGCTCGTCCAGGCCGCGCACCCACACCCAG
GCCAGGGTGTTGTCCGGCACCACCTGGTCCTGGACCGCGCTGATGAACAGGGTCACGTCGTCCCGGACCA
CACCGGCGAAGTCGTCCTCCACGAAGTCCCGGGAGAACCCGAGCCGGTCGGTCCAGAACTCGACCGCTCC
GGCGACGTCGCGCGCGGTGAGCACCGGAACGGCACTGGTCAACTTGGCCATGGTTTAGTTCCTCACCTTG
TCGTATTATACTATGCCGATATACTATGCCGATGATTAATTGTCAACACGTGCTGATCAGATCCGAAAAT
GGATATACAAGCTCCCGGGAGCTTTTTGCAAAAGCCTAGGCCTCCAAAAAAGCCTCCTCACTACTTCTGG
AATAGCTCAGAGGCAGAGGCGGCCTCGGCCTCTGCATAAATAAAAAAAATTAGTCAGCCATGGGGCGGAG
AATGGGCGGAACTGGGCGGAGTTAGGGGCGGGATGGGCGGAGTTAGGGGCGGGACTATGGTTGCTGACTA
ATTGAGATGCATGCTTTGCATACTTCTGCCTGCTGGGGAGCCTGGGGACTTTCCACACCTGGTTGCTGAC
TAATTGAGATGCATGCTTTGCATACTTCTGCCTGCTGGGGAGCCTGGGGACTTTCCACACCCTAACTGAC
ACACATTCCACAGAATTAATTCGCGTTAAATTTTTGTTAAATCAGCTCATTTTTTAACCAATAGGCCGAA
ATCGGCAAAATCCCTTATAAATCAAAAGAATAGACCGAGATAGGGTTGAGTGTTGTTCCAGTTTGGAACA
AGAGTCCACTATTAAAGAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCACTAAATCGGAACCCT
AAAGGGAGCCCCCGATTTAGAGCTTGACGGGGAAAGCCGGCGAACGTGGCGAGAAAGGAAGGGAAGAAAG
CGAAAGGAGCGGGCGCTAGGGCGCTGGCAAGTGTAGCGGTCACGCTGCGCGTAACCACCACACCCGCCGC
GCTTAATGCGCCGCTACAGGGCGCGTGGGGATACCCCCTAGAGCCCCAGCTGGTTCTTTCCGCCTCAGAA
GCCATAGAGCCCACCGCATCCCCAGCATGCCTGCTATTGTCTTCCCAATCCTCCCCCTTGCTGTCCTGCC
CCACCCCACCCCCCAGAATAGAATGACACCTACTCAGACAATGCGATGCAATTTCCTCATTTTATTAGGA
AAGGACAGTGGGAGTGGCACCTTCCAGGGTCAAGGAAGGCACGGGGGAGGGGCAAACAACAGATGGCTGG
CAACTAGAAGGCACAGTCGAGGCTGATCAGCGGGTTTAAACGGGCCCTGCTAGAGATTTTCCACACTGAC
TAAAAGGGTCTGAGGGATCTCTAGTTACCAGAGTCACACAACAGACGGGCACACACTACTTGAAGCACTC
AAGGCAAGCTTTATTGAGGCTTAAGCAGTGGGTTCCCTAGTTAGCCAGAGAGCTCCCAGGCTCAGATCTG
GTCTAACCAGAGAGACCCAGTACAGTCCGGATGCAGCTCTCGGGCCATGTGATGAAATGCTAGGCGGCTG
TCAAACCTCCACTCTAATACTTCTCTCTCCGGGTCATCCATCCCATGCAGGCTCACAGGGTGTAACAAGC
GGGTGTTCTCTCCTTCATTGGCTTCTTCTACCTTCTCTTGCTCAACTGGTACTAGCTTGTAGCACCATCC
AAAGGTCAGTGGATATCTGATCCCTGGCCCTGGTGTGTAGTTCTGCCAATCAGGGAAGTAGCCTTGTGTG
TGGTAGATCCACAGATCAAGGATATCTTGTCTTCGTTGGGAGTGAATTAGCCCTTCCAGTCCCCCCTTTT
CTTTTAAAAAGTGGCTAAGATCTACAGCTGCCTTGTAAGTCATTGGTCTTAAAGGTACCTGAGGTGTGAC
TGGAAAACCCACCTCCTCCTCCTCTTGTGCTTCTAGCCAGGCACAATCAGCATTGGTAGCTGCTGTATTG
CTACTTGTGATTGCTCCATGTTTTTCTAGGTCTCGATCGAGGTCGACGGTA [symbol] CGATGCGGGGAGGCGGCC
CAAAGGGAGATCCGACTCGTCTGAGGGCGAAGGCGAAGACGCGGAAGAGGCCGCAGAGCCGGCAGCAGGC
CGCGGGAAGGAAGGTCCGCTGGATTGAGGGCCGAAGGGACGTAGCAGAAGGACGTCCCGCGCAGAATGCA
GGTGGCAACACAGGCGAGCAGCCAAGGAAAGGACGATGATTTCCCCGACAACACCACGGAATTGTCAGTG
CCCAACAGCCGAGCCCCTGTCCAGCAGCGGGCAAGGCAGGCGGCGATGAGTTCCGCCGTGGCAATAGGGA
GGGGGAAAGCGAAAGTCCCGGAAAGGAGCTGACAGGTGGTGGCAATGCCCCAACCAGTGGGGGTTGCGTC
AGCAAACACAGTGCACACCACGCCACGTTGCCTGACAACGGGCCACAACTCCTCATAAAGAGACAGCAAC
CAGGATTTATACAAGGAGGAGAAAATGAAAGCCATACGGGAAGCAATAGCATGATACAAAGGCATTAAAG
CAGCGTATCCACATAGCGTAAAAGGAGCAACATAGTTAAGAATNCCAGTCAATCTTTCACAAATTTTGTA
ATCCAGAGGTTGATTATCGATAAGCTTGATATCGAATTGGGAGGGACTAGAGTCGCGGCCGCTTTACTTG
TACAGTTTAACATCTCGAGCCATAAAGATGGTTAATTAACCCACCCAAGATC [symbol] AAACCGGTGAGTTTCAT
GGTTACTTGCCTGAGAAGATTAAAAAAAGTAATGCTACCTTATGAGGGAGAGTCCCAGGGACCAAGATAG
CAACTGTCATAGCAACCGTCACACTGCTTTGGTCAAGGAGAAGACCCTTTGGGGAACTGAAAACAGAACC
TTGAGCACATCTGTTGCTTTCGCTCCCATCCTCCTCCAACAGGGCTGGGTGGAGCACTCCACACCCTTTC
ACCGGTCGTACGGCTCAGCCAGAGTAAAAATCACACCCATGACCTGGCCACTGAGGGCTTGATCAATTCA
CTTTGAATTTGGCATTAAATACCATTAAGGTATATTAACTGATTTTAAAATAAGATATATTCGTGACCAT
GTTTTTAACTTTCAAAAATGTAGCTGCCAGTGTGTGATTTTATTTCAGTTGTACAAAATATCTAAACCTA
TAGCAATGTGATTAATAAAAACTTAAACATATTTTCCAGTACCTTAATTCTGTGATAGGAAAATTTTAAT
CTGAGTATTTTAATTTCATAATCTCTAAAATAGTTTAATGATTTGTCATTGTGTTGCTGTCGTTTACCCC
AGCTGATCTCAAAAGTGATATTTAAGGAGATTATTTTGGTCTGCAACAACTTGATAGGGCTCAGCCTCTC
CCACCCAACGGGTGGAATCCCCCAGAGGGGGATTTCCAAGAGGCCACCTGGCAGTTGCTGAGGGTCAGAA
GTGAAGCTAGGCACTTCCTCTTAGGCAGGTGGCCAAGATTACAGTTGACCCGTACGTGCAGCTGTGCCCA
GCCTGCCCCATCCCCTGCTCATTTGCATGTTCCCAGAGCACAACCTCCTGCCCTGAAGCCTTATTAATAG
GCTGGTCACACTTTGTGCAGGAGTCAGACTCAGTCAGGACACAGCTACGAAGCTTGGTACCGAGCTCGGA
TCTCCACCCCGTAGCCACC [symbol] GGGTGTGCCCACTCAGGTCCTGGGGTTGCTGCTGCTGTGGCTTACAGAT
GCCAGATGTGAGATCGAGGAAATTGTGTTGACTCAGAGCCCCGGAACTCTGTCCCTCAGTCCCGGCGAAA
GGGCAACATTCAGTTGTAGGTCTTCCCATTCAATTCGAAGTAGGCGCGTTGCGTGGTACCAACACAAACC
CGGCCAAGCTCCACGCCTGGTGATCCACGGGGTGTCAAATAGGGCATCTGGGATCTCCGATAGGTTTTCA
GGGTCAGGATCAGGTACGGATTTCACCCTTACCATCACACGGGTTGAACCCGAGGATTTCGCCCTTTATT
ACTGCCAGGTATATGGGGCGAGCAGTTATACGTTTGGTCAGGGGACCAAGCTCGAGCGAAAACGGACCGT
CGCGGCCCCAAGCGTCTTTATCTTCCCACCCTCTGACGAACAGCTCAAATCCGGTACAGCTTCTGTAGTT
TGCTTGCTCAACAACTTTTACCCACGGGAGGCGAAAGTTCAATGGAAAGTGGATAATGCCCTTCAATCCG
GGAACAGCCAAGAAAGTGTGACGGAACAGGACAGCAAAGACTCTACCTATTCACTCAGTAGCACTCTGAC
TTTGTCCAAGGCCGATTATGAAAAACATAAGGTTTACGCGTGCGAGGTTACACACCAAGGTCTGCGAAGC
CCAGTAACAAAATCCTTCAATAGGGGTGAGTGCGAGGGCAGAGGAAGTCTGTTAACATGCGGTGACGTCG
AGGAGAATCCTGGCCCA [symbol] AGTCCAACTGGTACAATCCGGAGCTGAAGTGAAAAAACCAGGGGCATCCGT
GAAAGTCTCATGCCAAGCCTCCGGTTACCGATTTAGTAACTTTGTGATACACTGGGTTCGGCAAGCCCCA
GGACAAAGATTCGAGTGGATGGGCTGGATAAACCCATATAATGGTAACAAAGAATTCTCAGCTAAATTCC
AAGACCGGGTAACATTTACGGCGGATACTTCAGCTAACACTGCATACATGGAACTTCGCAGCTTGAGGTC
CGCGGACACAGCCGTATATTACTGCGCGAGGGTTGGCCCATATAGCTGGGACGACAGTCCGCAGGACAAT
TATTACATGGACGTGTGGGGGAAGGGTACGACCGTAATAGTCAGTAGTGCTAGCACCAAGGGCCCATCGG
TCTTCCCCCTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTGGGCA
CCCAGACCTACATCTGCAACGTGAATCACAAGCCCAGCAACACCAAGGTGGACAAGAAAGTTGAGCCCAA
ATCTTGTGACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGTGGTGGTGG
ACGTGAGCCACGAAGACCCTGAGGTCAAGTTCAACTGGTATGTTGACGGCGTGGAGGTGCATAATGCCAA
GACAAAGCCGCGGGAGGAGCAGTACAACAGCACGTACCGTGTGGTCAGCGTCCTCACCGTCCTGCACCAG
GACTGGCTGAATGGCAAGGAGTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTGCCCCCATCCCGGGATGAGCT
GACCAAGAATCAAGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGCTCCTTCT
TCCTCTACTCAAAACTCACCGTGGACAAGAGCAGGTGGCAGCAGGGGAACGTCTTCTCATGCTCCGTGAT
GCATGAGGCTCTGCACAACCACTACACACAGAAGAGCCTCTCCCTGTCTCCGGGTAAATGAGTGCCACGG
CCGGCAAGCCCCCGCTCCCCAGGCTCTCGGGGTCGCGCGAGGATGCTTGGCACGTACCCCGTGTACATAC
TTCCCAGGCACCCAGCATGGAAATAAAGCACCCAGCGCTTCCCTGGGCCCCTGCGAGACTGTGATGGTTC
TTTCCACGGGTCAGGCCGAGTCTGAGGCCTGAGTGGCATGAGGGAGGCAGAGTGGGTCCCACTGTCCCCA
CACTGGCCCAGGCTGTGGTGGGGAGCTGACCTCAGGACATTGTTGGCCCATCCCGGCCGGGCCCTACATC
CTGGGTCCTGCCACAGAGGGAATCACCCCCAGAGGCCCAAGCCCAGGGGGACACAGCACTGACCACCCCC
TTCCTGTCCAGAG [symbol] TGCAACTGGAGGAGAGCTGTGCGGAGGCGCAGGACGGGGAGCTGGACGGGCTGTGG
ACGACCATCACCATCTTCATCACACTCTTCCTGTTAAGCGTGTGCTACAGTGCCACCGTCACCTTCTTCA
AGgtcggccgcacgttgtccccagctgtccttgacattgtcccccatgctgtcacaaactgtctctgaca
ctgtcccacaggctgtccccacctgtccctgacgctgcgggtgggtgggccttgggggcagagaggtggc
ctcagtgccctgaggggtgggtggggctcgggggcagggctgtggcctcgctcacccctgtgctgtgcct
tgcctacagG [symbol] TGAAGTGGATCTTCTCCTCGGTGGTGGACCTGAAGCAGACCATCATCCCCGACTACAGGA
ACATGATCGGACAGGGGGCCTAGGGCCACCCTCTGCGGGGTGTCCAGGGCGCGCCAATAAAATATCTTTA
TTTTCATTACATCTGTGTGTTGGTTTTTTGTGTGAATCGATAGTACTAACATACGCTCTCCATCAAAACA
AAACGAAACAAAACAAACTAGCAAAATAGGCTGTCCCCAGTGCAAGTGCAGGTGCCAGAACATTTCTCTA
TTTAAATGCTAGAATTAACCAAACTGGATCTCTGCTGTCCCTGTAATAAACCCGAAAATTTTGAATTTTT
GTAATTTGTTTTTGTAATTCTTTAGTTTGTATGTCTGTTGCTATTATGTCTACTATTCTTTCCCCTGCAC
TGTACCCCCCAATCCCCCCTTTTCTTTTAAAATTGTGGATGAATACTGCCATTTGTCTGCAGAATTGGCG
CACGCAGTGCCGATCCGTTCACTAATCGAATGGATCTGTCTCTGTCTCTCTCTCCACCTTCTTCTTCTAT
TCCTTCGGGCCTGTCGGGTCCCCTCGGGGTTGGGAGGTGGGTCTGAAACGATAATGGTGAATATCCCTGC
CTAACTCTATTCACTATAGAAAGTACAGCAAAAACTATTCTTAAACCTACCAAGCCTCCTACTATCATTA
TGAATAATTTTATATACCACAGCCAATTTGTTATGTTAAACCAATTCCACAAACTTGCCCATTTATCTAA
TTCCAATAATTCTTGTTCATTCTTTTCTTGCTGGTTTTGCGATTCTTCAATTAAGGAGTGTATTAAGCT

TABLE 8
Coding sequences for FAM2 anti-PE vector (SEQ ID NO: 42). WPRE, woodchuck
hepatitis virus posttranscriptional regulatory element (#); FEEK, B cell
specific promoter (†); T2A, 2A peptide from Thosea asigna virus (italics);
M1 (*) and M2 ( [symbol] ), transmembrane coding regions; anti-PE Light Chain (Δ),
anti-PE Heavy Chain (V) Intron (lower case),

TGTGTAATTGTTAATTTCTCTGTCCCACTCCATCCAGGTCGTGTGATTCCAAATCTGTTCCAGAGATTTA
TTACTCCAACTAGCATTCCAAGGCACAGCAGTGGTGCAAATGAGTTTTCCAGAGCAACCCCAAATCCCCA
GGAGCTGTTGATCCTTTAGGTATCTTTCCACAGCCAGGATTCTTGCCTGGAGCTGCTTGATGCCCCAGAC
TGTGAGTTGCAACAGATGCTGTTGCGCCTCAATAGCCCTCAGCAAATTGTTCTGCTGCTGCACTATACCA
GACAATAATTGTCTGGCCTGTACCGTCAGCGTCATTGACGCTGCGCCCATAGTGCTTCCTGCTGCTCCCA
AGAACCCAAGGAACAAAGCTCCTATTCCCACTGCTCTTTTTTCTCTCTGCACCACTCTTCTCTTTGCCTT
GGTGGGTGCTACTCCTAATGGTTCAATTTTTACTACTTTATATTTATATAATTCACTTCTCCAATTGTCC
CTCATATCTCCTCCTCCAGGTCTGAAGATCAGCGGCCGCTTGCTGTGCGGTGGTCTTACTTTTGTTTTGC
TCTTCCTCTATCTTGTCTAAAGCTTCCTTGGTGTCTTTTATCTCTATCCTTTGATGCACACAATAGAGGG
TTGCTACTGTATTATATAATGATCTAAGTTCTTCTGATCCTGTCTGAAGGGATGGTTGTAGCTGTCCCAG
TATTTGTCTACAGCCTTCTGATGTTTCTAACAGGCCAGGATTAACTGCGAATCGTTCTAGCTCCCTGCTT
GCCCATACTATATGTTTTAATTTATATTTTTTCTTTCCCCCTGGCCTTAACCGAATTTTTTCCCATCGCG
ATCTAATTCTCCCCCGCTTAATACTGACGCTCTCGCACCCATCTCTCTCCTTCTAGCCTCCGCTAGTCAA
AATTTTTGGCGTACTCACCAGTCGCCGCCCCTCGCCTCTTGCCGTGCGCGCTTCAGCAAGCCGAGTCCTG
CGTCGAGAGAGCTCCTCTGGTTTCCCTTTCGCTTTCAAGTCCCTGTTCGGGCGCCACTGCTAGAGATTTT
CCACACTGACTAAAAGGGTCTGAGGGATCTCTAGTTACCAGAGTCACACAACAGACGGGCACACACTACT
TGAAGCACTCAAGGCAAGCTTTATTGAGGCTTAAGCAGTGGGTTCCCTAGTTAGCCAGAGAGCTCCCAGG
CTCAGATCTGGTCTAACCAGAGAGACCCAGTACAGGCAAAACGCGCTGCTTATATAGACCTCCCACCGTA
CACGCCTACCGCCCATTTGCGTCAATGGGGCGGAGTTGTTACGACATTTTGGAAAGTCCCGTTGATTTTG
GTGCCAAAACAAACTCCCATTGACGTCAATGGGGTGGAGACTTGGAAATCCCCGTGAGTCAAACCGCTAT
CCACGCCCATTGATGTACTGCCAAAACCGCATCACCATGGTAATAGCGATGACTAATACGTAGATGTACT
GCCAAGTAGGAAAGTCCCATAAGGTCATGTACTGGGCATAATGCCAGGCGGGCCATTTACCGTCATTGAC
GTCAATAGGGGGCGTACTTGGCATATGATACACTTGATGTACTGCCAAGTGGGCAGTTTACCGTAAATAC
TCCACCCATTGACGTCAATGGAAAGTCCCTATTGGCGTTACTATGGGAACATACGTCATTATTGACGTCA
ATGGGCGGGGGTCGTTGGGCGGTCAGCCAGGCGGGCCATTTACCGTAAGTTATGTAACGCGGAACTCCAT
ATATGGGCTATGAACTAATGACCCCGTAATTGATTACTATTAATAACTAGTCAATAATCAATGTCAACGC
GTATATCTGGCCCGTACATCGCGAAGCAGCGCAAAACGCCTAACCCTAAGCAGATTCTTCATGCAATTGT
CGGTCAAGCCTTGCCTTGTTGTAGCTTAAATTTTGCTCGCGCACTACTCAGCGACCTCCAACACACAAGC
AGGGAGCAGATACTGGCTTAACTATGCGGCATCAGAGCAGATTGTACTGAGAGTGCACCATAGGGGATCG
GGAGATCTCCCGATCCGTCGACGTCAGGTGGCACTTTTCGGGGAAATGTGCGCGGAACCCCTATTTGTTT
ATTTTTCTAAATACATTCAAATATGTATCCGCTCATGAGACAATAACCCTGATAAATGCTTCAATAATAT
TGAAAAAGGAAGAGTATGAGTATTCAACATTTCCGTGTCGCCCTTATTCCCTTTTTTGCGGCATTTTGCC
TTCCTGTTTTTGCTCACCCAGAAACGCTGGTGAAAGTAAAAGATGCTGAAGATCAGTTGGGTGCACGAGT
GGGTTACATCGAACTGGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTATTGACGCCGGGCAAGAGCAAC
TCGGTCGCCGCATACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGACACCACGATGCCTGT
AGCAATGGCAACAACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACTGGGGCCAGATGGTAA
GCCCTCCCGTATCGTAGTTATCTACACGACGGGGAGTCAGGCAACTATGGATGAACGAAATAGACAGATC
GCTGAGATAGGTGCCTCACTGATTAAGCATTGGTAACTGTCAGACCAAGTTTACTCATATATACTTTAGA
TTGATTTAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGCTTCCCGAAGGGAGA
AAGGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTC
GTCAGGGGGGCGGAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACCGCCTTTGAGT
GAGCTGATACCGCTCGCCGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAG
CTATGACCATGATTACGCCAAGCTCTAGCTAGAGGTCGACGGTATACAGACATGATAAGATACATTGATG
AGTTTGGACAAACCACAACTAGAATGCAGTGAAAAAAATGCTTTATTTGTGAAATTTGTGATGCTATTGC
TTTATTTGTAACCATTATAAGCTGCAATAAACAAGTTGGGGTGGGCGAAGAACTCCAGCATGAGATCCCC
GCGCTGGAGGATCATCCAGCCGGCGTCCCGGAAAACGATTCCGAAGCCCAACCTTTCATAGAAGGCGGCG
GTGGAATCGAAATCTCGTAGCACGTGTCAGTCCTGCTCCTCGGCCACGAAGTGCACGCAGTTGCCGGCCG
GGTCGCGCAGGGCGAACTCCCGCCCCCACGGCTGCTCGCCGATCTCGGTCATGGCCGGCCCGGAGGCGTC
CCGGAAGTTCGTGGACACGACCTCCGACCACTCGGCGTACAGCTCGTCCAGGCCGCGCACCCACACCCAG
GCCAGGGTGTTGTCCGGCACCACCTGGTCCTGGACCGCGCTGATGAACAGGGTCACGTCGTCCCGGACCA
CACCGGCGAAGTCGTCCTCCACGAAGTCCCGGGAGAACCCGAGCCGGTCGGTCCAGAACTCGACCGCTCC
GGCGACGTCGCGCGCGGTGAGCACCGGAACGGCACTGGTCAACTTGGCCATGGTTTAGTTCCTCACCTTG
TCGTATTATACTATGCCGATATACTATGCCGATGATTAATTGTCAACACGTGCTGATCAGATCCGAAAAT
GGATATACAAGCTCCCGGGAGCTTTTTGCAAAAGCCTAGGCCTCCAAAAAAGCCTCCTCACTACTTCTGG
AATAGCTCAGAGGCAGAGGCGGCCTCGGCCTCTGCATAAATAAAAAAAATTAGTCAGCCATGGGGCGGAG
AATGGGCGGAACTGGGCGGAGTTAGGGGCGGGATGGGCGGAGTTAGGGGCGGGACTATGGTTGCTGACTA
ATTGAGATGCATGCTTTGCATACTTCTGCCTGCTGGGGAGCCTGGGGACTTTCCACACCTGGTTGCTGAC
TAATTGAGATGCATGCTTTGCATACTTCTGCCTGCTGGGGAGCCTGGGGACTTTCCACACCCTAACTGAC
ACACATTCCACAGAATTAATTCGCGTTAAATTTTTGTTAAATCAGCTCATTTTTTAACCAATAGGCCGAA
ATCGGCAAAATCCCTTATAAATCAAAAGAATAGACCGAGATAGGGTTGAGTGTTGTTCCAGTTTGGAACA
AGAGTCCACTATTAAAGAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCACTAAATCGGAACCCT
AAAGGGAGCCCCCGATTTAGAGCTTGACGGGGAAAGCCGGCGAACGTGGCGAGAAAGGAAGGGAAGAAAG
CGAAAGGAGCGGGCGCTAGGGCGCTGGCAAGTGTAGCGGTCACGCTGCGCGTAACCACCACACCCGCCGC
GCTTAATGCGCCGCTACAGGGCGCGTGGGGATACCCCCTAGAGCCCCAGCTGGTTCTTTCCGCCTCAGAA
GCCATAGAGCCCACCGCATCCCCAGCATGCCTGCTATTGTCTTCCCAATCCTCCCCCTTGCTGTCCTGCC
CCACCCCACCCCCCAGAATAGAATGACACCTACTCAGACAATGCGATGCAATTTCCTCATTTTATTAGGA
AAGGACAGTGGGAGTGGCACCTTCCAGGGTCAAGGAAGGCACGGGGGAGGGGCAAACAACAGATGGCTGG
CAACTAGAAGGCACAGTCGAGGCTGATCAGCGGGTTTAAACGGGCCCTGCTAGAGATTTTCCACACTGAC
TAAAAGGGTCTGAGGGATCTCTAGTTACCAGAGTCACACAACAGACGGGCACACACTACTTGAAGCACTC
AAGGCAAGCTTTATTGAGGCTTAAGCAGTGGGTTCCCTAGTTAGCCAGAGAGCTCCCAGGCTCAGATCTG
GTCTAACCAGAGAGACCCAGTACAGTCCGGATGCAGCTCTCGGGCCATGTGATGAAATGCTAGGCGGCTG
TCAAACCTCCACTCTAATACTTCTCTCTCCGGGTCATCCATCCCATGCAGGCTCACAGGGTGTAACAAGC
GGGTGTTCTCTCCTTCATTGGCTTCTTCTACCTTCTCTTGCTCAACTGGTACTAGCTTGTAGCACCATCC
AAAGGTCAGTGGATATCTGATCCCTGGCCCTGGTGTGTAGTTCTGCCAATCAGGGAAGTAGCCTTGTGTG
TGGTAGATCCACAGATCAAGGATATCTTGTCTTCGTTGGGAGTGAATTAGCCCTTCCAGTCCCCCCTTTT
CTTTTAAAAAGTGGCTAAGATCTACAGCTGCCTTGTAAGTCATTGGTCTTAAAGGTACCTGAGGTGTGAC
TGGAAAACCCACCTCCTCCTCCTCTTGTGCTTCTAGCCAGGCACAATCAGCATTGGTAGCTGCTGTATTG
CTACTTGTGATTGCTCCATGTTTTTCTAGGTCTCGATCGAGG [symbol] CGACGGTATCGATGCGGGGAGGCGGCC
CAAAGGGAGATCCGACTCGTCTGAGGGCGAAGGCGAAGACGCGGAAGAGGCCGCAGAGCCGGCAGCAGGC
CGCGGGAAGGAAGGTCCGCTGGATTGAGGGCCGAAGGGACGTAGCAGAAGGACGTCCCGCGCAGAATGCA
GGTGGCAACACAGGCGAGCAGCCAAGGAAAGGACGATGATTTCCCCGACAACACCACGGAATTGTCAGTG
CCCAACAGCCGAGCCCCTGTCCAGCAGCGGGCAAGGCAGGCGGCGATGAGTTCCGCCGTGGCAATAGGGA
GGGGGAAAGCGAAAGTCCCGGAAAGGAGCTGACAGGTGGTGGCAATGCCCCAACCAGTGGGGGTTGCGTC
AGCAAACACAGTGCACACCACGCCACGTTGCCTGACAACGGGCCACAACTCCTCATAAAGAGACAGCAAC
CAGGATTTATACAAGGAGGAGAAAATGAAAGCCATACGGGAAGCAATAGCATGATACAAAGGCATTAAAG
CAGCGTATCCACATAGCGTAAAAGGAGCAACATAGTTAAGAATNCCAGTCAATCTTTCACAAATTTTGTA
ATCCAGAGGTTGATTATCGATAAGCTTGATATCGAATTGGGAGGGACTAGAGTCGCGGCCGCTTTACTTG
TACAGTTTAACATCTCGAGCCATAAAGATGGTTAATTAACCCACCCAAGATC [symbol] AAACCGGTGAGTTTCAT
GGTTACTTGCCTGAGAAGATTAAAAAAAGTAATGCTACCTTATGAGGGAGAGTCCCAGGGACCAAGATAG
CAACTGTCATAGCAACCGTCACACTGCTTTGGTCAAGGAGAAGACCCTTTGGGGAACTGAAAACAGAACC
TTGAGCACATCTGTTGCTTTCGCTCCCATCCTCCTCCAACAGGGCTGGGTGGAGCACTCCACACCCTTTC
ACCGGTCGTACGGCTCAGCCAGAGTAAAAATCACACCCATGACCTGGCCACTGAGGGCTTGATCAATTCA
CTTTGAATTTGGCATTAAATACCATTAAGGTATATTAACTGATTTTAAAATAAGATATATTCGTGACCAT
GTTTTTAACTTTCAAAAATGTAGCTGCCAGTGTGTGATTTTATTTCAGTTGTACAAAATATCTAAACCTA
TAGCAATGTGATTAATAAAAACTTAAACATATTTTCCAGTACCTTAATTCTGTGATAGGAAAATTTTAAT
CTGAGTATTTTAATTTCATAATCTCTAAAATAGTTTAATGATTTGTCATTGTGTTGCTGTCGTTTACCCC
AGCTGATCTCAAAAGTGATATTTAAGGAGATTATTTTGGTCTGCAACAACTTGATAGGGCTCAGCCTCTC
CCACCCAACGGGTGGAATCCCCCAGAGGGGGATTTCCAAGAGGCCACCTGGCAGTTGCTGAGGGTCAGAA
GTGAAGCTAGGCACTTCCTCTTAGGCAGGTGGCCAAGATTACAGTTGACCCGTACGTGCAGCTGTGCCCA
GCCTGCCCCATCCCCTGCTCATTTGCATGTTCCCAGAGCACAACCTCCTGCCCTGAAGCCTTATTAATAG
GCTGGTCACACTTTGTGCAGGAGTCAGACTCAGTCAGGACACAGCTACGAAGCTTGGTACCGAGCTCGGA
TCTCCACCCCGTAGCCACC [symbol] GGGTGTGCCCACTCAGGTCCTGGGGTTGCTGCTGCTGTGGCTTACAGAT
GCCAGATGTGAGATCGAGGACATTGTGTTGACCCAGACACCGGCTATTATGTCTGCCTCACTCGGCGAAA
GAGTAACGATGACTTGCACCGCGAGTTCTAGCGTCAGCTCTAGCTACCTTCATTGGTTTCAGCAAAAACC
CGGGTCAAGTCCAAAGCTCTGGATCTACTCTACATCAAATCTGGCATCCGGTGTTCCTGGTAGGTTCTCC
GGCTCCGGCAGCGAAACTTCTTATTCCCTGATTATTGGCTCCCTCGAGACAGAAGATGCCGCAACCTACT
ACTGTCACCAATATCACCGGAGCCCGCCCACATTTGGAGGAGGAACTAAATTGGAAATCAAGACAGTTGC
AGCGCCTAGCGTATTTATTTTCCCACCCTCAGATGAGCAACTGAAATCCGGGACCGCATCTGTTGTCTGT
CTGCTGAACAACTTCTATCCCCGAGAGGCCAAAGTTCAGTGGAAAGTGGACAATGCGCTTCAATCTGGTA
ACTCTCAAGAATCCGTCACTGAGCAGGATTCTAAGGATTCTACATATAGTCTTTCATCAACCCTTACACT
TAGTAAGGCGGACTATGAAAAACACAAAGTGTACGCATGTGAGGTAACTCACCAGGGGCTGCGCTCCCCG
GTTACCAAAAGTTTTAATCGAGGGGAGTGCGAGGGCAGAGGAAGTCTGTTAACATGCGGTGACGTCGAGG
V
AGAATCCTGGCCCAGAAGTACAGCTGGAACAGAGTGGTGCAGAGTTGGCAAGACCAGGGGCCAGCGTGAA
ACTCTCTTGCAAGGCGAGTGGATATACCTTTACAAGTTACGGTATTTCTTGGGTAAAACAGAGGACGGGG
CAGGGTCTGGAATGGATAGGTGAAATTTACCCTAAATCCGGCAATACATACTATAATGAAAAATTCAAAG
GACGAGCGACACTTACCGCTGATAAGTCTAGTTCCACTGCGTATATGGAACTCCGAAGCCTTACCAGCGA
GGACTCCACAGTGTATTTTTGCGCGCGACAGGGGTACTATGCAAATTCACAATTCACGTACTGGGGTCAA
GGGACACTTGTAACAGTAAGCGCAGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAAAGTTGAGCCCAAATCTTGTGACAAAACTCACACATG
CCCACCGTGCCCAGCACCTGAACTCCTGGGGGGACCGTCAGTCTTCCTCTTCCCCCCAAAACCCAAGGAC
ACCCTCATGATCTCCCGGACCCCTGAGGTCACATGCGTGGTGGTGGACGTGAGCCACGAAGACCCTGAGG
TCAAGTTCAACTGGTATGTTGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGAATCAAGTCAGCCTGAC
CTGCCTGGTCAAAGGCTTCTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTACTCAAAACTCACCGTGG
ACAAGAGCAGGTGGCAGCAGGGGAACGTCTTCTCATGCTCCGTGATGCATGAGGCTCTGCACAACCACTA
CACACAGAAGAGCCTCTCCCTGTCTCCGGGTAAATGAGTGCCACGGCCGGCAAGCCCCCGCTCCCCAGGC
TCTCGGGGTCGCGCGAGGATGCTTGGCACGTACCCCGTGTACATACTTCCCAGGCACCCAGCATGGAAAT
AAAGCACCCAGCGCTTCCCTGGGCCCCTGCGAGACTGTGATGGTTCTTTCCACGGGTCAGGCCGAGTCTG
AGGCCTGAGTGGCATGAGGGAGGCAGAGTGGGTCCCACTGTCCCCACACTGGCCCAGGCTGTGGTGGGGA
GCTGACCTCAGGACATTGTTGGCCCATCCCGGCCGGGCCCTACATCCTGGGTCCTGCCACAGAGGGAATC
ACCCCCAGAGGCCCAAGCCCAGGGGGACACAGCACTGACCACCCCCTTCCTGTCCAGA [symbol] TGCAACTGGA
GGAGAGCTGTGCGGAGGCGCAGGACGGGGAGCTGGACGGGCTGTGGACGACCATCACCATCTTCATCACA
CTCTTCCTGTTAAGCGTGTGCTACAGTGCCACCGTCACCTTCTTCAAGgtcggccgcacgttgtccccag
ctgtccttgacattgtcccccatgctgtcacaaactgtctctgacactgtcccacaggctgtccccacct
gtccctgacgctgcgggtgggtgggccttgggggcagagaggtggcctcagtgccctgaggggtgggtgg
ggctcgggggcagggctgtggcctcgctcacccctgtgctgtgccttgcctacag [symbol] TGAAGTGGATCTTC
TCCTCGGTGGTGGACCTGAAGCAGACCATCATCCCCGACTACAGGAACATGATCGGACAGGGGGCCTAGG
GCCACCCTCTGCGGGGTGTCCAGGGCGCGCCAATAAAATATCTTTATTTTCATTACATCTGTGTGTTGGT
TTTTTGTGTGAATCGATAGTACTAACATACGCTCTCCATCAAAACAAAACGAAACAAAACAAACTAGCAA
AATAGGCTGTCCCCAGTGCAAGTGCAGGTGCCAGAACATTTCTCTATTTAAATGCTAGAATTAACCAAAC
TGGATCTCTGCTGTCCCTGTAATAAACCCGAAAATTTTGAATTTTTGTAATTTGTTTTTGTAATTCTTTA
GTTTGTATGTCTGTTGCTATTATGTCTACTATTCTTTCCCCTGCACTGTACCCCCCAATCCCCCCTTTTC
TTTTAAAATTGTGGATGAATACTGCCATTTGTCTGCAGAATTGGCGCACGCAGTGCCGATCCGTTCACTA
ATCGAATGGATCTGTCTCTGTCTCTCTCTCCACCTTCTTCTTCTATTCCTTCGGGCCTGTCGGGTCCCCT
CGGGGTTGGGAGGTGGGTCTGAAACGATAATGGTGAATATCCCTGCCTAACTCTATTCACTATAGAAAGT
ACAGCAAAAACTATTCTTAAACCTACCAAGCCTCCTACTATCATTATGAATAATTTTATATACCACAGCC
AATTTGTTATGTTAAACCAATTCCACAAACTTGCCCATTTATCTAATTCCAATAATTCTTGTTCATTCTT
TTCTTGCTGGTTTTGCGATTCTTCAATTAAGGAGTGTATTAAGCT

1. A genome-edited primary B cell, wherein the primary B cell comprises a non-clonal cell expressing at least one of CD19, IgM, IgD, CD27⁺, CD21⁺, and CXCR5⁺.

2.-4. (canceled)

5. The genome-edited primary B cell of claim 1, wherein the primary B cell comprises a proliferating cell.

6. (canceled)

7. The genome-edited primary B cell of claim 1, wherein an endogenous gene is deleted.

8. The genome-edited primary B cell of claim 1, wherein an endogenous gene comprises a point mutation.

9. The genome-edited primary B cell of claim 1, the genome-edited primary B cell comprising an exogenous gene.

10. The genome-edited primary B cell of claim 1, wherein the genome-edited primary B cell comprises an endogenous gene comprising a point mutation or an exogenous gene, and wherein at least one of the endogenous gene and the exogenous gene comprises a nucleic acid encoding at least a portion of a B cell receptor (BCR).

11. The genome-edited primary B cell of claim 1, wherein the primary B cell exhibits decreased expression of an endogenous B cell receptor (BCR) relative to a non-genome edited primary B cell.

12. The genome-edited primary B cell of claim 1, wherein the primary B cell comprises a modification that alters expression or activity of CD19.

13. The genome-edited primary B cell of claim 1, wherein the primary B cell comprises a therapeutic cassette comprising a nucleic acid encoding a B cell receptor (BCR) and a nucleic acid encoding a gene to be overexpressed.

14. A method comprising administering to a subject a composition comprising the genome-edited primary B cell of claim 1.

15. The method of claim 14, wherein the method comprises treating or preventing a disease in the subject, and the disease comprises an enzymopathy, a cancer, a precancerous condition, an infection with a pathogen, or a viral infection.

16. A therapeutic cassette comprising a nucleic acid encoding a B cell receptor (BCR) and a nucleic acid encoding a gene to be overexpressed; wherein the nucleic acid encoding the BCR and the nucleic acid encoding the gene to be overexpressed are transcriptionally linked, translationally linked, or both; and wherein the therapeutic cassette comprises an endogenous promoter that drives transcription of the nucleic acid encoding the BCR and the nucleic acid encoding the gene to be overexpressed.

17. The therapeutic cassette of claim 16, wherein the gene to be overexpressed comprises a nucleic acid encoding an enzyme, and wherein the enzyme comprises an enzyme lacking in a subject having an enzymopathy.

18.-20. (canceled)

21. A vector comprising the therapeutic cassette of claim 16.

22. (canceled)

23. The vector of claim 21, wherein the vector comprises at least one of a BaEV-psuedotype lentiviral vector, a VSVg-psuedotype lentiviral vector, a FAM1 lentiviral vector, and a FAM2 lentiviral vector.

24. A primary B cell comprising the therapeutic cassette of claim 16.

25. (canceled)

26. A method comprising
administering the primary B cell of claim 24 to a subject; and
administering an antigen to the subject, wherein the BCR of the therapeutic cassette is specific to the antigen.

27. (canceled)

28. A method comprising editing a genome of a primary B cell, wherein the primary B cell comprises a cell expressing at least one of CD19, IgM, IgD, CD27⁺, CD21⁺, and CXCR5⁺, the method comprising introducing an exogenous protein or an exogenous nucleic acid into the primary B cell.

29. (canceled)

30. The method of claim 28, the method comprising electroporation of the cell to introduce the exogenous protein or the exogenous nucleic acid into the primary B cell.

31. (canceled)

32. The method of claim 30, wherein the method further comprises subjecting the primary B cell to at least one of an activation, a stimulation, and a proliferation step.

33. (canceled)


SEQUENCE LISTING (42 sequences)

SEQ ID NO: 1 - DNA, 21 bp, artificial
  guide RNA
gcggcggtgatggacgggtcc

SEQ ID NO: 2 - DNA, 20 bp, artificial
  guide RNA
gcggcggtgatggacgggtc

SEQ ID NO: 3 - DNA, 21 bp, artificial
  guide RNA
gtctcgccgggtccgcgcggg

SEQ ID NO: 4 - DNA, 20 bp, artificial
  guide RNA
gcctctcgccgggtccgcgc

SEQ ID NO: 5 - DNA, 20 bp, artificial
  guide RNA
gcggacccggcgagaggcgg

SEQ ID NO: 6 - DNA, 21 bp, artificial
  guide RNA
gtcccgccgccgcctctcgcc

SEQ ID NO: 7 - DNA, 21 bp, artificial
  guide RNA
gtccttgcattggtgcgcctc

SEQ ID NO: 8 - DNA, 20 bp, artificial
  guide RNA
gcctgaggcgcaccaatgca

SEQ ID NO: 9 - DNA, 20 bp, artificial
  guide RNA
gcagagttctgtccttgcat

SEQ ID NO: 10 - DNA, 20 bp, artificial
  guide RNA
gcattggtgcgcctcaggcc

SEQ ID NO: 11 - DNA, 21 bp, artificial
  guide RNA
gctaggcccaggagggcggtg

SEQ ID NO: 12 - DNA, 21 bp, artificial
  guide RNA
gtcaggccaggctgcactgct

SEQ ID NO: 13 - DNA, 21 bp, artificial
  guide RNA
gcttctagcgctcggcaccgg

SEQ ID NO: 14 - DNA, 21 bp, artificial
  guide RNA
gcagcgcgggcttctagcgct

SEQ ID NO: 15 - DNA, 20 bp, artificial
  guide RNA
gggcttctagcgctcggcac

SEQ ID NO: 16 - DNA, 21 bp, artificial
  guide RNA
gttctagcgctcggcaccggc

SEQ ID NO: 17 - DNA, 21 bp, artificial
  guide RNA
gaaatgaaggcaggacgcgcc

SEQ ID NO: 18 - DNA, 21 bp, artificial
  guide RNA
gtcatttatccagcagctttt

SEQ ID NO: 19 - DNA, 21 bp, artificial
  guide RNA
gaagcggggactcccgagacc

SEQ ID NO: 20 - DNA, 21 bp, artificial
  guide RNA
gttcaacgtctctcaacagat

SEQ ID NO: 21 - DNA, 20 bp, artificial
  guide RNA
ggggcctcatgtggattccc

SEQ ID NO: 22 - DNA, 20 bp, artificial
  guide RNA
gctgtgctgcagtgcctcaa

SEQ ID NO: 23 - DNA, 20 bp, artificial
  guide RNA
gcttctacctgtgccagccg

SEQ ID NO: 24 - DNA, 21 bp, artificial
  guide RNA
gtctcagaggggggccccggc

SEQ ID NO: 25 - DNA, 20 bp, artificial
  primer
taccctctctgagcctccat

SEQ ID NO: 26 - DNA, 20 bp, artificial
  primer
cctctctccagctccattgt

SEQ ID NO: 27 - DNA, 20 bp, artificial
  guide RNA
ggtggataacgccctccaat

SEQ ID NO: 28 - DNA, 20 bp, artificial
  guide RNA
tcaactgctcatcagatggc

SEQ ID NO: 29 - DNA, 20 bp, artificial
  guide RNA
atccaccttccactgtactt

SEQ ID NO: 30 - DNA, 20 bp, artificial
  guide RNA
attcagcaggcacacaacag

SEQ ID NO: 31 - DNA, 20 bp, artificial
  guide RNA
cctgctctgtgacactctcc

SEQ ID NO: 32 - DNA, 20 bp, artificial
  guide RNA
tctcctgggagttacccgat

SEQ ID NO: 33 - DNA, 20 bp, artificial
  primer
tgcatgttctttgtgggcta

SEQ ID NO: 34 - DNA, 20 bp, artificial
  primer
cacggcgactactgcactta

SEQ ID NO: 35 - DNA, 20 bp, artificial
  primer
gggaggattgggaagacaat

SEQ ID NO: 36 - DNA, 21 bp, artificial
  primer
tgtcttttctccccatagcaa

SEQ ID NO: 37 - DNA, 8614 bp, artificial
  Anti-PE heavy chain/light chain/coIDUA lentiviral co-expression cassette
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttaacgcgcggtgaccctcgaggtcgacggtatcgataagctcgcttcacgagattccagcaggtcgagggacctaataacttcgtatagcatacattatacgaagttatattaagggttccaagcttaagcggccgcgtggataaccgtattaccgccatgcatatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgctagccaagtttgtacaaaaaagcaggctgccaccatggaagtacagctggaacagagtggtgcagagttggcaagaccaggggccagcgtgaaactctcttgcaaggcgagtggatatacctttacaagttacggtatttcttgggtaaaacagaggacggggcagggtctggaatggataggtgaaatttaccctaaatccggcaatacatactataatgaaaaattcaaaggacgagcgacacttaccgctgataagtctagttccactgcgtatatggaactccgaagccttaccagcgaggactccacagtgtatttttgcgcgcgacaggggtactatgcaaattcacaattcacgtactggggtcaagggacacttgtaacagtaagcgcagccagtactaaggggccgagcgtctttcctctggctcctagctcaaaatccacatccggcggtaccgcagcgctcggatgtcttgttaaagattattttccagaacctgtcacagtcagttggaatagcggcgctttgacttccggggtccatacgtttccggcggtgctgcaatcatccggcctctattccctcagctctgttgttacagtgccgagctcctctctgggcactcagacttatatttgcaacgtaaaccacaagccatctaacacaaaagtggataagaaggtagaacctaagagctgtgacaagacgcatacatgcccgccgtgccctgccccagaactgctcggaggaccatccgtattcctctttccccccaagcccaaagacaccctgatgatttcaagaacgccggaggtgacgtgtgttgttgtagatgtctcccacgaagaccccgaagtaaagtttaattggtatgttgatggcgtggaagtccacaacgccaaaaccaagcctagagaagagcagtacaattcaacatatagagtagtgagtgtattgactgtccttcaccaggactggctcaatggtaaggagtataagtgcaaggtgagcaacaaggcgctccccgctcctatagaaaaaactatcagtaaagcaaaaggtcaaccgagagagcctcaggtctatacactcccccccagccgggatgaactgacgaaaaatcaggtctcacttacgtgtctggttaaaggtttctatccgtctgatattgcggtggaatgggaatcaaatggtcagccagaaaacaattacaaaacgacgcccccagtgctggactccgatggctctttcttcctttattccaaacttacagtagataagtcaagatggcaacaagggaacgtgtttagttgcagcgttatgcatgaagcactccataatcactatacccaaaagagcttgtccctttccccagagcttcaactggaggagtcttgcgcagaggcacaggatggtgagctggatgggttgtggacgacaataaccatctttataacacttttccttctgtctgtctgctacagcgctactgttacctttttcaaagttaagtggatattcagttcagttgtcgatttgaaacagactatcatcccagactatcggaacatgataggccaaggcgccagagccaaaagaagtggttctggcgcgacgaattttagtttgcttaagcaagccggagatgtggaggaaaatcctggaccgatggacattgtgttgacccagacaccggctattatgtctgcctcactcggcgaaagagtaacgatgacttgcaccgcgagttctagcgtcagctctagctaccttcattggtttcagcaaaaacccgggtcaagtccaaagctctggatctactctacatcaaatctggcatccggtgttcctggtaggttctccggctccggcagcgaaacttcttattccctgattattggctccctcgagacagaagatgccgcaacctactactgtcaccaatatcaccggagcccgcccacatttggaggaggaactaaattggaaatcaagacagttgcagcgcctagcgtatttattttcccaccctcagatgagcaactgaaatccgggaccgcatctgttgtctgtctgctgaacaacttctatccccgagaggccaaagttcagtggaaagtggacaatgcgcttcaatctggtaactctcaagaatccgtcactgagcaggattctaaggattctacatatagtctttcatcaacccttacacttagtaaggcggactatgaaaaacacaaagtgtacgcatgtgaggtaactcaccaggggctgcgctccccggttaccaaaagttttaatcgaggggagtgcagagccaaaagaagtggttctggcgcgacgaattttagtttgcttaagcaagccggagatgtggaggaaaatcctggaccgatgaggccgctcagaccgagggctgctcttctcgcccttcttgcatccttgctggccgccccccctgtcgcacccgctgaagccccacacctcgtacacgtggatgctgcgcgcgcgctgtggccattgcgccgattttggaggagcacagggttctgtcctcccttgccccactctcaagcggaccaatacgtcctcagttgggaccagcaacttaacctggcctacgttggggcagtgccacatagaggcataaaacaggttcgaacccactggctgctggagttggtaactacacgcggcagtaccggtcgaggtctctcatacaattttactcatctcgacggataccttgacttgctgagggaaaaccaactgttgcccggtttcgaactgatgggtagcgcatccggccactttactgatttcgaggacaaacaacaagtttttgaatggaaggatttggtaagcagcctggcgagaagatatatcggccgatacgggctcgcgcacgtgtctaaatggaattttgaaacgtggaacgaacctgaccatcacgattttgacaacgtcagcatgacaatgcaggggttccttaactattacgacgcgtgttctgaaggtttgagagcggcttcccctgcactccgcttgggagggcccggggacagcttccatacccccccgcggtcaccgttgtcatggggcctcttgcggcactgtcatgatggcacaaatttttttacgggagaagccggtgtgagactggattacattagtctccaccgaaagggagcccgctcaagcattagtatcctggaacaagagaaagtggttgctcagcagattcggcaattgttcccaaaattcgctgacacgccaatatataacgatgaagctgaccccttggtggggtggagtcttcctcaaccttggcgcgccgatgtaacgtacgctgcaatggtggtaaaggtcatagcccaacatcagaacctgctcctcgccaatacgacaagtgcctttccctacgcgcttttgtccaatgataacgcgtttctgagctaccaccctcacccgtttgctcagcgaacattgaccgctaggttccaagtgaataatacacgaccgccccacgtccaactcttgcgcaaacctgttcttaccgcgatggggctcctcgctcttctcgacgaagagcaactctgggcagaagtatcccaagcgggcactgtgctggactccaaccatactgtaggcgtgctggcctccgcccaccgcccccagggaccagcagacgcgtggagggctgcggttctgatatatgccagtgacgacacaagggcgcacccgaataggtcagtcgctgtgactttgcggctgagaggggtcccaccgggtcctggactggtttatgtaacgagatacctggacaatggtctttgctcccccgatggcgagtggcggaggctggggaggcccgtatttccgactgctgaacagtttcgacgaatgcgagcagctgaagatcccgtagcagcagcaccacgccctcttcctgctggaggtagacttacgttgcgaccggccctccgccttccttcactgctcctcgtacatgtctgcgcacgccccgagaagccaccaggccaggtcactcgactccgagcccttccgctcacccaaggacagctcgtccttgtctggtctgacgagcatgtcggtagcaaatgcctttggacgtacgagatacagtttagccaggacggtaaggcatatactcctgttagtaggaagccatctacttttaacctttttgtattcagtcccgacacgggtgctgtcagcgggtcttaccgggtccgcgccctggattattgggccagacccggacctttctccgatcctgtcccctatctcgaggtgcccgtgccaagggggcctcccagccccggcaatccataatgaacccagctttcttgtacaaagtggttgtacaagtaggaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 38 - DNA, 8638 bp, artificial
  B12 heavy chain/light chain/coIDUA lentiviral co-expression cassette
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttaacgcgcggtgaccctcgaggtcgacggtatcgataagctcgcttcacgagattccagcaggtcgagggacctaataacttcgtatagcatacattatacgaagttatattaagggttccaagcttaagcggccgcgtggataaccgtattaccgccatgcatatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgctagccaagtttgtacaaaaaagcaggctgccaccatgcaagtccaactggtacaatccggagctgaagtgaaaaaaccaggggcatccgtgaaagtctcatgccaagcctccggttaccgatttagtaactttgtgatacactgggttcggcaagccccaggacaaagattcgagtggatgggctggataaacccatataatggtaacaaagaattctcagctaaattccaagaccgggtaacatttacggcggatacttcagctaacactgcatacatggaacttcgcagcttgaggtccgcggacacagccgtatattactgcgcgagggttggcccatatagctgggacgacagtccgcaggacaattattacatggacgtgtgggggaagggtacgaccgtaatagtcagtagtgcgtccactaaaggtccctctgtcttcccattggcaccgagttctaagtctacgtctggtggcacagctgcactgggctgtctggtcaaagactatttccccgaaccagttacggttagttggaacagcggagccctgacaagcggagtgcacacgtttccggcggttcttcaatctagtggactctatagcctctcatccgtagtaacggttccatcctcatctcttggaacgcaaacgtatatttgtaacgttaatcataagccttctaatactaaggttgacaaaaaggtagagcctaaatcttgcgataagacgcacacctgcccgccctgtcctgcacccgaacttttggggggtccatcagttttcctgtttcctccaaagccaaaagatactctgatgatctccagaacgccagaggtaacctgcgtcgtagtcgatgtttctcacgaagaccctgaagtgaaatttaactggtacgtagatggtgtggaagtgcacaatgcgaaaactaaaccgcgcgaagaacagtacaactctacttaccgagtcgtgtccgtcctcacggtcctccatcaggactggcttaacggaaaagaatacaaatgtaaagtcagcaacaaagcgctcccagcgcccatagaaaaaacgattagtaaggcgaaaggtcaacctagagagccacaggtatacactctcccaccttcacgggacgaacttacgaaaaaccaggtttcactcacatgtttggtgaaaggattttatcctagcgatattgctgtagaatgggagagtaacgggcaacctgagaacaattataagacaacacctcctgtattggattcagacggtagttttttcttgtatagcaaattgactgttgacaagtcccggtggcaacaggggaatgtcttctcctgctctgtcatgcatgaagcccttcacaaccattacacccagaaaagtttgtccctctctcccgaacttcagctggaagagtcctgcgccgaagcacaagatggagaactcgatggcctgtggacgaccataactatcttcatcactctcttcttgctgagtgtgtgctacagcgccaccgttacttttttcaaggtaaaatggatattctccagcgtggtcgatctcaaacagacaatcattccggattacagaaacatgattgggcagggcgctagagccaaaagaagtggttctggcgcgacgaattttagtttgcttaagcaagccggagatgtggaggaaaatcctggaccgatggaaattgtgttgactcagagccccggaactctgtccctcagtcccggcgaaagggcaacattcagttgtaggtcttcccattcaattcgaagtaggcgcgttgcgtggtaccaacacaaacccggccaagctccacgcctggtgatccacggggtgtcaaatagggcatctgggatctccgataggttttcagggtcaggatcaggtacggatttcacccttaccatcacacgggttgaacccgaggatttcgccctttattactgccaggtatatggggcgagcagttatacgtttggtcaggggaccaagctcgagcgaaaacggaccgtcgcggccccaagcgtctttatcttcccaccctctgacgaacagctcaaatccggtacagcttctgtagtttgcttgctcaacaacttttacccacgggaggcgaaagttcaatggaaagtggataatgcccttcaatccgggaacagccaagaaagtgtgacggaacaggacagcaaagactctacctattcactcagtagcactctgactttgtccaaggccgattatgaaaaacataaggtttacgcgtgcgaggttacacaccaaggtctgcgaagcccagtaacaaaatccttcaataggggtgagtgcagagccaaaagaagtggttctggcgcgacgaattttagtttgcttaagcaagccggagatgtggaggaaaatcctggaccgatgaggccgctcagaccgagggctgctcttctcgcccttcttgcatccttgctggccgccccccctgtcgcacccgctgaagccccacacctcgtacacgtggatgctgcgcgcgcgctgtggccattgcgccgattttggaggagcacagggttctgtcctcccttgccccactctcaagcggaccaatacgtcctcagttgggaccagcaacttaacctggcctacgttggggcagtgccacatagaggcataaaacaggttcgaacccactggctgctggagttggtaactacacgcggcagtaccggtcgaggtctctcatacaattttactcatctcgacggataccttgacttgctgagggaaaaccaactgttgcccggtttcgaactgatgggtagcgcatccggccactttactgatttcgaggacaaacaacaagtttttgaatggaaggatttggtaagcagcctggcgagaagatatatcggccgatacgggctcgcgcacgtgtctaaatggaattttgaaacgtggaacgaacctgaccatcacgattttgacaacgtcagcatgacaatgcaggggttccttaactattacgacgcgtgttctgaaggtttgagagcggcttcccctgcactccgcttgggagggcccggggacagcttccatacccccccgcggtcaccgttgtcatggggcctcttgcggcactgtcatgatggcacaaatttttttacgggagaagccggtgtgagactggattacattagtctccaccgaaagggagcccgctcaagcattagtatcctggaacaagagaaagtggttgctcagcagattcggcaattgttcccaaaattcgctgacacgccaatatataacgatgaagctgaccccttggtggggtggagtcttcctcaaccttggcgcgccgatgtaacgtacgctgcaatggtggtaaaggtcatagcccaacatcagaacctgctcctcgccaatacgacaagtgcctttccctacgcgcttttgtccaatgataacgcgtttctgagctaccaccctcacccgtttgctcagcgaacattgaccgctaggttccaagtgaataatacacgaccgccccacgtccaactcttgcgcaaacctgttcttaccgcgatggggctcctcgctcttctcgacgaagagcaactctgggcagaagtatcccaagcgggcactgtgctggactccaaccatactgtaggcgtgctggcctccgcccaccgcccccagggaccagcagacgcgtggagggctgcggttctgatatatgccagtgacgacacaagggcgcacccgaataggtcagtcgctgtgactttgcggctgagaggggtcccaccgggtcctggactggtttatgtaacgagatacctggacaatggtctttgctcccccgatggcgagtggcggaggctggggaggcccgtatttccgactgctgaacagtttcgacgaatgcgagcagctgaagatcccgtagcagcagcaccacgccctcttcctgctggaggtagacttacgttgcgaccggccctccgccttccttcactgctcctcgtacatgtctgcgcacgccccgagaagccaccaggccaggtcactcgactccgagcccttccgctcacccaaggacagctcgtccttgtctggtctgacgagcatgtcggtagcaaatgcctttggacgtacgagatacagtttagccaggacggtaaggcatatactcctgttagtaggaagccatctacttttaacctttttgtattcagtcccgacacgggtgctgtcagcgggtcttaccgggtccgcgccctggattattgggccagacccggacctttctccgatcctgtcccctatctcgaggtgcccgtgccaagggggcctcccagccccggcaatccataatgaacccagctttcttgtacaaagtggttgtacaagtaggaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 39 - DNA, 11893 bp, artificial
  FAM1 B12 vector
  misc_feature (669)..(669): n is a, c, g, or t
tgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatncctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtacctgaggtgtgactggaaaacccacctcctcctcctcttgtgcttctagccaggcacaatcagcattggtagctgctgtattgctacttgtgattgctccatgtttttctaggtctcgatcgaggtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatgcaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgatatcgaattgggagggactagagtcgcggccgctttacttgtacagtttaacatctcgagccataaagatggttaattaacccacccaagatctaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaaggtagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctacgaagcttggtaccgagctcggatctccaccccgtagccaccatgggtgtgcccactcaggtcctggggttgctgctgctgtggcttacagatgccagatgtgagatcgaggaaattgtgttgactcagagccccggaactctgtccctcagtcccggcgaaagggcaacattcagttgtaggtcttcccattcaattcgaagtaggcgcgttgcgtggtaccaacacaaacccggccaagctccacgcctggtgatccacggggtgtcaaatagggcatctgggatctccgataggttttcagggtcaggatcaggtacggatttcacccttaccatcacacgggttgaacccgaggatttcgccctttattactgccaggtatatggggcgagcagttatacgtttggtcaggggaccaagctcgagcgaaaacggaccgtcgcggccccaagcgtctttatcttcccaccctctgacgaacagctcaaatccggtacagcttctgtagtttgcttgctcaacaacttttacccacgggaggcgaaagttcaatggaaagtggataatgcccttcaatccgggaacagccaagaaagtgtgacggaacaggacagcaaagactctacctattcactcagtagcactctgactttgtccaaggccgattatgaaaaacataaggtttacgcgtgcgaggttacacaccaaggtctgcgaagcccagtaacaaaatccttcaataggggtgagtgcgagggcagaggaagtctgttaacatgcggtgacgtcgaggagaatcctggcccacaagtccaactggtacaatccggagctgaagtgaaaaaaccaggggcatccgtgaaagtctcatgccaagcctccggttaccgatttagtaactttgtgatacactgggttcggcaagccccaggacaaagattcgagtggatgggctggataaacccatataatggtaacaaagaattctcagctaaattccaagaccgggtaacatttacggcggatacttcagctaacactgcatacatggaacttcgcagcttgaggtccgcggacacagccgtatattactgcgcgagggttggcccatatagctgggacgacagtccgcaggacaattattacatggacgtgtgggggaagggtacgaccgtaatagtcagtagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtggtggggagctgacctcaggacattgttggcccatcccggccgggccctacatcctgggtcctgccacagagggaatcacccccagaggcccaagcccagggggacacagcactgaccacccccttcctgtccagagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagggccaccctctgcggggtgtccagggcgcgccaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatttaaatgctagaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagct

SEQ ID NO: 40 - DNA, 11869 bp, artificial
  FAM1 anti PE vector
  misc_feature (669)..(669): n is a, c, g, or t
tgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatncctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtacctgaggtgtgactggaaaacccacctcctcctcctcttgtgcttctagccaggcacaatcagcattggtagctgctgtattgctacttgtgattgctccatgtttttctaggtctcgatcgaggtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatgcaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgatatcgaattgggagggactagagtcgcggccgctttacttgtacagtttaacatctcgagccataaagatggttaattaacccacccaagatctaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaaggtagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctacgaagcttggtaccgagctcggatctccaccccgtagccaccatgggtgtgcccactcaggtcctggggttgctgctgctgtggcttacagatgccagatgtgagatcgaggacattgtgttgacccagacaccggctattatgtctgcctcactcggcgaaagagtaacgatgacttgcaccgcgagttctagcgtcagctctagctaccttcattggtttcagcaaaaacccgggtcaagtccaaagctctggatctactctacatcaaatctggcatccggtgttcctggtaggttctccggctccggcagcgaaacttcttattccctgattattggctccctcgagacagaagatgccgcaacctactactgtcaccaatatcaccggagcccgcccacatttggaggaggaactaaattggaaatcaagacagttgcagcgcctagcgtatttattttcccaccctcagatgagcaactgaaatccgggaccgcatctgttgtctgtctgctgaacaacttctatccccgagaggccaaagttcagtggaaagtggacaatgcgcttcaatctggtaactctcaagaatccgtcactgagcaggattctaaggattctacatatagtctttcatcaacccttacacttagtaaggcggactatgaaaaacacaaagtgtacgcatgtgaggtaactcaccaggggctgcgctccccggttaccaaaagttttaatcgaggggagtgcgagggcagaggaagtctgttaacatgcggtgacgtcgaggagaatcctggcccagaagtacagctggaacagagtggtgcagagttggcaagaccaggggccagcgtgaaactctcttgcaaggcgagtggatatacctttacaagttacggtatttcttgggtaaaacagaggacggggcagggtctggaatggataggtgaaatttaccctaaatccggcaatacatactataatgaaaaattcaaaggacgagcgacacttaccgctgataagtctagttccactgcgtatatggaactccgaagccttaccagcgaggactccacagtgtatttttgcgcgcgacaggggtactatgcaaattcacaattcacgtactggggtcaagggacacttgtaacagtaagcgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtggtggggagctgacctcaggacattgttggcccatcccggccgggccctacatcctgggtcctgccacagagggaatcacccccagaggcccaagcccagggggacacagcactgaccacccccttcctgtccagagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagggccaccctctgcggggtgtccagggcgcgccaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatttaaatgctagaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagct

SEQ ID NO: 41 - DNA, 12109 bp, artificial
  FAM2 B12 vector
  misc_feature (7324)..(7324): n is a, c, g, or t
tgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtacctgaggtgtgactggaaaacccacctcctcctcctcttgtgcttctagccaggcacaatcagcattggtagctgctgtattgctacttgtgattgctccatgtttttctaggtctcgatcgaggtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatgcaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaatnccagtcaatctttcacaaattttgtaatccagaggttgattatcgataagcttgatatcgaattgggagggactagagtcgcggccgctttacttgtacagtttaacatctcgagccataaagatggttaattaacccacccaagatctaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctaggcacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctacgaagcttggtaccgagctcggatctccaccccgtagccaccatgggtgtgcccactcaggtcctggggttgctgctgctgtggcttacagatgccagatgtgagatcgaggaaattgtgttgactcagagccccggaactctgtccctcagtcccggcgaaagggcaacattcagttgtaggtcttcccattcaattcgaagtaggcgcgttgcgtggtaccaacacaaacccggccaagctccacgcctggtgatccacggggtgtcaaatagggcatctgggatctccgataggttttcagggtcaggatcaggtacggatttcacccttaccatcacacgggttgaacccgaggatttcgccctttattactgccaggtatatggggcgagcagttatacgtttggtcaggggaccaagctcgagcgaaaacggaccgtcgcggccccaagcgtctttatcttcccaccctctgacgaacagctcaaatccggtacagcttctgtagtttgcttgctcaacaacttttacccacgggaggcgaaagttcaatggaaagtggataatgcccttcaatccgggaacagccaagaaagtgtgacggaacaggacagcaaagactctacctattcactcagtagcactctgactttgtccaaggccgattatgaaaaacataaggtttacgcgtgcgaggttacacaccaaggtctgcgaagcccagtaacaaaatccttcaataggggtgagtgcgagggcagaggaagtctgttaacatgcggtgacgtcgaggagaatcctggcccacaagtccaactggtacaatccggagctgaagtgaaaaaaccaggggcatccgtgaaagtctcatgccaagcctccggttaccgatttagtaactttgtgatacactgggttcggcaagccccaggacaaagattcgagtggatgggctggataaacccatataatggtaacaaagaattctcagctaaattccaagaccgggtaacatttacggcggatacttcagctaacactgcatacatggaacttcgcagcttgaggtccgcggacacagccgtatattactgcgcgagggttggcccatatagctgggacgacagtccgcaggacaattattacatggacgtgtgggggaagggtacgaccgtaatagtcagtagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtggtggggagctgacctcaggacattgttggcccatcccggccgggccctacatcctgggtcctgccacagagggaatcacccccagaggcccaagcccagggggacacagcactgaccacccccttcctgtccagagctgcaactggaggagagctgtgcggaggcgcaggacggggagctggacgggctgtggacgaccatcaccatcttcatcacactcttcctgttaagcgtgtgctacagtgccaccgtcaccttcttcaaggtcggccgcacgttgtccccagctgtccttgacattgtcccccatgctgtcacaaactgtctctgacactgtcccacaggctgtccccacctgtccctgacgctgcgggtgggtgggccttgggggcagagaggtggcctcagtgccctgaggggtgggtggggctcgggggcagggctgtggcctcgctcacccctgtgctgtgccttgcctacaggtgaagtggatcttctcctcggtggtggacctgaagcagaccatcatccccgactacaggaacatgatcggacagggggcctagggccaccctctgcggggtgtccagggcgcgccaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatttaaatgctagaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagct

SEQ ID NO: 42 - DNA, 12085 bp, artificial
  FAM2 anti PE vector
  misc_feature (7324)..(7324): n is a, c, g, or t
tgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtacctgaggtgtgactggaaaacccacctcctcctcctcttgtgcttctagccaggcacaatcagcattggtagctgctgtattgctacttgtgattgctccatgtttttctaggtctcgatcgaggtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatgcaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaatnccagtcaatctttcacaaattttgtaatccagaggttgattatcgataagcttgatatcgaattgggagggactagagtcgcggccgctttacttgtacagtttaacatctcgagccataaagatggttaattaacccacccaagatctaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctaggcacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctacgaagcttggtaccgagctcggatctccaccccgtagccaccatgggtgtgcccactcaggtcctggggttgctgctgctgtggcttacagatgccagatgtgagatcgaggacattgtgttgacccagacaccggctattatgtctgcctcactcggcgaaagagtaacgatgacttgcaccgcgagttctagcgtcagctctagctaccttcattggtttcagcaaaaacccgggtcaagtccaaagctctggatctactctacatcaaatctggcatccggtgttcctggtaggttctccggctccggcagcgaaacttcttattccctgattattggctccctcgagacagaagatgccgcaacctactactgtcaccaatatcaccggagcccgcccacatttggaggaggaactaaattggaaatcaagacagttgcagcgcctagcgtatttattttcccaccctcagatgagcaactgaaatccgggaccgcatctgttgtctgtctgctgaacaacttctatccccgagaggccaaagttcagtggaaagtggacaatgcgcttcaatctggtaactctcaagaatccgtcactgagcaggattctaaggattctacatatagtctttcatcaacccttacacttagtaaggcggactatgaaaaacacaaagtgtacgcatgtgaggtaactcaccaggggctgcgctccccggttaccaaaagttttaatcgaggggagtgcgagggcagaggaagtctgttaacatgcggtgacgtcgaggagaatcctggcccagaagtacagctggaacagagtggtgcagagttggcaagaccaggggccagcgtgaaactctcttgcaaggcgagtggatatacctttacaagttacggtatttcttgggtaaaacagaggacggggcagggtctggaatggataggtgaaatttaccctaaatccggcaatacatactataatgaaaaattcaaaggacgagcgacacttaccgctgataagtctagttccactgcgtatatggaactccgaagccttaccagcgaggactccacagtgtatttttgcgcgcgacaggggtactatgcaaattcacaattcacgtactggggtcaagggacacttgtaacagtaagcgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtggtggggagctgacctcaggacattgttggcccatcccggccgggccctacatcctgggtcctgccacagagggaatcacccccagaggcccaagcccagggggacacagcactgaccacccccttcctgtccagagctgcaactggaggagagctgtgcggaggcgcaggacggggagctggacgggctgtggacgaccatcaccatcttcatcacactcttcctgttaagcgtgtgctacagtgccaccgtcaccttcttcaaggtcggccgcacgttgtccccagctgtccttgacattgtcccccatgctgtcacaaactgtctctgacactgtcccacaggctgtccccacctgtccctgacgctgcgggtgggtgggccttgggggcagagaggtggcctcagtgccctgaggggtgggtggggctcgggggcagggctgtggcctcgctcacccctgtgctgtgccttgcctacaggtgaagtggatcttctcctcggtggtggacctgaagcagaccatcatccccgactacaggaacatgatcggacagggggcctagggccaccctctgcggggtgtccagggcgcgccaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatttaaatgctagaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagct